(19) United States
(12) Patent Application Publication
(10) Pub. No.: US 2003/0143586 A1
(43) Pub. Date: Jul. 31, 2003

(54) Genetic hypermutability of plants for gene discovery and diagnosis

(76) Inventors: Qimin Chao, Havertown, PA (US); Luigi Grasso, Bala Cynwyd, PA (US); Nicholas C. Nicolaides, Boothwyn, PA (US); Philip M. Sass, Audubon, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/270,839

(22) Filed: Oct. 11, 2002

Related U.S. Application Data
(60) Provisional application No. 60/328,750, filed on Oct. 12, 2001.

(51) Int. Cl. C12Q 1/68

(52) U.S. Cl. 435/6

(57) ABSTRACT

The invention provides methods for identifying polymorphic markers for herbicide resistance in weeds and for generating herbicide susceptible and herbicide resistant weeds by mutagenizing weeds and comparing genetic differences between herbicide resistant and herbicide susceptible weeds. The methods may involve the inhibition of mismatch repair in the weeds through the introduction of dominant negative alleles of mismatch repair genes, through T-DNA insertional mutations, or the use of chemical inhibitors of mismatch repair. The invention also provides polymorphic markers of herbicide resistance and methods and kits to screen for herbicide resistant weeds, such as horseweed, goosegrass and rye grass.

### CROSS-REFERENCE TO RELATED APPLICATIONS

This application claims the benefit of U.S. Provisional Application Serial No. 60/328,750 filed Oct. 12, 2001, the disclosure of which is hereby incorporated by reference in its entirety.

### BACKGROUND OF INVENTION

1. Field of the Invention

This invention relates to the field of genetic isolation and manipulation of weeds and gene targets for the discovery of herbicide tolerant weeds. In particular, it relates to the discovery of genes essential for herbicide tolerance.

2. Background of the Related Art

Herbicide use for crop management is a critical factor for farmers to generate and maintain healthy, productive crops during the growing season in order to achieve maximal economic value from their harvest. Several studies have found an association with the long-term use of a single herbicide and the emergence of resistant weeds to that particular class or type of herbicide, thereby making the risk of decreased crop yields high (DeFelice, M. (1998) “Managing Weed Resistance to Herbicides” Crop Insights, Vol. 8, No. 7). Herbicide resistance in weeds is conceptually no different from the generation of antibiotic resistance that infectious microbes develop over the course of long-term treatment in livestock and man. In plants, a majority of weeds are typically killed by herbicide treatment, however, through the process of natural selection, genetic variants that are naturally resistant to the toxic effects of an herbicide are enriched for, and eventually establish, a significant population of plants that can over grow a field where herbicide treatment is the major source for weed management (Jasieniuk, M. and B. D. Maxwell (1994) “Population genetics and the evolution of herbicide resistance in weeds” Phytoprotection 75(Suppl.):25-35).

Over the past decade, numerous reports have documented the emergence of herbicide-resistant weed populations. The first report of herbicide resistant weeds was documented in 1968, which cited the emergence of strains resistant to triazine-based herbicides. By the year 1991, over 120 weed biotypes have been identified that are resistant to triazine-based herbicides along with the emergence of resistant weeds to more than 15 different herbicide classes throughout the world. As of 1998, more than 195 herbicide resistant weeds have been reported worldwide, highlighting a trend that parallels herbicide usage. While there are clearly many benefits to using herbicide resistant crops, a major disadvantage is the potential emergence of herbicide resistant weeds due to the over-reliance of a single herbicide or closely related class of herbicides.

Many weed specialists throughout the world support the notion that there will likely be an increase in the development of herbicide resistant (HR) weeds or at least a shift in tolerant weed populations as a result of overusing individual herbicides. In the United States, glyphosate resistant (GR) weeds are expected to pose a significant emergence due to the increased use of Roundup Ready® crops like cotton and soybeans, which have seen an explosion of acreage increases across the country. While resistance to non-selective herbicides like Roundup® is thought to occur less rapidly than selective herbicides used in the past, the emergence of GR weeds has already been reported in several types of ryegrass and winter annual weeds, supporting the notion that Roundup® resistant weeds may pose a serious problem for farmers using Roundup Ready® crops in no-till, narrow-spaced crop management systems (Dyer, W. E. (1994) “Resistance to glyphosate” in Herbicide, S. B. Powles and J. A. M. Holtum, eds. Lewis Publishers, Boca Raton, Fla., pp229-241; Hartzler, B. (1998) “Roundup resistant rigid ryegrass” Iowa State University Weed Science Online, (www.weeds.iastate.edu/weednews/rigidryegrass.htm.).

Current methods to identify weed biotype use morphological criteria for classification. Unfortunately, the mere morphology of a weed at the vegetative stage is practically impossible to determine a GR from a glyphosate-susceptible biotype (Wilbur Mountain, Weed Specialist, PA Dept. of Agriculture, personal communication). Genetic analysis in plants through mutagenesis techniques has been hampered by the inability to generate non-biased genome-wide mutations. Thus, there exists a need in the art for methods of determining the genes responsible for herbicide resistance and susceptibility, for effective screening methods to identify herbicide resistant and susceptible plants in the field, and for methods of altering the genotype of herbicide resistant weeds.

### SUMMARY OF THE INVENTION

The invention provides methods for identifying polymorphic markers of herbicide resistance in a plant comprising: (a) isolating genomic DNA from an herbicide susceptible plant and an herbicide resistant plant of the same species; (b) performing genetic analysis on the genomic DNA of the an herbicide susceptible plant and the herbicide resistant plant; and (c) identifying differences between the genomic DNA of the herbicide susceptible plant and the herbicide resistant plant, (d) identifying the differences that correlate with herbicide resistance or herbicide susceptibility by screening samples of herbicide resistant and herbicide susceptible plants; thereby identifying polymorphic markers of herbicide resistance in the plant.

In some embodiments of the method of the invention, the polymorphic markers comprise polynucleotide microsatellite markers where herbicide resistant plants have a distinct haplotype pattern in comparison to herbicide susceptible species.

In some embodiments of the method of the invention, the plant is Conyza canadensis. In other embodiments, the plant is Lolium rigidum. In other embodiments, the plant is a goosegrass species.

In some embodiments of the method of the invention, the herbicide comprises glyphosate. In other embodiments, the herbicide comprises paraquot. In other embodiments, the herbicide comprises sulfonyl urea moities.

The invention also provides methods for generating herbicide susceptible weeds from herbicide resistant weeds comprising: (a) mutagenizing the resistant weeds, thereby creating mutant parental weeds; (b) testing progeny of the mutant parental weeds for susceptibility to the herbicide; and (c) selecting the mutant parental weeds producing herbicide susceptible progeny. In addition, the invention provides methods for generating herbicide resistant weeds from herbicide susceptible weeds comprising: (a) mutagenizing the susceptible weeds, thereby creating mutant parental weeds; (b) testing progeny of the mutant parental weeds for resistance to the herbicide; and (c) selecting the mutant parental weeds producing herbicide resistant progeny.

In some embodiments of the method of the invention, the step of testing comprises analyzing the progeny for resistance to an herbicide selected from the group consisting of aminoglycosides, 5-enolpyruvylshikimate-3-phosphate synthase inhibitors, triazine-based herbicides, beta-lactams, macrolides, lincosamides, sulfonamides, atrazine, alachlor, isoniazids, and metribuzin.

In certain embodiments, the invention provides a method for generating genetically stable glyphosate susceptible weeds derived from glyphosate resistant parental weeds comprising: (a) contacting the glyphosate susceptible weed with an inhibitor of mismatch repair, thereby forming a hypermutable parental weed; (b) testing progeny of the hypermutable parental weed that are glyphosate susceptible; (c) selecting hypermutable parental strains producing glyphosate susceptible progeny; (d) removing the inhibitor of mismatch repair from the hypermutable parental weed, thereby making the hypermutable parental weed genetically stable; and (e) obtaining progeny from genetically stable parental weed.

In some embodiments of the method of the invention, the step of mutagenizing is accomplished by introducing into the herbicide resistant weed a dominant negative allele of a mismatch repair gene. In some embodiments, the dominant negative allele of a mismatch gene is a dominant negative allele of a gene encoding a mismatch repair protein selected from the group consisting of PMS2, PMS1, MLH1, MSH2, MSH6, PMSR2, PMSR3, and PMSL9.

The mismatch repair allele may be derived from any organism, including, but not limited to mouse, human, Arabidopsis, Saccharomyces, and Oryza. In some embodiments, the dominant negative allele is a PMS2 truncation mutant, such as, but not limited to a PMS2-134 mutant.

In other embodiments of the method of the invention, the step of mutagenizing is accomplished by introducing into the herbicide resistant weed a chemical inhibitor of mismatch repair selected from the group consisting of an anthracene, an ATPase inhibitor, a nuclease inhibitor, a polymerase inhibitor and an antisense oligonucleotide that specifically hybridizes to a nucleotide encoding a mismatch repair protein dominant negative allele of a mismatch repair gene. In some embodiments, the chemical inhibitor is an anthracene having the formula:

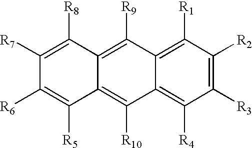

wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein the heteroalkyl, heteroaryl, and substituted heteroaryl contain at least one heteroatom that is oxygen, sulfur, a metal atom, phosphorus, silicon or nitrogen; and wherein the substituents of the substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein the amino groups are optionally substituted with an acyl group, or 1 to 3 aryl or lower alkyl groups. In certain embodiments, R₅ and R₆ are hydrogen. In other embodiments, R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

In specific embodiments, the anthracene derivatives include, but are not limited to 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

In other embodiments of the method of the invention, the step of mutagenizing is accomplished using T-DNA insertional mutagenesis.

The invention also provides a method for identifying a mutant gene conferring herbicide resistance. In some embodiments, the method comprises: (a) comparing the genome of a naturally occurring herbicide resistant plant to the genome of an herbicide susceptible plant; (b) determining genetic differences between the herbicide resistant plant to the herbicide susceptible plant; and (c) sequencing a region of DNA comprising the genetic difference.

In other embodiments, the method comprises: (a) introducing into an herbicide susceptible weed gene fragments from an herbicide resistant weed, thereby creating a transfected herbicide susceptible strain; (b) screening progeny of the transfected herbicide susceptible strain for herbicide resistance; and (c) sequencing the gene fragment to identify an herbicide resistance gene.

In other embodiments, the method comprises: (a) introducing into an herbicide resistant weed gene fragments from an herbicide susceptible weed, thereby creating a transfected herbicide resistant strain; (b) screening progeny of the transfected herbicide resistant strain for herbicide susceptibility; and (c) sequencing the gene fragment to identify an herbicide susceptibility gene.

In still other embodiments, the method comprises: (a) crossing an herbicide resistant weed with an herbicide susceptible weed, thereby creating a crossed strain; (b) screening progeny for herbicide susceptibility; and (c) performing genetic analysis on the crossed strain producing herbicide susceptible progeny to identify an herbicide susceptibility gene.

In still other embodiments, the method comprises: (a) crossing an herbicide resistant weed with an herbicide susceptible weed, thereby creating a crossed strain; (b) screening progeny for herbicide resistance; and (c) performing genetic analysis on the crossed strain producing herbicide resistant progeny to identify an herbicide resistance gene.

In the embodiments of the methods of the invention, the genome of the herbicide resistant plant and the genome of the herbicide susceptible plant are compared by a technique which may include, but is not limited to microarray analysis, genotyping of repetitive sequences using microsatellite markers to identify linked genomic segments that are associated with a particular trait, single nucleotide polymorphic (SNP) analysis, restriction fragment length polymorphism (RFLP) analysis, amplified fragment length polymorphism (AFLP) analysis, simple sequence length polymorphism analysis (SSLPs), randomly amplified polymorphic DNAs (RAPDs), DNA amplification fingerprinting (DAF), sequence characterized amplified regions (SCARs), arbitrary primed polymerase chain reaction (AP-PCR), and single nucleotide polymorphisms (SNPs).

In the methods involving breeding of herbicide susceptible and herbicide resistant weeds, the method may further comprise a step of performing at least one backcross of the progeny with the crossed strain.

The invention also provides methods for identifying a mutant gene conferring herbicide resistance. In some embodiments, the method comprises: (a) mutagenizing an herbicide susceptible weed, thereby creating mutant parental weeds; (b) testing progeny of the mutant parental weeds for resistance to the herbicide; (c) comparing the genome of a naturally occurring herbicide resistant plant to the genome of an herbicide susceptible plant; (d) determining genetic differences between the herbicide resistant plant to the herbicide susceptible plant; and (e) sequencing a region of DNA comprising the genetic difference.

In other embodiments, the method comprises: (a) mutagenizing an herbicide resistant weed, thereby creating mutant parental weeds; (b) testing progeny of the mutant parental weeds for susceptibility to the herbicide; (c) comparing the genome of a naturally occurring herbicide resistant plant to the genome of an herbicide susceptible plant; (d) determining genetic differences between the herbicide resistant plant to the herbicide susceptible plant; and (e) sequencing a region of DNA comprising the genetic difference.

In some embodiments of the method of the invention, the step of mutagenizing is accomplished by introducing into the herbicide resistant weed a dominant negative allele of a mismatch repair gene. In some embodiments, the dominant negative allele of a mismatch gene is a dominant negative allele of a gene encoding a mismatch repair protein selected from the group consisting of PMS2, PMS 1, MLH1, MSH2, MSH6, PMSR2, PMSR3, and PMSL9. The mismatch repair allele may be derived from any organism, including, but not limited to mouse, human, Arabidopsis, Saccharomyces, and Oryza. In some embodiments, the dominant negative allele is a PMS2 truncation mutant, such as, but not limited to a PMS2-134 mutant.

In other embodiments of the method of the invention, the step of mutagenizing is accomplished by introducing into the herbicide resistant weed a chemical inhibitor of mismatch repair selected from the group consisting of an anthracene, an ATPase inhibitor, a nuclease inhibitor, a polymerase inhibitor and an antisense oligonucleotide that specifically hybridizes to a nucleotide encoding a mismatch repair protein dominant negative allele of a mismatch repair gene. In some embodiments, the chemical inhibitor is an anthracene having the formula:

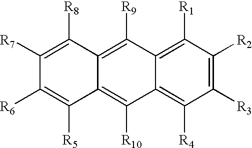

wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein the heteroalkyl, heteroaryl, and substituted heteroaryl contain at least one heteroatom that is oxygen, sulfur, a metal atom, phosphorus, silicon or nitrogen; and wherein the substituents of the substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein the amino groups are optionally substituted with an acyl group, or 1 to 3 aryl or lower alkyl groups. In certain embodiments, R₅ and R₆ are hydrogen. In other embodiments, R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

In specific embodiments, the anthracene derivatives include, but are not limited to 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

In other embodiments of the method of the invention, the step of mutagenizing is accomplished using T-DNA insertional mutagenesis.

The invention also provides polymorphic DNA markers for identifying herbicide resistant and herbicide susceptible weeds. The polymorphic marker comprises a polynucleotide sequence encoding a polypeptide comprising SEQ ID NO: 17. In some embodiments, the polymorphic DNA marker comprises the polynucleotide sequence of SEQ ID NO: 16 or SEQ ID NO: 126. In other embodiments, the polymorphic marker comprises a sequence encoding a polypeptide that is at least 75%, 80%, 85%, 90%, 95%, or 99% identical to SEQ ID NO: 17. In some embodiments, the homolog has the sequence of SEQ ID NO: 60. In other embodiments, the homolog has a sequence of SEQ ID NO: 61. In other embodiments, the homologs have the sequences of SEQ ID NO: 127, or SEQ ID NO: 128. The homologs have nucleic acid sequences that are at least 80% identical to that of SEQ ID NO: 126. Preferably, the nucleic acid sequence is at least 85-90% identical to that of SEQ ID NO: 126. More preferably, the nucleic acid sequence is at least 90-95% identical to that of SEQ ID NO: 126. Even more preferably, the nucleic acid sequence is at least 95-99% identical to that of SEQ ID NO: 126.

The invention also provides a kit for the identification of herbicide resistant and herbicide susceptible weeds comprising, in one or more containers, an oligonucleotide primer comprising the sequence of SEQ ID NO: 18, and a second oligonucleotide primer comprising the sequence of SEQ ID NO: 19. In some embodiments, the kit may further comprise a DNA polymerase, deoxynucleotide triphosphates, genomic DNA from an herbicide susceptible plant, genomic DNA from an herbicide resistant plant, and/or a DNA polymerase buffer.

### BRIEF DESCRIPTION OF DRAWINGS

FIG. 1 shows a genetic analysis of Glycine max (soybean) cultivars using the MOR-1117 marker. PCR analysis of DNA from Am (A1), Wi81 (B1), and CL(C1) cultivars using allele-specific primers demonstrates the ability to distinguish cultivars at the genetic level. PCR analysis of DNA from other plants such as Arabidopsis did not result in a DNA product demonstrating the specificity of this assay (not shown). Arrows indicate products of expected molecular size.

FIG. 2 shows a gel analysis of PCR-amplified fragments (A) shows a fluorescence histogram of HR DNA, (B) is a fluorescence histogram for HS DNA. A 140 bp fragment was identified to be present only in HR but absent in HS horseweed genomic DNA (arrow).

FIG. 3 shows a Southern blot using a cloned polymorphic fragment of Conyza canadensis as a probe for HS and HS weed DNA. Lane 1 and 3 are amplified products from HS, while lane 2 and 4 are those from HR. Lane 5 is the insert from the clone. Lanes 1 and 2 are amplified with dye-labeled 2-TG primer while lanes 3 and 4 are amplified with unlabeled 2-TG.

FIG. 4 shows a typical PCR amplification for the MOR9 marker in HS and HR horseweed.

FIG. 5 shows amplification of MOR9 Homolog 1 (MOR9 H1) from genomic DNA of glyphosate resistant (lane 1) and glyphosate susceptible (lane 2) horseweed. Lane 3 shows an amplification reaction in which no DNA was added.

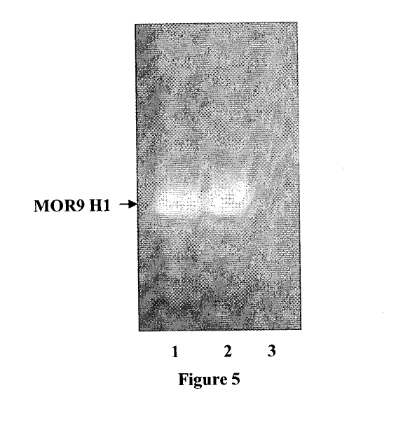

FIG. 6 shows amplification of MOR9 Homolog 2 (MOR9 H2) from genomic DNA of glyphosate resistant (lane 1) and glyphosate susceptible (lane 2) horseweed. Lane 3 shows an amplification reaction in which no DNA was added.

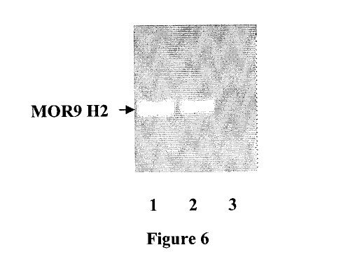

FIG. 7 shows amplification of SSR markers HGA1, HGA2 and HGA3 from different biotypes of horseweed (Lane 1: glyphosate susceptible; Lanes 2-4: glyphosate resistant; and Lane 5: no DNA added). Each marker was amplified with two different pairs of primers: Panel (A) shows three groups of amplifications. The first group shows the results of amplification using NP1-HGA2 and D-HGA2; the second group shows the results of amplification using NP2-HGA2 and D-HGA2; the third group shows the results of amplification using NP1-HGA3 and D-HGA3. Panel (B) shows three groups of amplifications. The first group shows the results of amplification using NP2-HGA3 and D-HGA3; the second group shows the results of amplification using NP1-HGA1 and D-HGA1; the third group shows the results of amplification using NP2-HGA1 and D-HGA1.

### DETAILED DESCRIPTION OF THE INVENTION

The referenced patents, patent applications, and scientific literature, including accession numbers to GenBank database sequences, referred to herein are hereby incorporated by reference in their entirety. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Standard reference works setting forth the general principles of recombinant DNA technology known to those of skill in the art include, but are not limited to Ausubel et al. CURRENT PROTOCOLS IN MOLECULAR BIOLOGY, John Wiley & Sons, New York (1998); Sambrook et al. MOLECULAR CLONING: A LABORATORY MANUAL, 2D ED., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991).

As used herein, “breeding” refers to the art and science of improving a species of plant or animal through controlled genetic manipulation.

As used herein “trait” refers to an observable characteristic of an organism.

As used herein “trait allele” refers to a gene with a defined contribution to an observed characteristic.

As used herein “trait locus” refers to a genetically defined location for a collection of one or more genes (alleles) which contribute to an observed characteristic.

As used herein “weed” refers to undesired vegetation such as that can infiltrate commercial crops and domestic plantings. Non-limiting examples of such undesirable vegetation includes, but is not limited to black mustard (Brassica nigra), curly dock (Rumex crispus), common groundsel (Senecio vulgaris), pineapple weed (Matricaria matricarioides), swamp smartweed (kelp) (Polygonum coccineum), prickly lettuce (Lactuca scariola), lance-leaved groundcherry (Physalis lanceifolia), annual sowthistle (Sonchus oleraceus), London rocket (Sisymbrium irio), common fiddleneck (Amsinckia intermedia), hairy nightshade (Solanum sarrachoides), shepherd's purse (Capsella bursa-pastoris), common knotweed (Polygonum aviculare), green amaranth (Amaranthus hybridus), horseweed (Conyza canadensis), henbit (Lamium amplexicaule), cocklebur (Xanthium strumarium), cheeseweed (Malva parviflora), lambsquarters (Chenopodium album), puncture vine (Tribulus terrestris), common purslane (Portulaca oleracea), prostrate spurge (Euphorbia supina), telegraph plant (Heterotheca grandiflora), carpetweed (Mollugo verticillate), yellow starthistle (Centaurea solstitialis), milk thistle (Silybum marianum), mayweed (Anthemis cotula), burning nettle (Urtica urens), fathen (Atriplex patula), chickweed (Stellaria media), scarlet pimpernel (Anagallis arvensis), redroot pigweed (Amaranthus retroflexus), minners-lettuce (Montia perfoliata), turkey mullein (Eremocarpus setigerus), nettleleaf goosefoot (Chenopodium murale), prostrate pigweed (Amaranthus blitoides), silverleaf nightshade (Solanum elaeagnifolium), hoary cress (Cardaria draba), largeseed dodder (Cuscuta indecora), California burclover (Medicago polymorpha), horse purslane (Trianthema portulacastrum), field bindweed (Convolvulus arvensis), Russian knapweed (Centaurea repens), flax-leaved fleabane (Conyza bonariensis), wild radish (Raphanus sativus), tumble pigweed (Amaranthus albus), stephanomeria (Stephanomeria exigua), wild turnip (Brassica campestris), buffalo goard (Cucurbita foetidissima), common mullein (Verbascum thapsus), dandelion (Taraxacum officinale), Spanish thistle (Xanthium spinosum), chicory (Cichorium intybus), sweet anise (Foeniculum vulgare), annual yellow sweetclover (Melilotus indical), poison hemlock (Conium maculatum), broadleaf filaree (Erodium botrys), whitestem filaree (Erodium moschatum), redstem filaree (Erodium cicutarium), ivyleaf morning-glory (Ipomea hederacea), shortpod mustard (Brassica geniculata), buckhorn plantain (Plantago lacenolata), sticky chickweed (Cerastium viscosum), himalaya blackberry (Rubus procerus), purslane speedwell (Veronica peregrina), mexicantea (Chenopodium ambrosioides), Spanish clover (Lotus purshianus), Australian brassbuttons (Cotula australia), goldenrod (Solidago californica), citron (Citrullus lanatus), hedge mustard (Sisymbrium orientale), black nightshade (Solanum nodiflorum), chinese thornapple (Datura ferox), bristly oxtongue (Picris echioides), bull thistle (Cirsium vulgare), spiny sowthistle (Sonchus asper), tasmanian goosefoot (Chenopodium pumilio), goosefoot (Chenopodium botrys), wright groundcherry (Physalis acutifolia), tomatillo groundcherry (Physalis philadelphica), pretty spurge (Euphorbia peplus), bitter apple (Cucumis myriocarpus), Indian tobacco (Nicotiana bigelovii), common morning-glory (Ipomoea purpurea), waterplantain (Alisma triviale), smartweed (Polygonum lapathifolium), mature sowthistle (Sonchus asper), yellow nutsedge (Cyperus esculentus), purple nutsedge (Cyperus rotundus), lupine (Lupinus formosus), and grasses of the family Gramineae such as annual rye grass (Lolium spp.), blue grass, water grass, barnyard grass, Bermuda grass, fescue, mat grass, Johnson grass, and the like. The methods of the invention may be used for any of the weeds listed above or any subset thereof.

As used herein “crop species” refers to a plant species which is cultivated by man in order to produce a harvestable product. Non-limiting examples of crop species include soybean, corn, sunflower, rapeseed, wheat, barley, oat, rice and sorghum, tomato, potato, cucumber, onion, carrot, common bean, pepper, and lettuce.

As used herein “phenotypic data” refers to a set of trait observations made from one or more individuals.

As used herein “genetic marker” refers to any morphological, biochemical, or nucleic acid-based phenotypic difference which reveals a DNA polymorphism. Examples of genetic markers include, but are not limited to, RFLPs, RAPDs, AFLPs, allozymes and SSRs.

As used herein “genetic marker locus” refers to a genetically defined location for a collection of one or more DNA polymorphisms revealed by a morphological, biochemical or nucleic acid-bred analysis.

As used herein “genetic marker allele” refers to an observed class of DNA polymorphism at a genetic marker locus. For most types of genetic markers (RFLPS, allozymes, SSRs, AFLPs, RADs), alleles are classified based upon DNA fragment size. Individuals with the same observed fragment size at a marker locus have the same genetic marker allele and thus are of the same allelic class.

“Genomic analysis” as used herein, can involve any of a variety of methods used by those skilled in the art for identifying linked genes by genetic mapping, as well as methods to detect gene mutations and/or differential gene expression, including but not limited to differential gene expression using microarrays, cDNA subtraction, differential protein analysis, complementation assays, single nucleotide polymorphism (SNP) analysis or whole genome sequencing to identify altered loci.

“Herbicides” as used herein refers to compounds that kill or retard the growth of plant tissue. Examples of herbicides include, but are not limited to glyphosate, paraquot, sulfonyl urea moities, aminoglycosides, 5-enolpyruvylshikimate-3-phosphate synthase inhibitors, triazine-based herbicides, beta-lactams, macrolides, lincosamides, sulfonamides, Atrazine, Alachlor, isoniazids, and metribuzin.

As used herein “genotyping” refers to the process of determining the genetic composition of individuals using genetic markers.

As used herein “genotype” refers to the allelic composition of an individual at genetic marker loci under study.

As used herein “breeding population” refers to a genetically heterogeneous collection of plants created for the purpose of identifying one or more individuals with desired phenotypic characteristics.

As used herein, “T-DNA” refers to the DNA sequence, a copy of which gets transferred from Agrobacterium to the plant cell.

As used herein, “T-DNA borders” refers to the DNA sequences that flank the T-DNA.

The term “transforming” or “transformation” refers to the process of introducing DNA into a recipient cell. In some embodiments of the invention, transformation refers to introducing DNA into a recipient plant cell and its subsequent integration into the plant cell's chromosomal DNA. The process of transfection can be carried out in a living plant, or it can be carried out in vitro, e.g., using a suspension of one or more isolated cells in culture. In general, transfection will be carried out using a suspension of cells, or a single cell, but other methods can also be applied as long as a sufficient fraction of the treated cells or tissue incorporates the polynucleotide so as to allow transfected cells to be grown and utilized. The protein product of the polynucleotide may be transiently or stably expressed in the cell. Techniques for transfection are well known. Available techniques for introducing polynucleotides include but are not limited to electroporation, Agrobacterium-mediated transformation, T-DNA-mediated transformation, and particle bombardment. Once a cell has been transfected with the mismatch repair gene, the cell can be grown and reproduced in culture. If the transfection is stable, such that the gene is expressed at a consistent level for many cell generations, then a cell line results. In some embodiments, the DNA comes from a large plasmid in the Agrobacterium known as the Ti (Tumor induction) plasmid. The Ti-plasmid comprises several vir (virulence) genes, whose products are directly involved in T-DNA processing and transfer. Located within the natural T-DNA are genes for plant growth regulators and amino acid derivatives, which are for the sole benefit of the Agrobacterium, but are not necessary for the transfer of the T-DNA and its integration into the plant genome. Natural Ti-plasmids are very large. To make it useful for the purpose of plant transformations, two changes may be made to the Ti-plasmids:

1. All the genes within the T-DNA may be removed and replaced with any DNA sequence that one wants to transfer to the plant cell, such as a dominant negative mismatch repair gene.

2. The T-DNA itself is removed from the Ti-plasmid and is placed on a novel plasmid called the “binary vector.”

Together with the Ti-plasmid, this binary vector co-exists and replicates within Agrobacterium. The binary vector is relatively small it is relatively easy to work with. A copy of a short region of DNA (i.e., the T-DNA) in the binary vector is transferred to the plant cell, where it becomes stably integrated into the plant genome, i.e., the plant cell's chromosomal DNA. The construction of binary vectors containing T-DNAs capable of being inserted into a plant genome via Agrobacterium mediated delivery is known to those skilled in the art. In addition to the DNA sequence of interest, a selectable marker gene can be placed within the T-DNA borders in order to allow selection for plants transformed with the DNA sequence of interest. Such selectable marker genes include aph4, for hygromycin resistance, npt2, for kanamycin resistance, bar for Basta resistance, cp4 for glyphosate resistance. Further information of T-DNA transformation of plant cells may be found in U.S. Pat. No. 6,353,155, the disclosure of which is incorporated herein by reference.

The invention provides methods for identifying polymorphic DNA markers in naturally occurring HR weeds to identify haplotypes of biotypes that are resistant to herbicides for the early diagnosis of HR weeds at the vegetative stage as a method for helping farmers adjust and implement proper crop management strategies.

In the method of the invention, polymorphic markers of herbicide resistance in a plant are identified by: (a) isolating genomic DNA from an herbicide susceptible plant and an herbicide resistant plant;(b) performing genetic analysis on said genomic DNA of said an herbicide susceptible plant and said herbicide resistant plant; and (c) identifying differences between the genomic DNA of said herbicide susceptible plant and said herbicide resistant plant, thereby identifying polymorphic markers of herbicide resistance in said plant.

In some embodiments of the invention, field isolates of weeds that are resistant to a selected class of compound or compounds are isolated and their nucleic acid is extracted. The same is done for subtypes of the weeds that are HS. DNA markers are identified and can be analyzed using a variety of methods for identifying altered nucleotide structures including genotyping of repetitive sequences using microsatellite markers to identify linked genomic segments that are associated with a particular trait, single nucleotide polymorphic (SNP) analysis using a variety methods known to those skilled in the art, as well as standard Restriction Fragment Length Polymorphism (RFLP) (Botstein et al. (1980) Am. J. Hum. Genet. 32:314-331) and Amplified Fragment Length Polymorphism (AFLP) methods (Vos et al., (1995) Nucl. Acids Res. 23:4407-4414). It is understood that mutant genes could also be identified by other types of genetic markers such as, for example, Simple Sequence Length Polymorphisms (SSLPs) (Tautz and Renz (1984) “Simple sequences are ubiquitous repetitive components of eukaryotic genomes” Nucl. Acids Res. 25:12(10):4127-38), Randomly Amplified Polymorphic DNAs (RAPDs) (Williams et al. (1990) “Oligonucleotide Primers of Arbitrary Sequence Amplify DNA Polymorphisms which are Useful as Genetic Markers” Nucleic Acids Res. 18:6531-6535), DNA Amplification Fingerprinting (DAF) (Caetano-Anolles et al. (1991) Biotechnol. 9(6):553-557), Sequence Characterized Amplified Regions (SCARs) (Paran and Michelmore (1993) Theor. Appl. Genet. 85:985-993), Arbitrary Primed Polymerase Chain Reaction (AP-PCR) (Welsh and McClelland (1990) Nucleic Acids Res. 18:7213-7218), Amplified Fragment Length Polymorphisms (AFLPs) and Single Nucleotide Polymorphisms (SNPs) (Wang et al. (1998) “Large-Scale Identification, Mapping, and Genotyping of Single-Nucleotide Polymorphisms in the Human Genome” Science 280:1077-1082). In particular, in some embodiments identification of polymorphic markers for glyphosate resistant plants such as Conyza canadensis, and members of the rigid ryegrass and goosegrass families are identified.

In some embodiments, the polymorphic markers comprise polynucleotide microsatellite markers where herbicide resistant plants have a distinct haplotype pattern in comparison to herbicide susceptible species. In one non-limiting embodiment, polymorphic markers are identified by isolating genomic DNA from glyphosate resistant and susceptible field-isolate weeds, identifying polymorphic DNA sequences containing single nucleotide polymorphisms, polynucleotide tracts comprising of mono-, di-, tri- or tetra-repetitive units, identifying flanking sequences and designing primers that are specific for each locus for analysis using methods as known by those skilled in the art.

The invention also provides methods for identifying genes involved in herbicide resistance and susceptibility comprising: (a) isolating genomic DNA from and herbicide susceptible plant and an herbicide resistant plant of the same species; (b) performing genetic analysis on the genomic DNA of the herbicide susceptible plant and an herbicide resistant plant; (c) identify genetic differences between the genomic DNA of the herbicide susceptible plant and an herbicide resistant plant; and (d) sequence the DNA in the regions comprising the genetic differences. Thus, one can identify the genes associated with herbicide susceptibility and resistance in the plants. The genetic analysis may be by any means known in the art and as described herein.

In some embodiments, DNA fragments derived from an herbicide susceptible plant may be isolated and introduced into an herbicide resistant plant. The herbicide resistant plant then contains DNA fragments with altered sequences that are responsible for the herbicide susceptible phenotype. The recombinant plants may then be screened for herbicide susceptibility and the DNA fragments introduced into the plants may be sequenced to identify the gene candidates responsible for the herbicide susceptible phenotype. Conversely, in other embodiments, DNA fragments derived from an herbicide resistant plant may be isolated and introduced into an herbicide susceptible plant. The herbicide susceptible plant then contains DNA fragments with altered sequences that are responsible for the herbicide resistant phenotype. The recombinant plants may then be screened for herbicide resistance and the DNA fragments introduced into the plants may be sequenced to identify the gene candidates responsible for the herbicide resistance phenotype.

In another embodiment of the invention, genetic analysis is coupled with traditional plant breeding and crossing to provide a method for identifying genes involved in susceptibility and resistance to herbicides. For example, the invention provides a method in which an herbicide resistant strain is crossed with an herbicide susceptible strain and the progeny are screened for herbicide resistance. Progeny that are resistant can be subjected to genetic analysis and compared with genetic analysis of the susceptible strain to determine the genetic differences between the strains. The genes may then be sequenced and identified. Optionally, progeny that are found to be herbicide resistant may be back-crossed one or more times with herbicide susceptible strains and the subsequent progeny re-screened for herbicide resistance. The subsequent progeny should have fewer genetic differences, reducing the number of genes to be identified. In another embodiment, progeny that are found to be herbicide susceptible may be back-crossed one or more times with herbicide resistant strains and the subsequent progeny re-screened for herbicide susceptibility. Again, backcrossing more than once should reduce the number of genetic differences between the strains and reduce the number of genes to be identified and sequenced. The methods for genetic analysis may be any known in the art and as described herein.

Methods for the diagnosis of HR weeds are provided that can be used to screen for weed biotypes to detect HR-weeds at any developmental stage. The methods are useful to farmers, for example, to make proper crop management decisions prior to planting. The invention provides methods to identify DNA markers that are linked to genomes of particular resistant weeds. The invention also provides methods to generate a wide array of genomic alterations in an HR weed's genome that can yield maximal number altered target genes that are capable of eliciting susceptibility to a particular herbicide. Using herbicide susceptible (HS) strain developed by the method of the invention, genome analysis identifies mutant gene(s) that are capable of rendering a plant resistant or susceptible to an herbicide for target identification. Methods of genome analysis are known by those skilled in the art of gene mapping and mutation detection.

The invention also provides methods of using field isolates that are naturally resistant to an herbicide or class of herbicide, in gene mapping studies in conjunction with crossing resistant strains to susceptible strains.

In addition, the invention provides methods for generating mutant offspring from herbicide resistant (HR) weeds to create herbicide susceptible (HS) types from strains that are naturally resistant to particular herbicide or class of herbicides are useful for identifying genes responsible for HR as diagnostic markers as well as for herbicide compound screening and development.

In some embodiments of the methods of the invention, mutations are introduced in the plant species to generate genetic diversity. Previously, a bottleneck to generating genetically diverse plants was the inability to generate nonbiased genome-wide mutations. Many mutagenesis methods used chemical and radiation exposure to generate genomic mutations. A limitation of this approach was that these various methods are usually DNA site-specific or are extremely toxic, therefore limiting the mutation spectra and the opportunity to identify a maximal number of genes, when mutated, that are able to confer resistance to an herbicide. Recently, work by Nicolaides, et al. (1998) Mol. Cell. Biol. 18(3):1635-1641; U.S. Pat. No. 6,146,894) has demonstrated the utility of introducing dominant negative mismatch repair (MMR) mutants into cells to confer global DNA hypermutability. These mutations are in the form of point mutations or small insertion-deletions that are distributed equally throughout the genome. The ability to manipulate the MMR process of a target host organism can lead to an increase in the mutability of the target host genome, leading to the generation of innovative cell subtypes with varying phenotypes from the original wild type cells. Variants can be placed under a specified, desired selective process the result of which is the capacity to select for a novel organism that expresses an altered biological molecule(s) and has a new phenotype. The concept of creating and introducing dominant negative allele of a MMR gene in cells has been documented to result in genetically altered cells (Aronshtam A, and M. G. Marinus (1996) “Dominant negative mutator mutations in the mutL gene of Escherichia coli” Nucleic Acids Res. 24:2498-2504; Wu, T. H. and M. G. Marinus (1994) “Dominant negative mutator mutations in the mutS gene of Escherichia coli” J. Bacteriol. 176:5393-400; Brosh R. M. Jr, and S. W. Matson (1995) “Mutations in motif II of Escherichia coli DNA helicase II render the enzyme nonfunctional in both mismatch repair and excision repair with differential effects on the unwinding reaction” J. Bacteriol. 177:5612-5621; Nicolaides, N. C. et al. (1998) “A naturally occurring hPMS2 mutation can confer a dominant negative mutator phenotype” Mol. Cell. Biol. 18:1635-1641). Furthermore, altered MMR activity has been demonstrated when MMR genes from different species including yeast and mammalian cells are over-expressed (Lipkin S. M. et al. (2000) “MLH3: a DNA mismatch repair gene associated with mammalian microsatellite instability” Nat. Genet. 24:27-35). The inhibition of MMR in organisms has been documented to generate hypermutation in whole organisms (WO 01/61012 (US2002128460); de Wind N. et al. (1995) “Inactivation of the mouse MSH2 gene results in mismatch repair deficiency, methylation tolerance, hyperrecombination, and predisposition to cancer” Cell 82:321-300). The ability to create hypermutable organisms by blocking MMR has great commercial value for the generation of HS plants from naturally HR strains and vice-versa for diagnosis, drug screening, and target discovery of genes involved in these phenotypes in addition to the important utility for the early diagnosis of weeds to aid farmers in deciding the most appropriate crop management systems for maximal harvest potential.

The methods of the invention may employ inhibiting mismatch repair in the weeds by introducing a dominant negative mismatch repair gene into the plant. As used herein, “mismatch repair gene” refers to a gene that encodes one of the proteins of the mismatch repair complex. Although not wanting to be bound by any particular theory of mechanism of action, a mismatch repair complex is believed to detect distortions of the DNA helix resulting from non-complementary pairing of nucleotide bases. The non-complementary base on the newer DNA strand is excised, and the excised base is replaced with the appropriate base which is complementary to the older DNA strand. In this way, cells eliminate many mutations that occur as a result of mistakes in DNA replication. Dominant negative alleles cause a mismatch repair defective phenotype even in the presence of a wild-type allele in the same cell. A non-limiting example of a dominant negative allele of a mismatch repair gene is the human gene hPMS2-134, which carries a truncation mutation at codon 134. The mutation causes the product of this gene to abnormally terminate at the position of the 134th amino acid, resulting in a shortened polypeptide containing the N-terminal 133 amino acids. Such a mutation causes an increase in the rate of mutations which accumulate in cells after DNA replication. Thus, expression of a dominant negative allele of a mismatch repair gene results in impairment of mismatch repair activity, even in the presence of the wild-type allele.

The mismatch repair gene may be a dominant negative mismatch repair gene, including, but not limited to a dominant negative form of PMS2, PMS1, PMSR3, PMSR6, MLH1, GTBP, MSH3, MSH2, MSH6-1, MSH7, or MSH1. A non-limiting example includes a dominant negative truncation mutant of PMS2 (e.g., a PMS2-134 gene) (SEQ ID NO: 34).

Examples of mismatch repair genes sequences and proteins are shown by the following: Yeast MLH1 cDNA (SEQ ID NO: 22); Yeast MLH1 protein (SEQ ID NO: 23); Mouse PMS2 cDNA (SEQ ID NO: 24); mouse PMS2 protein (SEQ ID NO: 25); human PMS2 cDNA (SEQ ID NO: 26); human PMS2 protein (SEQ ID NO: 27); human PMS1 cDNA (SEQ ID NO: 28); human PMS1 protein (SEQ ID NO: 29); human MSH2 cDNA (SEQ ID NO: 30); human MSH2 protein (SEQ ID NO: 31); human MLH1 cDNA (SEQ ID NO: 32); human MLH1 protein (SEQ ID NO: 33); human PMS2-134 cDNA (SEQ ID NO: 34); human PMS2-134 protein (SEQ ID NO: 35); human MSH6 cDNA (SEQ ID NO: 36); human MSH6 protein (SEQ ID NO: 37); human PMSR2 cDNA (SEQ ID NO: 38); human PMSR2 protein (SEQ ID NO: 39); human PMSR3 cDNA (SEQ ID NO: 40); human PMSR3 protein (SEQ ID NO: 41); human PMSL9 cDNA (SEQ ID NO: 42); human PMSL9 protein (SEQ ID NO: 43); Arabidopsis thaliana MSH7 cDNA (SEQ ID NO: 44); Arabidopsis thaliana MSH7 protein (SEQ ID NO: 45); Arabidopsis thaliana MSH2 cDNA (SEQ ID NO: 46); Arabidopsis thaliana MSH2 protein (SEQ ID NO: 47); Arabidopsis thaliana MSH3 cDNA (SEQ ID NO: 48); Arabidopsis thaliana MSH3 protein (SEQ ID NO: 49); Arabidopsis thaliana MSH6-1 cDNA (SEQ ID NO: 50); Arabidopsis thaliana MSH6-1 protein (SEQ ID NO: 51); Arabidopsis thaliana PMS2 cDNA (SEQ ID NO: 52); Arabidopsis thaliana PMS2 protein (SEQ ID NO: 53); Arabidopsis thaliana PMS2-134 cDNA (SEQ ID) NO: 54); Arabidopsis thaliana PMS2-134 protein (SEQ ID NO: 55); Oryza sativa MLH1 cDNA (SEQ ID NO: 56); and Oryza sativa MLH1 protein (SEQ ID NO: 67).

The methods of the invention include the use of chemical inhibitors or mismatch repair to induce mutations in the weeds to convert herbicide resistant weeds into herbicide susceptible weeds, or vice versa. The chemical inhibitors of mismatch repair include, but are not limited to an anthracene, an ATPase inhibitor, a nuclease inhibitor, a polymerase inhibitor and an antisense oligonucleotide that specifically hybridizes to a nucleotide encoding a mismatch repair protein.

In some embodiments, the chemical inhibitor is an anthracene having the formula:

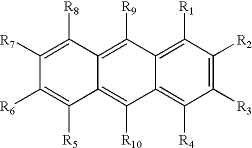

wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein said heteroalkyl, heteroaryl, and substituted heteroaryl contain at least one heteroatom that is oxygen, sulfur, a metal atom, phosphorus, silicon or nitrogen; and wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein said amino groups are optionally substituted with an acyl group, or 1 to 3 aryl or lower alkyl groups. In certain embodiments, R₅ and R₆ are hydrogen. In other embodiments, R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

Non-limiting examples of the anthracenes include 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene. The chemical inhibitors of mismatch repair are more fully described in PCT Publication No. WO 02/054856, which is incorporated by reference in its entirety.

In the method of the invention, polymorphic DNA markers may be identified by genotyping an herbicide resistant plant and an herbicide susceptible plant of the same species. The HR and HS plants may be natural field isolates, modified genetically as described in the methods above, or may have been generated using traditional breeding and selection. The genotyping may be performed using any form of genotyping known in the art, as described above.

In other embodiments of the method of the invention, diversity is generated in the plants by introducing T-DNA into the genome of an herbicide resistant or herbicide susceptible strain. The T-DNA may be used to introduce a dominant negative allele of a mismatch repair gene into the plant.

Methods for transforming dicotyledenous species with Agrobacterium are well established in the art. Recently, it has been shown that monocotyledenous plants may also be transformed using A. tumefaciens transformation methods. It has been shown that the following monocots could be transformed using Agrobacterium transformation: corn (Zea mays L.); wheat (Triticum aestivum L.); rice (Oryza sativa L.); barley (Hordeum vulgare L.); sugar cane (Saccharum spp. L.); (Anthurium scherzerianum Schott ‘Rudolph’ and ‘UH1060’); orchid (Phalaenopsis spp.); and iris (Iris germamica L. ‘Skating Party’) (See Arencibia et al. (1998) Transgenic Res. 7:213-222; Cheng et al. (1997) Plant Physiol. 115:971-980; Hiei et al. (1994) Plant J. 6:271-282; Ishida et al. (1996) Nature Biotechnol. 14:745-750; Tingay et al. (1997) Plant J. 11:1369-1375; Chen and Kuehnle (1996) J. Amer. Soc. Hort. Sci. 121:47-51; Belarmino and Mii (2000) Plant Cell Rpt. 19:435-442; and U.S. Pat. No. 6,459,017).

The invention provides methods for identifying herbicide resistant (HR) forms of weeds to help farmers apply appropriate crop management systems. The ability to identify genome haplotypes in weeds that can determine herbicide resistant (HR) biotypes from herbicide susceptible (HS) biotypes will aid in the rapid analysis of weeds prior to planting and allow for the appropriate design of crop management systems for farmers. For example, with knowledge of the type of herbicide resistance prevalent in the weed population, farmers may choose more appropriate herbicides to control the growth of weeds among their crops.

The invention also provides methods for developing mutant offspring from naturally occurring HR weeds by mutagenesis methods including but not limited to chemical mutagenesis, radiation mutagenesis, or by altering the activity of endogenous mismatch repair (MMR) activity of hosts to generate HS offspring for target discovery. In addition, HS plants are useful for screening chemical libraries to identify novel herbicide agents as well as for the rational design of chemicals for herbicide product development. Mutagens affecting mismatch repair and dominant negative alleles of mismatch repair genes, when applied to plants, are examples of how to mutagenize weeds by increasing the rate of spontaneous mutations through the reduction of MMR-mediated DNA repair activity, thereby rendering plants highly susceptible to genetic alterations due to hypermutability. Hypermutable weeds can be utilized to screen for novel mutations in a gene or a set of genes that produce variant siblings exhibiting new output traits not found in the wild type plants such as HS in plants whereby the parental strain is naturally HR.

The invention also provides a method for screening for HR and HS Conyza canadensis. The method is a PCR-based assay in which plant genomic DNA is amplified with primers that specifically amplify a portion of plant DNA present in HR Conyza canadensis, but not HS Conyza canadensis. The primers used in the assay are PCR Primer 1: 5′-TTG TCG CTG TCC AAC CAT TG-3′ SEQ ID NO: 18); PCR Primer 2: 5′-TTG GCA TGG TCT GTA GCT GG-3′ SEQ ID NO: 19); Control PCR Primer 1: 5′-CCA TCG TAT CAT CAT GTG C-3′ SEQ ID NO: 20); and Control PCR Primer 2: 5′-TGC AAT ATG TTA AAG TAG AGC-3′ SEQ ID NO: 21). PCR Primer 1 (SEQ ID NO: 18) and PCR Primer 2 (SEQ ID NO: 19) specifically amplify a portion of genomic DNA from HR Conyza canadensis, but do not amplify any product from HS Conyza canadensis. Control PCR Primer 1 (SEQ ID NO: 20) and Control PCR Primer 2 (SEQ ID NO: 21) may be used to amplify a product from both HR and HS Conyza canadensis. The conditions of the PCR are not particularly limited, and may be performed following any of the many protocols and variations known in the art. The primers used in the method of the invention may have alterations at the 5′ end to engineer restriction sites, and may have substituted nucleotides throughout the primer provided the oligonucleotide sequence is at least 80% identical to the primers shown for SEQ ID NOs: 18, 19, 20 and 21, and comprise the identical three 3′ nucleotides for each primer.

The method also provides polymorphic markers of Conyza canadensis which may be used to distinguish herbicide resistant or herbicide susceptible plants. The polymorphic markers comprise MOR9 (SEQ ID NO: 16 and SEQ ID NO: 126), as well as homologs MOR9 H1 (SEQ ID NO: 60 and SEQ ID NO: 127), and MOR9 H2 (SEQ ID NO: 61 and SEQ ID NO: 128). The polymorphic markers further comprise the nucleotide sequences of SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, and SEQ ID NO: 111

The invention also provides a kit for screening for HS and HR Conyza canadensis comprising in one or more containers, a set of primers for amplifying a portion of DNA from HR Conyza canadensis. In some embodiments, the kit further comprises other components, such as, but not limited to, a DNA polymerase, dNTPs, control primers, control DNA, DNA polymerase buffer, and instructions for use. In some embodiments, the primers for amplifying a portion of DNA from HR Conyza canadensis, comprise oligonucleotide primers having the sequences of SEQ ID NO: 18 and SEQ ID NO: 19. In some embodiments, the control PCR primers comprise oligonucleotide primers comprising the sequences of SEQ ID NO: 20 and SEQ ID NO: 21.

In general, the oligonucleotide primers that may be used to amplify a polymorphic marker of herbicide resistant plants is at least 15 nucleotides in length and at least 85% identical to a portion of a polymorphic marker selected from the group consisting of SEQ ID NO: 16, SEQ ID NO: 126, SEQ ID NO: 60, SEQ ID NO: 127, SEQ ID NO: 61, SEQ ID NO: 128, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, and SEQ ID NO: 111. The oligonucleotide primers of the invention anneal to a complementary portion of the polymorphic markers under PCR conditions, which are well-known in the art. In some embodiments, the oligonucleotide has a 3′ end that comprises at least 3 identical nucleotides of a portion of at least one of the above polymorphic markers in addition to the other stated characteristics of the oligonucleotide primer.

The invention also provides a genetic marker for HR Conzya canadensis, MOR9, comprising SEQ ID NO: 16. The MOR9 marker comprises an open reading frame comprising the amino acid sequence of SEQ ID NO: 17. The invention comprises a genetic marker having the open reading frame encoding SEQ ID NO: 17, and homologs thereof. As used herein, “homolog” refers to a sequence from Conzya canadensis, other Conzya spp., or another type and species of weed having an amino acid sequence that is at least 70-75% identical to the MOR9 marker having the amino acid sequence of SEQ ID NO: 17. Preferably, the homolog will have a sequence that is at least 75-85% identical to the amino acid sequence of SEQ ID NO: 17. More preferably, the homolog will have a sequence that is at least 85-90% identical to the amino acid sequence of SEQ ID NO: 17. Even more preferably, the homolog will have a sequence that is at least 90-95% identical to the amino acid sequence of SEQ ID NO: 17. Even more preferably, the homolog will have a sequence that is at least 95-99% identical to the amino acid sequence of SEQ ID NO: 17.

Kits of the invention for amplifying at least a portion of a polymorphic marker of herbicide resistance comprise in one or more containers at least one oligonucleotide primer of the invention that anneals to a polymorphic marker of the invention under PCR conditions.

The invention also provides methods to screen for new forms of herbicide agents that are active against genes, their corresponding products and pathways by employing structural information of the genes, the gene products and mutant strains. Positive compounds can then be used as final products or precursors to be further developed into herbicidal agents.

In a further embodiment of the invention, a profile of the isolated markers of the invention are used to as diagnostic tools to identify haplotypes from field isolates of weeds that are associated with HR or HS. DNA is isolated from HR and HS biotypes, and polymorphic markers are isolated in accordance with the methods of the invention. Markers are analyzed for nucleotide structure to identify markers associated with HR or HS. The markers are used to screen field-isolates to HR and HS weeds. Crop management regarding appropriate herbicides is improved by identifying the resistance states of field isolates.

In another embodiment of the invention, a method is provided for screening weeds using polymorphic markers to identify HR and HS biotypes.

In another embodiment of the methods of the invention, plants are exposed to at least one chemical mutagen and seeds are grown in the presence of the herbicide of interest to identify parental plants that have been mutated in a gene(s) or pathways involved in herbicide resistance. Mutant offspring are subject to genormic analysis genes are isolated to serve as diagnostic markers and/or therapeutic agent development.

In an embodiment of the therapeutic agent development, genes involved with herbicide resistance or susceptibility may be used to screen for agents that modify the expression of the gene or its protein product to effect a change in herbicide resistance. For example, but not by way of limitation, a gene conferring herbicide resistance may be targeted with an antisense molecule to decrease the expression of the protein product and thereby interfere with herbicide resistance. In another non-limiting example, the gene conferring herbicide susceptibility may be inserted into an expression vector and expressed in a recombinant cells system. Isolated or purified protein may be contacted with a panel of compounds to determine which compounds bind to the protein. Agents that bind to the protein may be further screened for the ability to interfere with herbicide susceptibility. Such agents may be, but are not limited to small molecules and proteins. Therapeutics may be administered to crop plants to increase their resistance to herbicides while untreated weeds will remain susceptible, for example.

Although several methods of mutagenesis can generate mutant plants, the invention provides methods for generating HS offspring from HR plants. The agents to induce mutagenesis include inhibitors of mismatch repair (MMR), which can lead to as much as a 1000-fold increase in the endogenous DNA mutation rate of a host; the use of chemical agents and their respective analogues such as ethidium bromide, EMS, MNNG, MNU, tamoxifen, 8-hydroxyguanine, as well as others including but not limited to those described in: Khromov-Borisov, N. N., et al. (Mutat. Res. 430:55-74, 1999); Ohe, T., et al. (Mutat. Res. 429:189-199, 1999); Hour, T. C. et al. (1999) Food Chem. Toxicol. 37:569-579; Hrelia, P., et al. (1999) Chem. Biol. Interact. 118:99-111; Garganta, F., et al. (1999) Environ. Mol. Mutagen. 33:75-85; Ukawa-Ishikawa S., et al. (1998) Mutat. Res. 412:99-107); www.ehs.utah.edu/ohh/mutagens. Such agents can be used to further enhance the spectrum of mutations and increase the likelihood of obtaining alterations in one or more genes that can in turn generate naturally occurring HS host weeds from UR parental strains. MMR deficiency leads to hosts with an increased resistance to toxicity by chemicals with DNA damaging activity. Thus, additional genetically diverse hosts can be generated in embodiments of the invention wherein MMR defective plants are exposed to such agents. Generation of such genetically diverse hosts would be otherwise impossible, due to the toxic effects of such chemical mutagens (Colella, G., et al.(1999) Br. J. Cancer 80:338-343; Moreland, N.J., et al. (1999) Cancer Res. 59:2102-2106; Humbert, O., et al. (1999) Carcinogenesis 20:205-214; Glaab, W. E., et al. (1998) Mutat. Res. 398:197-207). Moreover, MMR is responsible for repairing chemical-induced DNA adducts. Therefore, blocking this process would increase the number, types, and rate of mutation and genomic alterations of a weed host (Rasmussen, L. J. et al. (1996) Carcinogenesis 17:2085-2088; Sledziewska-Gojska, E., et al. (1997) Mutat. Res. 383:31-37; and Janion, C. et al. (1989) Mutat. Res. 210:15-22). In addition to the chemicals listed above, other types of DNA mutagens include ionizing radiation and UV-irradiation, which are known to cause DNA mutagenesis can also be used to potentially enhance this process alone or in combination with MMR deficiency.

The HS weed strains described herein have either been generated and characterized in a manner which essentially provides a process by which the manipulation of host genomic DNA of the MR parental line can confer susceptibility against a range of compounds and that these strains are now useful for target discovery and/or therapeutic agent discovery as screening lines.

The above disclosure generally describes the present invention. A more complete understanding can be obtained by reference to the following specific examples that will be provided herein for purposes of illustration only, and are not intended to limit the scope of the invention.

### EXAMPLES

### Example 1

Isolation of DNA Markers for Haplotype Analysis of Weeds for Genomic Identification of Herbicide Resistant Biotypes.

Polymorphic DNA markers are important for mapping the location of genes involved in the biochemical pathway of a given phenotype. Polymorphic markers are useful for the unequivocal identification of weeds that are part of a heterogeneous family that are resistant and susceptible to certain types of herbicides. These markers can be used for the rapid diagnosis of subtypes that are herbicide resistant to certain classes of chemicals to guide farmers on choosing appropriate herbicide management systems for crop management. One such example for using DNA markers to identify HR plants is the isolation of genomic DNA from field specimens of the Conyza canadensis species where approximately 15% of the field populations are naturally resistant to glyphosate, the active ingredient in Roundup® herbicide (VanGessel, M. (2000) “Group G/9 Resistant Horseweed (Conyza canadensis) USA: Delaware” www.weedscience.org/USA.; Robert, S. and U. Baumann (1999) “Resistance to the herbicide glyphosate” Nature 395:25-26; Mountain, W. (1992) Horseweed, Conyza canadensis (L.) Cronq. Regulatory Horticulture, PA Dept. of Agriculture 18(1):31-35). Genomic DNA is isolated from HR and HS Conyza canadensis using DNazol method as described by the manufacturer (Gibco/BRL). Polymorphic markers such as, but not limited to microsatellites, SNPs, and RFLPs can be isolated and used as reagents to identify biotypes of a particular resistance or susceptibility to a class of herbicide.

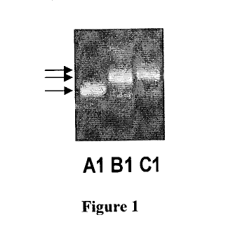

One approach involves the generation of genomic libraries using EcoRI fragments from genomic DNA of the host, which are then subcloned into Lambda Zap cloning vectors and screened for polyA or polyCA nucleotide repeat markers using radiolabelled probes that can identify recombinant clones containing specific repeat markers as previously described (Leach, F. S. et al. (1993) “Mutations of a mutS homolog in hereditary noncolorectal cancer” Cell 75:1215). Positive clones are then isolated and sequenced to identify the nucleotide-specific sequences contained within the flanking regions of the repeat marker. Next, oligonucleotide primers are designed and synthesized for gene-specific identification using the polymerase chain reaction (PCR) as described (Nicolaides, N. C. et al. (1995) “Genomic organization of the human PMS2 gene family” Genomics 30:195). Reactions are carried out using 1 ng of plant DNA as template and the appropriate corresponding primers in 25 ul reactions containing 67 mM Tris, pH 8.8, 16.6 mM (NH₄)₂SO, 6.7 mM MgCl₂, 10 mM 2-mercaptoethanol, 4% DMSO, 1.25 mM each of the four dNTPs, 175 ng of each cDNA specific primer and 1U of Taq polymerase at 94° C. for 30 seconds, 54° C. for 30 seconds and 72° C. for 30 seconds for 30 cycles. Reactions are then added to loading buffer containing 0.05% bromophenol blue plus 10% glycerol and loaded onto 5% METAPHOR agarose gels. Gels are electrophoresed at 150V for 3 hours at 4° C. in tris-acetate running buffer, stained with ethidium bromide and DNA products are visualized by ultraviolet light using a transilluminator. A typical example and result of this procedure is shown in FIG. 1 whereby a novel marker for Glycine max (MOR-117) was isolated using the methods described above from two closely related species that exhibit distinct phenotypes. Marker specific primers were optimized for PCR and genome analysis of three different soybean cultivars (Lane A1: Am strain; Lane B1: Wi82 strain; and Lane C1: CL strain) as described above. As shown in FIG. 1, this method allows for a sensitive analysis of Glycine max that is capable of distinguishing between cultivar strains. Gel analysis measures for species specificity as determined by product formation (indicated by arrow) as well as for cultivar specificity as determined by DNA size. The benefit of using polymorphic repeat markers is that differences in repeat lengths usually occur during natural strain evolution in the wild, allowing for the genetic fingerprint of different strains or cultivars. For MOR-1117, gene sequencing of the marker determined the fragment to contain a 173 bp segment with a polynucleotide repeat consisting of 20 CA-dinucleotides in tandem. Analysis of this marker in the different cultivars (FIG. 1, Lane A1: 169 bp; Lane B1 173 bp; Lane C1 175 bp) demonstrates its ability to identify plant type and cultivar strain. This approach is used for identifying markers in glyphosate resistant (GR) and glyphosate susceptible (GS) Conyza canadensis.

### Example 2

Haplotype Analysis of Heterogeneous Populations of Plants for Diagnosis of Biotypes for Herbicide Resistance.

DNA from the genomes of HR and HS weeds are isolated from field specimens and plated in semisolid medium or in soil treated with active levels of herbicide. Seedlings from plants that are able to grow in the presence of active herbicide levels are classified as herbicide resistant (HR). Those that are not able to grow in the presence of active herbicide levels are classified as herbicide susceptible (HS). DNAs from both classes are analyzed at the genome level using up to ten polymorphic DNA markers to identify haplotype patterns that are associated with susceptibility or resistance using methods described in Example 1. This approach has been used to identify DNA markers in Glycine max and Conyza canadensis for the identification of haplotypes that are associated with certain phenotypes such as but not limited to flower color, herbicide resistance, etc. This approach now serves as a method for distinguishing HR from HS biotypes and is useful for farmers to identify the presence of HR weeds in the crop fields to developing a crop management system prior to planting. The identification of HR associated haplotypes in certain weed species will allow farmers to avoid certain herbicide management systems such as the no-till narrow spacing design used for Roundup Ready® crop plants such as soybeans.

### Example 3

Generation of Glyphosate-Susceptible Conyza canadensis

Naturally occurring HR weeds such as Conyza canadensis are useful for identifying genes that are capable of allowing certain weeds to become resistant to a class of compounds in an attempt to uncover the mechanism(s) of herbicide-resistance. Here we teach the use of employing mutagenesis strategies to glyphosate-resistant (GR) Conyza to generate glyphosate-susceptible (GS) strains that are useful for gene discovery. Briefly, GR Conyza canadensis seedlings are exposed to mutagens such as, but not limited to, mismatch repair inhibitors, chemical mutagens, radiation, etc., and seeds are plated on to solid Murashige and Skoog (MS) media in 150 mm dishes with and without active levels of herbicide. One such example is the use of the small molecule inhibitor of mismatch repair called Morphocene™, and other chemical inhibitors of mismatch repair as described in PCT Publication No. WO 02/054856 (which is incorporated herein by reference). Morphocene™ has been demonstrated to block the endogenous mismatch repair machinery of plants, including Arabidopsis thaliana and Glycine max, leading to genome wide mutations and the production of offspring with new phenotypes. After treatment, one hundred seedlings are moved to two-gallon pots containing metromix 200 soil (Scotts-Sierra Horticultural Products Company, Marysville, Ohio). Plants are grown to maturity (referred to as founder plants) in growth rooms for 12-16 weeks, a time at which Conyza mature and produce seeds. Each plant is capable of producing 200,000 to 300,000 seeds. Seeds are harvested separately from each plant and stored in 4° C. dessicators. Roughly 20,000 seeds from each founder plant is plated onto growth plates containing optimal levels of glyphosate as determined by titration curves. Seedlings are scored glyphosate-susceptible if any of the following features contrast with the parental plant: bleaching (loss of chlorophyll coloration), stunted root formation, or stunted shoot height. Mutant plants are traced back to the appropriate founder and expanded to produce glyphosate-susceptible (GS) offspring. GS plants are then analyzed using a variety of gene expression methods to identify genes whose expression is altered or through standard gene mapping methods using DNA markers to map loci that are linked to the resistant or susceptible phenotypes. The demonstrated ability to generate glyphosate-susceptible Conyza from naturally glyphosate-resistant parental plants allows for the generation of subtypes that can be analyzed by comparative genetics to identify altered gene(s) that confer glyphosate-resistance. This is approach offers certain advantages over methods that employ mutagenesis to GS wild-type strains to identify those that are GR. The generation of GS offspring from GR offspring is now used to identify altered genes responsible for conferring GS from GR parental strains.

Discussion: The results described above will lead to several conclusions. The identification of genomic markers in heterogeneous weed species consisting of subsets that are susceptible and resistant to certain herbicides are useful for identifying HR weeds at the genomic level for aiding in crop management decisions. The use of breeding herbicide resistant and susceptible forms can be used to identify linked genetic loci to identify genes involved to herbicide resistance. The mutagenesis of naturally occurring herbicide resistant weeds are useful for identifying genes involved in resistance to a certain class of compound.

### Example 4

Isolation of DNA Markers for Haplotype Analysis of Horseweeds for Genomic Identification of Herbicide Resistant Biotypes

Isolation and modification of the genomic DNA: Total horseweed (Conyza Canadensis) genomic DNA was isolated from 100 mg of leaves using Plant DNAZol as described by the manufacturer (Invitrogen). The typical yield was 10-20 μg DNA per preparation. Two different biotypes of horseweed were used as source of the DNA and were designated HR and HS. HR has been confirmed to exhibit glyphosate tolerance while HS is sensitive to glyphosate treatment.

DNAs were digested with two restriction enzymes, and the resulting DNA fragments were ligated with adapters simultaneously in buffer (50 mM Tris-HCl, 10 mM MgCl₂, 10 mM DTT, 1 mM ATP, pH 7.5, 50 mM NaCl, 45 μg/ml BSA). 10 units of each restriction enzyme were used to digest 2.5 μg DNA. 1.5 units of T4 DNA ligase, 2-10 pmoles of Adapter 1 (for one restriction enzyme) and 2-10 pmoles of Adapter 2 (for second restriction enzyme) were also added. Incubation was performed in the total volume of 22 μl at 37° C. for 2 hr.

Preparation of adapters: The following are the sequences of the adapters:

| | | |
|---|---|---|
| Adapter 1 | 5′-GACGATGAGTCCTGAG-3′ | (SEQ ID NO:1) |
| | 3′-TACTCAGGACTCAT-5′ | (SEQ ID NO:2) |
| Adapter 2 | 5′-CTCGTAGACTGCGTACC-3′ | (SEQ ID NO:3) |
| | 3′-CATCTGACGCATGGTTAA-5′ | (SEQ ID NO:4) |

To prepare 50 pmoles/μl of Adapter 1, equal volume of 100 μM of the 16-mer 5′-GACGATGAGTCCTGAG-3′ SEQ ID NO: 1) and the 14-mer 5′-TACTCAGGACTCAT-3′ SEQ ID NO: 2) were mixed, incubated at 85° C. for 5 min. and slowly cooled to room temperature. 50 pmoles/μl of Adapter 2 was prepared in the same way by mixing the 16-mer 5′-CTCGTAGACTGCGTACC-3′ SEQ ID NO: 3) and 17-mer 5′-AATTGGTACGCAGTCTAC-3′ SEQ ID NO: 4).

The DNAs modified as above were used for two rounds of PCR (pre-amplification and selective amplification).

Pre-amplification of fragments: In pre-amplification, primers 2-0 and 1-C dissolved at 10 mM in ddH₂O were used.

| | | |
|---|---|---|
| 2-0 primer: | 5′-CTCGTAGACTGCGTACCAATTC | (SEQ ID NO:5) |
| 1-C primer: | 5′-GACGATGAGTCCTGAGTACC | (SEQ ID NO:6) |

The reaction was performed as follows: 2.5 μl of ½ dilution of modified DNA template, 10 mM Tris-HCl (pH 8.3), 50 mM KCl, 2 mM MgCl₂, 0.001% gelatin, 200 μM dNTPs, 6 pmoles of primers 2-0 and 1-C, 0.5 units of Taq DNA polymerase in the total volume of 20 μl.

The amplification was performed in Hybaid Omni thermal cycler. The cycle profile was as follows:

| | | |
|---|---|---|
| 1 cycle: | denaturation: | 94° C., 2 min |
| 20 cycles: | denaturation: | 94° C., 20 seconds |
| | annealing: | 56° C., 30 seconds |
| | extension: | 72° C., 2 min |
| 1 cycle: | | 72° C., 2 min |
| 1 cycle: | | 60° C., 30 min |

Selective amplification of fragments: Selective amplification was performed as follows: 5 μl of 1/20 dilution of preamplification product, 10 mM Tris-HCl (pH 8.3), 50 mM KCl, 2 mM MgCl₂, 0.001% gelatin, 200 μM dNTPs, 6 pmoles of primer 2-TG (labeled with fluorescence dye) and primer 1-CAC, 0.5 units of Taq DNA polymerase in the total volume of 20 μl.

| | |
|---|---|
| 2-TG primer: | |
| 5′-CTCGTAGACTGCGTACCAATTCTG-3′ | (SEQ ID NO:7) |
| 1-CAC primer: | |
| 5′-GACGATGAGTCCTGAGTACCAC-3′ | (SEQ ID NO:8) |

The amplification was performed in Hybaid Omni thermal cycler. The cycle profile was as follows:

| | | |
|---|---|---|
| 1 cycle: | denaturation: | 94° C., 2 min |
| 10 cycles: | denaturation: | 94° C., 20 seconds |
| | annealing: | 66° C., 30 seconds (decrease 1° C./cylce) |
| | extension: | 72° C., 2 min |
| 20 cycles: | denaturation: | 94° C., 20 seconds |
| | annealing: | 56° C., 30 seconds |
| | extension: | 72° C., 2 min |
| 1 cycle: | | 60° C., 30 min |

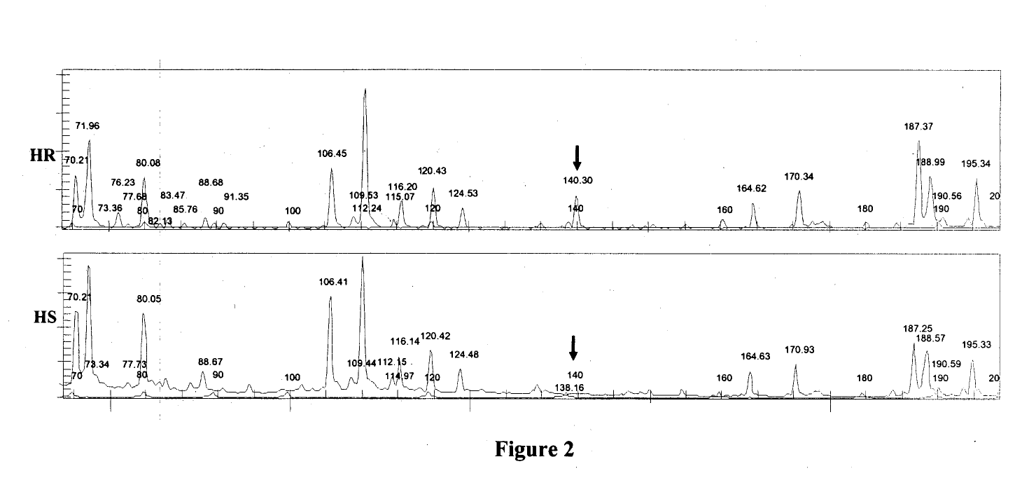

Gel analysis of amplified fragments: After selective amplification, the reaction products were analyzed on Beckman CEQ2000XL sequencing machine. DNA size standard 400 was used for the fragment analysis. 1/50 dilution of DNA size standard was made in sample loading buffer (Deionized formamide), 8 μl of amplified fragments were added to 25 μl of diluted DNA size standard and loaded onto CEQ2000XL with capillary temperature at 50° C., sample denaturation at 90° C. for 2 min injection at 2 kV 30 seconds and separation at 6 kV 35 min. The results of the analysis are shown in FIG. 2. The top panel is the fluorescence histogram of HR DNA and bottom panel is the one for HS DNA. A 140 bp fragment was identified to be present only in HR but absent in HS horseweed genomic DNA (arrow).

Cloning of the Polymorphic Fragment: To clone this fragment, selective amplification was performed as above with the exception of 2-TG primer not being labeled with dye and the products were separated on 20% polyacrylamide gel. The bands with the size around 140 bp were isolated with Qiagen PCR purification kit (Qiagen) and cloned into TA cloning vector (Invitrogen) as described by manufacturer. More than 20 clones were picked and sequenced. The clones with the insert size matching expected 140 bp were chosen and their inserts were used as probes in Southern blot analysis to identify which clone represents the band present in HR but absent in HS. Amplified products from HR and HS were separated on 2.5% agarose gel and transferred onto N-Hybond+ membrane (Amersham) by standard blotting procedures using 20×SSC as the transfer buffer. Each membrane will be incubated at 55° C. in 10 ml of hybridization solution (North2South non-radioactive detection kit, Pierce) containing 100 ng of cloned insert DNA probes, which were generated by PCR amplification and gel purification, and labeled according to the manufacturer's directions. Membranes were washed three times in 2×SSC, 0.1% SDS at 55° C., and three times in 2×SSC at ambient temperature. Detection of hybridized probes was carried out using enhanced chemiluminescence (ECL) and autoradiography.

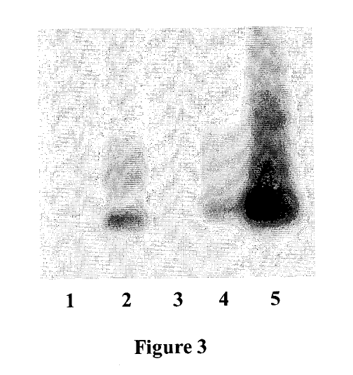

One class of the clones met such criteria and Southern blot analysis is presented in FIG. 3. Lane 1 and 3 are amplified products from HS while lane 2 and 4 are those from HR. Lane 5 is the insert from the clone. Lane 1 and 2 are amplified with dye-labeled 2-TG primer while lane 3 and 4 with 2-TG not labeled with dye. The sequence of this clone was determined.

Determination of Flanking Sequences of the Polymorphic Fragment: To obtain the sequences upstream and downstream of this fragment in the genome, the thermal asymmetric interlaced (TAIL) PCR method was performed on HR genomic DNA as described by Liu et al. (1995) The Plant Journal 8(3):457-463). The primers used were:

| | |
|---|---|
| AD3 (degenerate primer): | |
| WGTGNAGWANCANAGA | (SEQ ID NO:9) |
| 1-F: | |
| 5′-CACATCTTTAGCATCGGC-3 | (SEQ ID NO:10) |
| 2-F: | |
| 5′-AAAGTGGCTGAATCGTGG-3′ | (SEQ ID NO:11) |
| 3-F: | |
| 5′-GATTTGAATGGTGGTGCC-3′ | (SEQ ID NO:12) |
| 1-R: | |
| 5′-GGCACCACCATTCAAATC-3′ | (SEQ ID NO:13) |
| 2-R: | |
| 5′-ATACCACGATTCAGCCAC-3′ | (SEQ ID NO:14) |
| 3-R: | |
| 5′-GCCGATGCTAAAGATGTG-3′ | (SEQ ID NO:15) |

TAIL-PCR products were cloned into TA cloning vector (Invitrogen) and sequenced to obtain the flanking sequences of the polymorphic fragment.

Assembly of the DNA Marker: A contig was assembled from all three sequences. The presence of this contig was confirmed by PCR using HR DNA as template and two primers spanning the three fragments in the contig. Interestingly, no product was amplified using these same two primers with HS DNA as the template. The sequence of this contig is shown in SEQ ID NO: 16. The contig encodes a protein of unknown nature (partial open reading frame without stop codon) (shown in SEQ ID NO: 17). The polymorphic marker was designated MOR9.

### Example 5

Haplotype Analysis of Heterogeneous Populations of Plants for Diagnosis of Biotypes for Herbicide Resistance

Design of Primers for Diagnosis with the DNA Marker: PCR Primer 1 (SEQ ID NO: 18) and 2 (SEQ ID NO: 19) were designed based on the sequence of the DNA marker obtained in Example 4. Control PCR Primer 1 (SEQ ID NO: 20) and 2 (SEQ ID NO: 21) are primers that will amplify a fragment in all horseweed biotypes.

| | |
|---|---|
| PCR Primer 1: | |
| 5′-TTG TCG CTG TCC AAC CAT TG-3′ | (SEQ ID NO:18) |
| PCR Primer 2: | |
| 5′-TTG GCA TGG TCT GTA GCT GG-3′ | (SEQ ID NO:19) |
| Control PCR Primer 1: | |
| 5′-CCA TCG TAT CAT CAT GTG C-3′ | (SEQ ID NO:20) |
| Control PCR Primer 2: | |
| 5′-TGC AAT ATG TTA AAG TAG AGC-3′ | (SEQ ID NO:21) |

Analysis of the DNA Marker: Horseweeds were either germinated from seeds or seedlings were transferred from the field into the greenhouse. DNAs from the genomes of HR and HS horseweeds were isolated. At the same time, glyphosate was applied at 2 lb active ingredient/acre rate to confirm the plants' sensitivity to the herbicide.

Using HR or HS genomic DNAs as templates, PCR amplification was performed in 67 mM Tris pH 8.8, 16.6 mM NH₄ SO₄, 6.7 mM MgCl₂, 100 mM β-mercaptoethanol, 6.7 μM EDTA pH 8.0, 6% DMSO, 1.25 mM dNTPs and 1.25 units Taq polymerase with the total volume of 25 μl and cycled at 1×94° C., 2 min; 45×94° C., 30 sec; 56° C. 30 sec; 72° C., 1.5 min; 1×72° C., 10 min. For the MOR9 marker assay, 20 pmoles of PCR Primer 1 (SEQ ID NO: 18) and PCR Primer 2 (SEQ ID NO: 19) were used while Control PCR Primer 1 (SEQ ID NO: 20) and Control PCR Primer 2 (SEQ ID NO: 21) were used in control experiment.

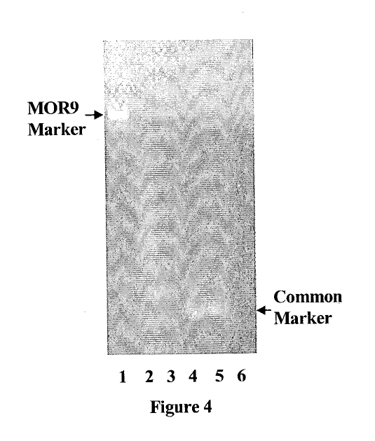

A typical result is shown in FIG. 4. Lanes 1-3 are the amplification products for the MOR9 marker, while lanes 4-6 are the amplification products for control marker. The templates for lanes 1 and 4 are DNA from glyphosate-tolerant horseweed, the templates for lanes 2 and 5 are from glyphosate-sensitive horseweed, and the templates for lanes 3 and 6 are water controls (i.e., without any DNA).

In total, 30 biotypes (7 HS and 23 HR) were analyzed using 74 HR and 32 HS plants. There is 100% correlation of the absence of MOR9 marker with HS and 88% correlation of the presence with HR (Table 1).

TABLE 1

| Horseweed | Glyphosate | Common | MOR9 | |
|---|---|---|---|---|
| Biotype | Resistance | PCR Marker | Marker | Correlation |
| 13D | + | + | + | + |
| 2D | + | + | + | + |
| 3M | + | + | + | + |
| 25D | + | + | + | + |
| 18D | + | + | + | + |
| 12M | + | + | + | + |
| 17D | + | + | + | + |
| 19M | + | + | + | + |
| 16M | + | + | + | + |
| 15D | + | + | + | + |
| 27 | + | + | + | + |
| 14M | + | + | + | + |
| 9D | + | + | + | + |
| 22M | + | + | + | + |
| 5M | + | + | + | + |
| 10D | + | + | − | − |
| 11D | + | + | − | − |
| 4M | − | + | − | + |
| 28 | − | + | − | + |
| Home | − | + | − | + |
| HR | − | + | − | + |
| WF | − | + | − | + |
| WS | − | + | − | + |
| MT | − | + | − | + |

### Example 5

Homologs of MOR9:

Homolog 1: Two homologs of MOR9 clone were identified. Homolog 1 was cloned by regular PCR with two primers. During the diagnostic assay of the MOR9 marker, a pair of primers was shown to amplify a 281 bp product with the genomic DNA from glyphosate-sensitive and tolerant horseweed. The PCR condition was the same as that used to amplify MOR9 in the diagnostic assay in Example 4. The sequences of the two primers are:

| | | |
|---|---|---|
| Primer 1: | 5′-CACATCTTTAGCATCGGC-3′ | (SEQ ID NO:62) |
| Primer 2: | 5′-TCATTCGGAGAAACATCATG-3′ | (SEQ ID NO:63) |

The 281 bp PCR products were sequenced and shown to be the same in both glyphosate-sensitive and tolerant horseweed. The sequence showed significant homology (>62%) to MOR9 DNA marker and was named as MOR9 Homolog 1 (MOR9 H1). The nucleotide sequence of MOR9 H1 is shown in SEQ ID NO: 60. An alignment of the overlapping regions of MOR9 (SEQ ID NO: 126) and MOR9 H1 (SEQ ID NO: 127) is as follows (differences in the sequences are noted in boldface):

| | |
|---|---|
| MOR9 Marker | CACATCTTTA GCATCGGCCA CCATTGAAAA AGTGGCTGAA TCGTGGTATA |
| MOR9 H1 | CACATCTTTA GCATCGGCCA CCATTGAAAA AGTGGCTGAA TCATGGGATA |
| | 51 100 |
| MOR9 Marker | AGAATGTTGT ATTGCAGGTT GATGTTGAGA GGGATTTGGA TGATTTGAAT |
| MOR9 H1 | AAAATGTCGC TACAAGTGTT GATGATGGTA GGGACTTGAA TGATTCTAAT |
| | 101 150 |
| MOR9 Marker | GGTGGTG.CC AGAATTCTAC TGCTGAGTCA TCTTTGCATG ATTTCCATGC |
| MOR9 H1 | GGTGATGGCC TTCACTCGAC TGTTGAACCA ACATTGCGTG GTTTGCATGC |
| | 151 200 |
| MOR9 Marker | AAAAGGTGGT GCTACTCATG TTTCCCCTAT GCTTGATCCT CCTAAGTTTC |
| MOR9 H1 | ATATGTTGGT GATTCTAATG TACCTCCAA. .A.....C.. .CAAAGTTCC |
| | 201 250 |
| MOR9 Marker | CTCCTGGTAC TACTTATTTT AAGCCAGCTA CAGACACATG CCAATGACAT |
| MOR9 H1 | CTCCTGATGC TTCTTATTTT CAACCGGCTG CATGT.CATG CAAATGACAT |
| | 251 298 |
| MOR9 Marker | TCTTGATGTT .......... .......... .......... ........ |
| MOR9 H1 | TCACCCTGCT ACAGATGAGG CCCCTTTGCA TGATGTTTCT CCGAATGA |

Homolog 2: A second homolog of MOR9 was discovered by RT-PCR. First, RNA was extracted from horseweed (glyphosate susceptible or resistant) and reverse transcribed with adapter-T25VN (AAG CAG TGG TAT CAA CGC AGA GTA CTT TTT TTT TTT TTT TTT TTT TTT TTV N) (SEQ ID NO: 64) primer under standard RT conditions. HWMOR 9-RACE1F primer (CAC ATC TTT AGC ATC GGC CAC CAT TG) (SEQ ID NO: 65), primer CTA ATA CGA CTC ACT ATA GGG CAA GCA GTG GTA TCA ACG CAG AGT (SEQ ID NO: 66) and primer CTA ATA CGA CTC ACT ATA GGG C (SEQ ID NO: 67) were used to amplify from reverse transcribed product under the following amplification conditions:

| | |
|---|---|
| 5 cycles: | 94° C., 30 sec 72° C., 3 min |
| 5 cycles: | 94° C., 30 sec 70° C., 30 sec 72° C., 3 min decrease |
| | (0.5° C./cycle) |
| 25 cycles: | 94° C., 30 sec 68° C., 30 sec; 72° C., 3 min |

A second round of PCR was performed on diluted primary PCR product with HWMOR9-FACE2F (GTG GCT GAA TCG TGG TAT AAG AAT G) (SEQ ID NO: 68) and nested primer (AAG CAG TGG TAT CAA CGC AGA GT) (SEQ ID NO: 69) under the following amplification conditions:

20 cycles: 94° C., 30 sec; 68° C., 30 sec; 72° C., 3 min.

A PCR product was found and sequenced that also showed homology to MOR9 and MOR9 H1. The second homolog was named MOR9 Homolog 2 (MOR9 H2) and the nucleic acid sequence of MOR9 H2 is shown in SEQ ID NO: 61. An alignment of the overlapping regions of MOR9 (SEQ ID NO: 126) and the MOR9 H1 (SEQ ID NO: 127) and MOR9 H2 (SEQ ID NO: 128) is as follows (differences between the homologs and MOR9 are shown in boldface; differences between the homologs are indicated by an underline):

| | |
|---|---|
| | 1 50 |
| MOR9 | CACATCTTTA GCATCGGCCA CCATTGAAAA AGTGGCTGAA TCGTGGTATA |
| MOR9 H1 | CACATCTTTA GCATCGGCCA CCATTGAAAA AGTGGCTGAA TCATGGGATA |
| MOR9 H2 | .......... .......... .......... .......... .......... |
| | 51 100 |
| MOR9 | AGAATGTTGT ATTGCAGGTT GATGTTGAGA GGGATTTGGA TGATTTGAAT |
| MOR9 H1 | AAAATGTCGC TACAAGTGTT GATGATGGTA GGGACTTGAA TGATTCTAAT |
| MOR9 H2 | .......... .......... ....ATGGTA GGGATTTGAA TGATTCGACT |
| | 101 150 |
| MOR9 | GGTGGTG.CC AGAATTCTAC TGCTGAGTCA TCTTTGCATG ATTTCCATGC |
| MOR9 H1 | GGTGATGGCC TTCACTCGAC TGTTGAACCA ACATTGCGTG GTTTGCATGC |
| MOR9 H2 | GGGGATGGCT TACACTCGAC TGCTGAACCA ACATTGCATG GTTTGCATGC |
| | 151 200 |
| MOR9 | AAAAGGTGGT GCTACTCATG TTTCCCCTAT GCTTGATCCT CCTAAGTTTC |
| MOR9 H1 | ATATGTTGGT GATTCTAATG TACCTCCAA. .A.....C.. .CAAAGTTCC |
| MOR9 H2 | AAATGTTGAT GATTGTACTG TGCCTCCTAT GCCGGAACCG CCAAAGTTCC |
| | 201 250 |
| MOR9 | CTCCTGGTAC TACTTATTTT AAGCCAGCTA CAGACACATG CCAATGACAT |
| MOR9 H1 | CTCCTGATGC TTCTTATTTT CAACCGGCTG CATGT.CATG CAAATGACAT |
| MOR9 H2 | CTCCTGATGC TACTTACTTT CAGCCGGCTG CATGT.CATG TAAATGACAT |
| | 251 300 |
| MOR9 | TCTTGATGTT .......... .......... .......... .......... |
| MOR9 H1 | TCACCCTGCT ACAGATGAGG CCCCTT.TGC ATGATGTTTC TCCGAATGA. |
| MOR9 H2 | TCATCCTGCT TCACATGAGG CCCCTTATGC ATGATGTTAC TCCTAATGAT |
| | 301 350 |
| MOR9 | .......... .......... .......... .......... .......... |
| MOR9 H1 | .......... .......... .......... .......... .......... |
| MOR9 H2 | CTTAGTGGAT ACCCTGACAG TCCTAAGGTC CAGCAGCCGC GTACTTATGC |
| | 351 400 |
| MOR9 | .......... .......... .......... .......... .......... |
| MOR9 H1 | .......... .......... .......... .......... .......... |
| MOR9 H2 | TTCTATCTTT CAGGATGCGG CTAACATCAA CAAGAAAGGT AAATTGAGAT |
| | 401 423 |
| MOR9 | .......... .......... ... |
| MOR9 H1 | .......... .......... ... |
| MOR9 H2 | TCATCCCTCC AAAAAAAAAA AAA |

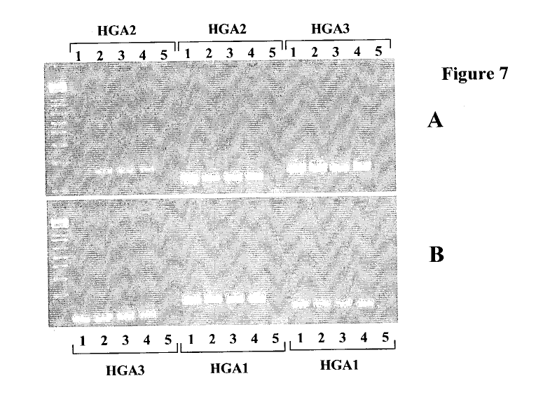

A pair of primers was designed and used for PCR amplification of both glyphosate susceptible and glyphosate resistant horseweed. The result was shown in FIG. 7 which indicates that MOR9 H2 is present in both biotypes.

### Example 6

Cloning of the GA repeat from horseweed: Adapter-ligated PCR was used to identify GA repeat sequences from horseweed. Briefly, genomic DNA was extracted as described in Example 4 and digested with either a single restriction enzyme or a combination of enzymes. Then a mixture of primer adapters for the restriction enzymes recognition sequences (for single enzyme digestion or combination enzyme digestion) were ligated to restricted DNA fragments. Sequence of the primers used was as follows:

| | |
|---|---|
| A primer: | |
| 5′-GTAATACGACTCACTATAGGGCACGCG- | (SEQ ID NO:70) |
| TGGTCGACGGCCCGGGCTGGT-3′ | |
| B primer: | |
| 5′-AATTACCAGCCC-NH2 | (SEQ ID NO:71) |
| C primer: | |
| 5′-GATCACCAGCCC-NH2 | (SEQ ID NO:72) |
| D primer: | |
| 5′-AGCTACCAGCCC-NH2 | (SEQ ID NO:73) |

The ligation products were used as templates for primary PCR amplification with the following primer: AP1 and GAGB or AP1 and GAH.

| | | |
|---|---|---|
| AP1: | 5′-GTAATACGACTCACTATAGGGC-3′ | (SEQ ID NO:74) |
| GAGB: | 5′-GAGAGAGAGAGAGAGAGAGAGB-3′ | (SEQ ID NO:75) |
| GAH: | 5′-GAGAGAGAGAGAGAGAGAGAH-3′ | (SEQ ID NO:76) |

The primary PCR condition is as follows:

| | |
|---|---|
| 5 cycles: | 94° C., 30 seconds; 65° C., (decrease 1° C., after each cycle) |
| | 30 seconds, 72° C., 3 minutes |
| 40 cycles: | 94° C., 30 seconds, 60° C., 30 seconds, 72° C. 3 minutes |
| 1 cycle: | 72° C., 10 minutes. |

The primary PCR product was diluted 1/50 with water and used at 1/1000 for secondary PCR amplification which used primer AP2 (SEQ ID NO: 77) and GAGB (SEQ ID NO: 75) or AP2 (SEQ ID NO: 77) and GAH (SEQ ID NO: 76). The sequence of Primer AP2 is as follows:

AP2: ACTATAGGGCACGCGTGGT (SEQ ID NO: 77)

The secondary PCR products were cloned into TA cloning vector (Invitrogen) and sequences downstream of GA repeats were determined.

To identify the sequences upstream of GA repeat, two nested primers (NP1 and NP2) for each of the HGA clones based on the determined downstream sequences were designed and used to repeat the primary PCR with AP1 and NP1 as above using the same ligation products. The sequences of the specific NP1 and NP2 primers is as follows:

| | |
|---|---|
| NP1-HGA1: | |
| 5′-CCATCGTATCATCATGTGC-3′ | (SEQ ID NO:113) |
| NP2-HGA1: | |
| 5′-TAGCTTGCAAAAGTTCTG-3′ | (SEQ ID NO:114) |
| NP1-HGA2: | |
| 5′-TACCAATATTGCCCTTGG-3′ | (SEQ ID NO:116) |
| NP2-HGA2: | |
| 5′-GTATACCCTTTTCCGTTCC-3′ | (SEQ ID NO:117) |
| NP1-HGA3: | |
| 5′-TACCCAACCCTATCTFFCC-3′ | (SEQ ID NO:119) |
| NP2-HGA3: | |
| 5′-TCCATTCATTCTTCACCC-3′ | (SEQ ID NO:120) |
| NP1-HGA4: | |
| 5′-ATGTTAGTGTTCTACACC-3′ | (SEQ ID NO:122) |
| NP2-HGA4: | |
| 5′-CTTAGATACGTAACAACC-3′ | (SEQ ID NO:123) |
| NP1-HGA5: | |
| 5′-AACGACTCTTCCAAACCC-3′ | (SEQ ID NO:124) |
| NP2-HGA5: | |
| 5′-TGACCTCAATTGACTTGC-3′ | (SEQ ID NO:125) |

Then a secondary PCR amplification was performed with AP2 (SEQ ID NO: 77) and NP2 primers. The final products were cloned and sequenced to determine the upstream sequence of a particular clone on which the NP1 and NP2 was based. The sequences of upstream and downstream regions were assembled into one contig and used for designing primers to amplify the simple sequence repeat (SSR) marker.

Five complete markers were assembled and their sequences are HGA1 (SEQ ID NO: 78); HGA2 (SEQ ID NO: 79); HGA3 (SEQ ID NO: 80); HGA4 (SEQ ID NO: 81); HGA5 (SEQ ID NO: 82).

Primers were designed to assay for polymorphisms between different biotypes of horseweed for each of the HGA sequences. Examples of sequences of diagnostic primers for HGA sequences are as follows:

| | |
|---|---|
| D-HGA1: | |
| 5′-TGCAATATGTTAAAGTAGAGC-3′ | (SEQ ID NO:115) |
| D-HGA2: | |
| 5′-TTCATGGTGATGACTCGGCAGC3′ | (SEQ ID NO:118) |
| D-HGA3: | |
| 5′-CCATAATTTGGTGTAAGAATC-3′ | (SEQ ID NO:121) |
| D-HGA5: | |
| 5′-ATATAGACATCCATTCCA-3′ | (SEQ ID NO:126) |

Amplifications were performed using the diagnostic primers for the GHA sequences with either the NP1 primers or NP2 primers. No polymorphisms were found when assaying for the HGA1, HGA2, or HGA3 markers using the four different horseweed collections available (FIG. 7).

What is claimed is:

1. A method for identifying polymorphic markers of herbicide resistance in a plant comprising:
(a) isolating genomic DNA from an herbicide susceptible plant and an herbicide resistant plant of the same species;
(b) performing genetic analysis on said genomic DNA of said an herbicide susceptible plant and said herbicide resistant plant; and
(c) identifying differences between the genomic DNA of said herbicide susceptible plant and said herbicide resistant plant,
(d) identifying said differences that correlate with herbicide resistance or herbicide susceptibility by screening samples of herbicide resistant and herbicide susceptible plants;
thereby identifying polymorphic markers of herbicide resistance in said plant.

2. The method of claim 1 wherein said polymorphic markers comprise polynucleotide microsatellite markers where herbicide resistant plants have a distinct haplotype pattern in comparison to herbicide susceptible species.

3. The method of claim 1 wherein said plant is Conyza canadensis.

4. The method of claim 1 wherein said plant is Lolium rigidum.

5. The method of claim 1 wherein said plant is a goosegrass species.

6. The method of claim 1 wherein said herbicide comprises glyphosate.

7. The method of claim 1 wherein said herbicide comprises paraquot.

8. The method of claim 1 wherein said herbicide comprises sulfonyl urea moities.

9. A method for generating herbicide susceptible weeds from herbicide resistant weeds comprising:
(a) mutagenizing said resistant weeds, thereby creating mutant parental weeds;
(b) testing progeny of said mutant parental weeds for susceptibility to said herbicide; and
(c) selecting said mutant parental weeds producing herbicide susceptible progeny.

10. The method of claim 9 wherein the step of testing comprises analyzing said progeny for resistance to an herbicide selected from the group consisting of aminoglycosides, 5-enolpyruvylshikimate-3-phosphate synthase inhibitors, triazine-based herbicides, beta-lactams, macrolides, lincosamides, sulfonamides, atrazine, alachlor, isoniazids, and metribuzin.

11. The method of claim 9 wherein said mutagenizing is accomplished by introducing into said herbicide resistant weed a dominant negative allele of a mismatch repair gene.

12. The method of claim 11 wherein said dominant negative allele of a mismatch gene is a dominant negative allele of a gene encoding a mismatch repair protein selected from the group consisting of PMS2, PMS1, MLH1, MSH2, MSH3, MSH6, MSH7, MSH6-1, PMSR2, PMSR3, and PMSL9.

13. The method of claim 12 wherein said dominant negative allele is a PMS2 truncation mutant.

14. The method of claim 13 wherein said truncation mutant encodes PMS2-134.

15. The method of claim 9 wherein said mutagenizing is accomplished by introducing into said herbicide resistant weed a chemical inhibitor of mismatch repair selected from the group consisting of an anthracene, an ATPase inhibitor, a nuclease inhibitor, a polymerase inhibitor and an antisense oligonucleotide that specifically hybridizes to a nucleotide encoding a mismatch repair protein dominant negative allele of a mismatch repair gene.

16. The method of claim 15 wherein said chemical inhibitor is an anthracene having the formula: 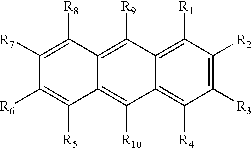
wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein said heteroalkyl, heteroaryl, and substituted heteroaryl contain at least one heteroatom that is oxygen, sulfur, a metal atom, phosphorus, silicon or nitrogen; and wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein said amino groups are optionally substituted with an acyl group, or 1 to 3 aryl or lower alkyl groups.

17. The method of claim 16 wherein R₅ and R₆ are hydrogen.

18. The method of claim 16 wherein R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

19. The method of claim 16 wherein said chemical inhibitor of mismatch repair is selected from the group consisting of 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

20. The method of claim 9 wherein said mutagenizing is accomplished using T-DNA insertional mutagenesis.

21. A method for generating herbicide resistant weeds from herbicide susceptible weeds comprising:
(a) mutagenizing said susceptible weeds, thereby creating mutant parental weeds;
(b) testing progeny of said mutant parental weeds for resistance to said herbicide; and
(c) selecting said mutant parental weeds producing herbicide resistant progeny.

22. The method of claim 21 wherein the step of testing comprises analyzing said progeny for susceptibility to an herbicide selected from the group consisting of aminoglycosides, 5-enolpyruvylshikimate-3-phosphate synthase inhibitors, triazine-based herbicides, beta-lactams, macrolides, lincosamides, sulfonamides, atrazine, alachlor, isoniazids, and metribuzin.

23. The method of claim 21 wherein said mutagenizing is accomplished by introducing into said herbicide resistant weed a dominant negative allele of a mismatch repair gene.

24. The method of claim 23 wherein said dominant negative allele of a mismatch gene is a dominant negative allele of a gene encoding a mismatch repair gene selected from the group consisting of PMS2, PMS1, MLH1, MSH2, MSH3, MSH6-1, MSH7, MSH6, PMSR2, PMSR3, and PMSL9.

25. The method of claim 24 wherein said dominant negative allele is a PMS2 truncation mutant.

26. The method of claim 25 wherein said truncation mutant encodes PMS2-134.

27. The method of claim 21 wherein said mutagenizing is accomplished by introducing into said herbicide resistant weed a chemical inhibitor of mismatch repair selected from the group consisting of an anthracene, an ATPase inhibitor, a nuclease inhibitor, a polymerase inhibitor and an antisense oligonucleotide that specifically hybridizes to a nucleotide encoding a mismatch repair protein dominant negative allele of a mismatch repair gene.

28. The method of claim 27 wherein said chemical inhibitor is an anthracene having the formula: 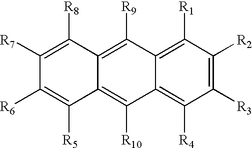
wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein said heteroalkyl, heteroaryl, and substituted heteroaryl contain at least one heteroatom that is oxygen, sulfur, a metal atom, phosphorus, silicon or nitrogen; and wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein said amino groups are optionally substituted with an acyl group, or 1 to 3 aryl or lower alkyl groups.

29. The method of claim 28 wherein R₅ and R₆ are hydrogen.

30. The method of claim 28 wherein R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

31. The method of claim 28 wherein said chemical inhibitor of mismatch repair is selected from the group consisting of 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

32. The method of claim 21 wherein said mutagenizing is accomplished using T-DNA insertional mutagenesis.

33. A method for identifying a mutant gene conferring herbicide resistance comprising
(a) comparing the genome of a naturally occurring herbicide resistant plant to the genome of an herbicide susceptible plant;
(b) determining genetic differences between said herbicide resistant plant to the herbicide susceptible plant; and
(c) sequencing a region of DNA comprising said genetic difference.

34. The method of claim 33 wherein said genome of said herbicide resistant plant and said genome of said herbicide susceptible plant are compared by a technique selected from the group consisting of microarray analysis, genotyping of repetitive sequences using microsatellite markers to identify linked genomic segments that are associated with a particular trait, single nucleotide polymorphic (SNP) analysis, restriction fragment length polymorphism (RFLP) analysis, amplified fragment length polymorphism (AFLP) analysis, simple sequence length polymorphism analysis (SSLPs), randomly amplified polymorphic DNAs (RAPDs), DNA amplification fingerprinting (DAF), sequence characterized amplified regions (SCARs), arbitrary primed polymerase chain reaction (AP-PCR), and single nucleotide polymorphisms (SNPs).

35. A method for identifying a mutant gene conferring herbicide resistance comprising introducing into an herbicide susceptible weed gene fragments from an herbicide resistant weed, thereby creating a transfected herbicide susceptible strain;
(a) screening progeny of said transfected herbicide susceptible strain for herbicide resistance; and
(b) sequencing said gene fragment to identify an herbicide resistance gene.

36. The method of claim 35 wherein said genome of said herbicide resistant plant and said genome of said herbicide susceptible plant are compared by a technique selected from the group consisting of microarray analysis, genotyping of repetitive sequences using microsatellite markers to identify linked genomic segments that are associated with a particular trait, single nucleotide polymorphic (SNP) analysis, restriction fragment length polymorphism (RFLP) analysis, amplified fragment length polymorphism (AFLP) analysis, simple sequence length polymorphism analysis (SSLPs), randomly amplified polymorphic DNAs (RAPDs), DNA amplification fingerprinting (DAF), sequence characterized amplified regions (SCARs), arbitrary primed polymerase chain reaction (AP-PCR), and single nucleotide polymorphisms (SNPs).

37. A method for identifying a mutant gene conferring herbicide susceptibility comprising
(a) introducing into an herbicide resistant weed gene fragments from an herbicide susceptible weed, thereby creating a transfected herbicide resistant strain;
(b) screening progeny of said transfected herbicide resistant strain for herbicide susceptibility; and
(c) sequencing said gene fragment to identify an herbicide susceptibility gene.

38. The method of claim 37 wherein said genome of said herbicide resistant plant and said genome of said herbicide susceptible plant are compared by a technique selected from the group consisting of microarray analysis, genotyping of repetitive sequences using microsatellite markers to identify linked genomic segments that are associated with a particular trait, single nucleotide polymorphic (SNP) analysis, restriction fragment length polymorphism (RFLP) analysis, amplified fragment length polymorphism (AFLP) analysis, simple sequence length polymorphism analysis (SSLPs), randomly amplified polymorphic DNAs (RAPDs), DNA amplification fingerprinting (DAF), sequence characterized amplified regions (SCARs), arbitrary primed polymerase chain reaction (AP-PCR), and single nucleotide polymorphisms (SNPs).

39. A method for identifying a mutant gene conferring herbicide susceptibility comprising
(a) crossing an herbicide resistant weed with an herbicide susceptible weed, thereby creating a crossed strain;
(b) screening progeny for herbicide susceptibility; and
(c) performing genetic analysis on said crossed strain producing herbicide susceptible progeny to identify an herbicide susceptibility gene.

40. The method of claim 39 wherein said genome of said herbicide resistant plant and said genome of said herbicide susceptible plant are compared by a technique selected from the group consisting of microarray analysis, genotyping of repetitive sequences using microsatellite markers to identify linked genomic segments that are associated with a particular trait, single nucleotide polymorphic (SNP) analysis, restriction fragment length polymorphism (RFLP) analysis, amplified fragment length polymorphism (AFLP) analysis, simple sequence length polymorphism analysis (SSLPs), randomly amplified polymorphic DNAs (RAPDs), DNA amplification fingerprinting (DAF), sequence characterized amplified regions (SCARs), arbitrary primed polymerase chain reaction (AP-PCR), and single nucleotide polymorphisms (SNPs).

41. The method of claim 39 further comprising the step of performing at least one backcross of said progeny with said crossed strain.

42. A method for identifying a mutant gene conferring herbicide resistance comprising:
(a) crossing an herbicide resistant weed with an herbicide susceptible weed, thereby creating a crossed strain;
(b) screening progeny for herbicide resistance; and
(c) performing genetic analysis on said crossed strain producing herbicide resistant progeny to identify an herbicide resistance gene.

43. The method of claim 42 wherein said genome of said herbicide resistant plant and said genome of said herbicide susceptible plant are compared by a technique selected from the group consisting of microarray analysis, genotyping of repetitive sequences using microsatellite markers to identify linked genomic segments that are associated with a particular trait, single nucleotide polymorphic (SNP) analysis, restriction fragment length polymorphism (RFLP) analysis, amplified fragment length polymorphism (AFLP) analysis, simple sequence length polymorphism analysis (SSLPs), randomly amplified polymorphic DNAs (RAPDs), DNA amplification fingerprinting (DAF), sequence characterized amplified regions (SCARs), arbitrary primed polymerase chain reaction (AP-PCR), and single nucleotide polymorphisms (SNPs).

44. The method of claim 42 further comprising the step of performing at least one backcross of said progeny with said crossed strain.

45. A method for identifying a mutant gene conferring herbicide resistance comprising
(a) mutagenizing an herbicide susceptible weed, thereby creating mutant parental weeds;
(b) testing progeny of said mutant parental weeds for resistance to said herbicide; and
(c) comparing the genome of a naturally occurring herbicide resistant plant to the genome of an herbicide susceptible plant;
(d) determining genetic differences between said herbicide resistant plant to the herbicide susceptible plant; and
(e) sequencing a region of DNA comprising said genetic difference.

46. The method of claim 45 wherein said mutagenizing is accomplished by introducing into said herbicide resistant weed a dominant negative allele of a mismatch repair gene.

47. The method of claim 45 wherein said dominant negative allele of a mismatch gene is a dominant negative allele of a gene encoding a mismatch repair protein selected from the group consisting of PMS2, PMS1, MLH1, MSH2, MSH3, MSH6-1, MSH7, MSH6, PMSR2, PMSR3, and PMSL9.

48. The method of claim 47 wherein said dominant negative allele is a PMS2 truncation mutant.

49. The method of claim 48 wherein said truncation mutant encodes PMS2-134.

50. The method of claim 45 wherein said mutagenizing is accomplished by introducing into said herbicide resistant weed a chemical inhibitor of mismatch repair selected from the group consisting of an anthracene, an ATPase inhibitor, a nuclease inhibitor, a polymerase inhibitor and an antisense oligonucleotide that specifically hybridizes to a nucleotide encoding a mismatch repair protein dominant negative allele of a mismatch repair gene.

51. The method of claim 50 wherein said chemical inhibitor is an anthracene having the formula: 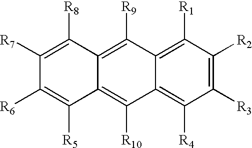
wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein said heteroalkyl, heteroaryl, and substituted heteroaryl contain at least one heteroatom that is oxygen, sulfur, a metal atom, phosphorus, silicon or nitrogen; and wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein said amino groups are optionally substituted with an acyl group, or 1 to 3 aryl or lower alkyl groups.

52. The method of claim 51 wherein R₅ and R₆ are hydrogen.

53. The method of claim 51 wherein R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

54. The method of claim 51 wherein said chemical inhibitor of mismatch repair is selected from the group consisting of 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

55. A method for identifying a mutant gene conferring herbicide resistance comprising
(a) mutagenizing an herbicide resistant weed, thereby creating mutant parental weeds;
(b) testing progeny of said mutant parental weeds for susceptibility to said herbicide; and
(c) comparing the genome of a naturally occurring herbicide resistant plant to the genome of an herbicide susceptible plant;
(d) determining genetic differences between said herbicide resistant plant to the herbicide susceptible plant; and
(e) sequencing a region of DNA comprising said genetic difference.

56. The method of claim 55 wherein said mutagenizing is accomplished by introducing into said herbicide resistant weed a dominant negative allele of a mismatch repair gene.

57. The method of claim 55 wherein said dominant negative allele of a mismatch gene is a dominant negative allele of a gene encoding a mismatch repair protein selected from the group consisting of PMS2, PMS1, MLH1, MSH2, MSH3, MSH6-1, MSH7, MSH6, PMSR2, PMSR3, and PMSL9.

58. The method of claim 57 wherein said dominant negative allele is a PMS2 truncation mutant.

59. The method of claim 58 wherein said truncation mutant encodes PMS2-134.

60. The method of claim 55 wherein said mutagenizing is accomplished by introducing into said herbicide resistant weed a chemical inhibitor of mismatch repair selected from the group consisting of an anthracene, an ATPase inhibitor, a nuclease inhibitor, a polymerase inhibitor and an antisense oligonucleotide that specifically hybridizes to a nucleotide encoding a mismatch repair protein dominant negative allele of a mismatch repair gene.

61. The method of claim 60 wherein said chemical inhibitor is an anthracene having the formula: 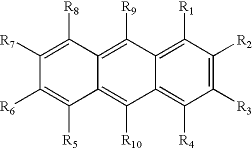
wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein said heteroalkyl, heteroaryl, and substituted heteroaryl contain at least one heteroatom that is oxygen, sulfur, a metal atom, phosphorus, silicon or nitrogen; and wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein said amino groups are optionally substituted with an acyl group, or 1 to 3 aryl or lower alkyl groups.

62. The method of claim 61 wherein R₅ and R₆ are hydrogen.

63. The method of claim 61 wherein R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

64. The method of claim 61 wherein said chemical inhibitor of mismatch repair is selected from the group consisting of 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

65. A polymorphic DNA marker for identifying herbicide resistant and herbicide susceptible weeds comprising a polynucleotide sequence encoding a polypeptide comprising SEQ ID NO: 17.

66. The polymorphic DNA marker of claim 65 wherein said polynucleotide comprises the sequence of SEQ ID NO: 16.

67. A kit for the identification of herbicide resistant and herbicide susceptible weeds comprising, in one or more containers, an oligonucleotide primer comprising the sequence of SEQ ID NO: 18, and a second oligonucleotide primer comprising the sequence of SEQ ID NO: 19.

68. The kit of claim 67 further comprising at least one other component selected from the group consisting of a DNA polymerase, deoxynucleotide triphosphates, genomic DNA from an herbicide susceptible plant, genomic DNA from an herbicide resistant plant, and DNA polymerase buffer.

69. A method for generating genetically stable glyphosate susceptible weeds derived from glyphosate resistant parental weeds comprising:
(a) contacting said glyphosate susceptible weed with an inhibitor of mismatch repair, thereby forming a hypermutable parental weed;
(b) testing progeny of said hypermutable parental weed that are glyphosate susceptible;
(c) selecting hypermutable parental strains producing glyphosate susceptible progeny;
(d) removing said inhibitor of mismatch repair from said hypermutable parental weed, thereby making said hypermutable parental weed genetically stable; and
(e) obtaining progeny from genetically stable parental weed.

70. The method of claim 69 wherein said inhibitor of mismatch repair is a dominant negative allele of a mismatch repair gene.

71. The method of claim 70 wherein said dominant negative allele of said mismatch repair gene is PMS2-134.

72. The method of claim 69 wherein said inhibitor of mismatch repair is a chemical inhibitor of mismatch repair.

73. The method of claim 72 wherein said chemical inhibitor of mismatch repair is selected from the group consisting of an anthracene, an ATPase inhibitor, a nuclease inhibitor, a polymerase inhibitor and an antisense oligonucleotide that specifically hybridizes to a nucleotide encoding a mismatch repair protein.

74. The method of claim 73 wherein said chemical inhibitor is an anthracene having the formula: 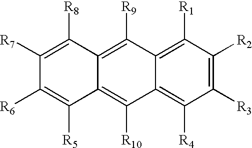
wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein said heteroalkyl, heteroaryl, and substituted heteroaryl contain at least one heteroatom that is oxygen, sulfur, a metal atom, phosphorus, silicon or nitrogen; and wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein said amino groups are optionally substituted with an acyl group, or 1 to 3 aryl or lower alkyl groups.

75. The method of claim 74 wherein R₅ and R₆ are hydrogen.

76. The method of claim 74 wherein R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl.

77. The method of claim 74 wherein said chemical inhibitor of mismatch repair is selected from the group consisting of 1,2-dimethylanthracene, 9,10-dimethylanthracene, 7,8-dimethylanthracene, 9,10-diphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

78. An oligonucleotide primer that anneals under PCR conditions to a polymorphic marker in genomic DNA or cDNA of a plant, wherein the nucleotide sequence of said polymorphic marker is selected from the group consisting of SEQ ID NO: 16, SEQ ID NO: 60, SEQ ID NO: 61, SEQ ID NO: 126, SEQ ID NO: 127, SEQ ID NO: 128, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, and SEQ ID NO: 111, and wherein said oligonucleotide primer is at least 15 nucleotides in length, and comprising at least 85% identity to a region of said polymorphic marker.

79. The oligonucleotide primer of claim 78 wherein said primer comprises a nucleotide sequence selected from the group consisting of SEQ ID NO: 112, SEQ ID NO: 113 SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, and SEQ ID NO: 125.

80. A kit for amplifying a polymorphic marker from a plant comprising in one or more containers, at least one oligonucleotide primer of claim 78 or 79.

81. A method for screening for herbicide resistant and herbicide susceptible plants comprising amplifying a polymorphic marker in a PCR-based assay using DNA from said plant, wherein said PCR comprises at least one primer comprising a sequence of SEQ ID NO: 18 or SEQ ID NO: 19.

82. The method of claim 81 wherein said plant is Conyza canadensis.

83. The method of claim 81 wherein said PCR-based assay further comprises at least one other component selected from the group consisting of a DNA polymerase, dNTPs, a primer comprising the sequence of SEQ ID NO: 20 and a primer comprising the sequence of SEQ ID NO: 21.

84. A method of identifying a therapeutic compound to increase a resistance to herbicides in a plant comprising:
(a) introducing a gene conferring herbicide susceptibility into a plant;
(b) isolating purified protein from said plant;
(c) contacting said protein with a panel of candidate compounds;
(d) selecting compounds that bind to said protein; and
(e) screening for the ability of a selected compound to interfere with herbicide susceptibility;
thereby identifying a therapeutic compound.


SEQUENCE LISTING (129 sequences)

SEQ ID NO: 1 - DNA, 16 bp, Artificial Sequence
  Oligonucleotide primer
gacgatgagtcctgag

SEQ ID NO: 2 - DNA, 14 bp, Artificial Sequence
  Oligonucleotide primer
tactcaggactcat

SEQ ID NO: 3 - DNA, 17 bp, Artificial Sequence
  Oligonucleotide primer
ctcgtagactgcgtacc

SEQ ID NO: 4 - DNA, 18 bp, Artificial Sequence
  Oligonucleotide primer
aattggtacgcagtctac

SEQ ID NO: 5 - DNA, 22 bp, Artificial Sequence
  Oligonucleotide primer
ctcgtagactgcgtaccaattc

SEQ ID NO: 6 - DNA, 20 bp, Artificial Sequence
  Oligonucleotide primer
gacgatgagtcctgagtacc

SEQ ID NO: 7 - DNA, 24 bp, Artificial Sequence
  Oligonucleotide primer
ctcgtagactgcgtaccaattctg

SEQ ID NO: 8 - DNA, 22 bp, Artificial Sequence
  Oligonucleotide primer
gacgatgagtcctgagtaccac

SEQ ID NO: 9 - DNA, 16 bp, Artificial Sequence
  Degenerate oligonucleotide primer
ngtgnagnancanaga

SEQ ID NO: 10 - DNA, 18 bp, Artificial Sequence
  Oligonucleotide primer
cacatctttagcatcggc

SEQ ID NO: 11 - DNA, 18 bp, Artificial Sequence
  Oligonucleotide primer
aaagtggctgaatcgtgg

SEQ ID NO: 12 - DNA, 18 bp, Artificial Sequence
  Oligonucleotide primer
gatttgaatggtggtgcc

SEQ ID NO: 13 - DNA, 18 bp, Artificial Sequence
  Oligonucleotide primer
ggcaccaccattcaaatc

SEQ ID NO: 14 - DNA, 18 bp, Artificial Sequence
  Oligonucleotide primer
ataccacgattcagccac

SEQ ID NO: 15 - DNA, 18 bp, Artificial Sequence
  Oligonucleotide primer
gccgatgctaaagatgtg

SEQ ID NO: 16 - DNA, 919 bp, Conyza sp.
ataccacgattcagccaccatacaaccgccgcctgatcaattgaaggctggggggggattcgattgtgggtggggcagttccaaatctcactattgatgctcaattaatctatttaggggttttgcaaaccgaaaccctaatataaaaaccttaatttgttgcttgtcgctgtccaaccattgtccacccttctctgaaccacagtttggaaatttaattgatggggagggagatttttcgaacctggatggcaattgattactcacttagccacaagttgatgaagtggatcgcatattggccaaaaaggtcattttcatgtgccgtcatgtgaagatagtgaacaaattgatgatatatttaccatctttaaggatgaaatggaaatgtttaatgatactgataatccactctctaaagatcaagtggccgaattgaatagaattttagttgtacattgggctaaaatcactaattttgagggctcgaattcacagcaacgacctttacttccggatgaaattgaaaagcattttggtgttcaaccttgtgacaatattaacacatctttagcatcggccaccattgaaaaagtggctgaatcgtggtataagaatgttgtattgcaggttgatgttgagagggatttggatgatttgaatggtggtgccagaattctactgctgagtcatctttgcatgatttccatgcaaaaggtggtgctactcatgtttcccctatgcttgatcctcctaagtttcctcctggtactacttattttaagccagctacagaccatgccaatgacattcttgatgtttcagatgatgttcctaagcatgatgtttctccgaatgatcttggtggacatccagatagccccaaaactcagcagcctcacttctgtgcttctccaca

SEQ ID NO: 17 - protein, 179 aa, Conyza sp.
MEMFNDTDNPLSKDQVAELNRILVVHWAKITNFEGSNSQQRPLLPDEIEKHFGVQPCDNINTSLASATIEKVAESWYKNVVLQVDVERDLDDLNGGARILLLSHLCMISMQKVVLLMFPLCLILLSFLLVLLILSQLQTMPMTFLMFQMMFLSMMFLRMILVDIQIAPKLSSLTSVLLH

SEQ ID NO: 18 - DNA, 20 bp, Artificial Sequence
  Oligonucleotide primer
ttgtcgctgtccaaccattg

SEQ ID NO: 19 - DNA, 20 bp, Artificial Sequence
  Oligonucleotide primer
ttggcatggtctgtagctgg

SEQ ID NO: 20 - DNA, 19 bp, Artificial Sequence
  Oligonucleotide primer
ccatcgtatcatcatgtgc

SEQ ID NO: 21 - DNA, 21 bp, Artificial Sequence
  Oligonucleotide primer
tgcaatatgttaaagtagagc

SEQ ID NO: 22 - DNA, 3218 bp, Saccharomyces cerevisiae
aaataggaatgtgataccttctattgcatgcaaagatagtgtaggaggcgctgctattgccaaagacttttgagaccgcttgctgtttcattatagttgaggagttctcgaagacgagaaattagcagttttcggtgtttagtaatcgcgctagcatgctaggacaatttaactgcaaaattttgatacgatagtgatagtaaatggaaggtaaaaataacatagacctatcaataagcaatgtctctcagaataaaagcacttgatgcatcagtggttaacaaaattgctgcaggtgagatcataatatcccccgtaaatgctctcaaagaaatgatggagaattccatcgatgcgaatgctacaatgattgatattctagtcaaggaaggaggaattaaggtacttcaaataacagataacggatctggaattaataaagcagacctgccaatcttatgtgagcgattcacgacgtccaaattacaaaaattcgaagatttgagtcagattcaaacgtatggattccgaggagaagctttagccagtatctcacatgtggcaagagtcacagtaacgacaaaagttaaagaagacagatgtgcatggagagtttcatatgcagaaggtaagatgttggaaagccccaaacctgttgctggaaaagacggtaccacgatcctagttgaagacctttttttcaatattccttctagattaagggccttgaggtcccataatgatgaatactctaaaatattagatgttgtcgggcgatacgccattcattccaaggacattggcttttcttgtaaaaagttcggagactctaattattctttatcagttaaaccttcatatacagtccaggataggattaggactgtgttcaataaatctgtggcttcgaatttaattacttttcatatcagcaaagtagaagatttaaacctggaaagcgttgatggaaaggtgtgtaatttgaatttcatatccaaaaagtccatttcattaatttttttcattaataatagactagtgacatgtgatcttctaagaagagctttgaacagcgtttactccaattatctgccaaagggcttcagaccttttatttatttgggaattgttatagatccggcggctgttgatgttaacgttcacccgacaaagagagaggttcgtttcctgagccaagatgagatcatagagaaaatcgccaatcaattgcacgccgaattatctgccattgatacttcacgtactttcaaggcttcttcaatttcaacaaacaagccagagtcattgataccatttaatgacaccatagaaagtgataggaataggaagagtctccgacaagcccaagtggtagagaattcatatacgacagccaatagtcaactaaggaaagcgaaaagacaagagaataaactagtcagaatagatgcttcacaagctaaaattacgtcatttttatcctcaagtcaacagttcaactttgaaggatcgtctacaaagcgacaactgagtgaacccaaggtaacaaatgtaagccactcccaagaggcagaaaagctgacactaaatgaaagcgaacaaccgcgtgatgccaatacaatcaatgataatgacttgaaggatcaacctaagaagaaacaaaagttgggggattataaagttccaagcattgccgatgacgaaaagaatgcactcccgatttcaaaagacgggtatattagagtacctaaggagcgagttaatgttaatcttacgagtatcaagaaattgcgtgaaaaagtagatgattcgatacatcgagaactaacagacatttttgcaaatttgaattacgttggggttgtagatgaggaaagaagattagccgctattcagcatgacttaaagctttttttaatagattacggatctgtgtgctatgagctattctatcagattggtttgacagacttcgcaaactttggtaagataaacctacagagtacaaatgtgtcagatgatatagttttgtataatctcctatcagaatttgacgagttaaatgacgatgcttccaaagaaaaaataattagtaaaatatgggacatgagcagtatgctaaatgagtactattccatagaattggtgaatgatggtctagataatgacttaaagtctgtgaagctaaaatctctaccactacttttaaaaggctacattccatctctggtcaagttaccattttttatatatcgcctgggtaaagaagttgattgggaggatgaacaagagtgtctagatggtattttaagagagattgcattactctatatacctgatatggttccgaaagtcgatacactcgatgcatcgttgtcagaagacgaaaaagcccagtttataaatagaaaggaacacatatcctcattactagaacacgttctcttcccttgtatcaaacgaaggttcctggcccctagacacattctcaaggatgtcgtggaaatagccaaccttccagatctatacaaagtttttgagaggtgttaactttaaaacgttttggctgtaataccaaagtttttgtttatttcctgagtgtgattgtgtttcatttgaaagtgtatgccctttcctttaacgattcatccgcgagatttcaaaggatatgaaatatggttgcagttaggaaagtatgtcagaaatgtatattcggattgaaactcttctaatagttctgaagtcacttggttccgtattgttttcgtcctcttcctcaagcaacgattcttgtctaagcttattcaacggtaccaaagacccgagtccttttatgagagaaaacatttcatcatttttcaactcaattatcttaatatcattttgtagtattttgaaaacaggatggtaaaacgaatcacctgaatctagaagctgtaccttgtcccataaaagttttaatttactgagcctttcggtcaagtaaactagtttatctagttttgaaccgaatattgtgggcagatttgcagtaagttcagttagatctactaaaagttgtttgacagcagccgattccacaaaaatttggtaaaaggagatgaaagagacctcgcgcgtaatggtttgcatcaccatcggatgtctgttgaaaaactcactttttgcatggaagttattaacaataagactaatgattaccttagaataatgtataa

SEQ ID NO: 23 - protein, 769 aa, Saccharomyces cerevisiae
MSLRIKALDASVVNKIAAGEIIISPVNALKEMMENSIDANATMIDILVKEGGIKVLQITDNGSGINKADLPILCERFTTSKLQKFEDLSQIQTYGFRGEALASISHVARVTVTTKVKEDRCAWRVSYAEGKMLESPKPVAGKDGTTILVEDLFFNIPSRLRALRSHNDEYSKILDVVGRYAIHSKDIGFSCKKFGDSNYSLSVKPSYTVQDRIRTVFNKSVASNLITFHISKVEDLNLESVDGKVCNLNFISKKSISLIFFINNRLVTCDLLRRALNSVYSNYLPKGFRPFIYLGIVIDPAAVDVNVHPTKREVRFLSQDEIIEKIANQLHAELSAIDTSRTFKASSISTNKPESLIPFNDTIESDRNRKSLRQAQVVENSYTTANSQLRKAKRQENKLVRIDASQAKITSFLSSSQQFNFEGSSTKRQLSEPKVTNVSHSQEAEKLTLNESEQPRDANTINDNDLKDQPKKKQKLGDYKVPSIADDEKNALPISKDGYIRVPKERVNVNLTSIKKLREKVDDSIHRELTDIFANLNYVGVVDEERRLAAIQHDLKLFLIDYGSVCYELFYQIGLTDFANFGKINLQSTNVSDDIVLYNLLSEFDELNDDASKEKIISKIWDMSSMLNEYYSIELVNDGLDNDLKSVKLKSLPLLLKGYIPSLVKLPFFIYRLGKEVDWEDEQECLDGILREIALLYIPDMVPKVDTLDASLSEDEKAQFINRKEHISSLLEHVLFPCIKRRFLAPRHILKDVVEIANLPDLYKVFERC

SEQ ID NO: 24 - DNA, 3056 bp, Mus musculus
gaattccggtgaaggtcctgaagaatttccagattcctgagtatcattggaggagacagataacctgtcgtcaggtaacgatggtgtatatgcaacagaaatgggtgttcctggagacgcgtcttttcccgagagcggcaccgcaactctcccgcggtgactgtgactggaggagtcctgcatccatggagcaaaccgaaggcgtgagtacagaatgtgctaaggccatcaagcctattgatgggaagtcagtccatcaaatttgttctgggcaggtgatactcagtttaagcaccgctgtgaaggagttgatagaaaatagtgtagatgctggtgctactactattgatctaaggcttaaagactatggggtggacctcattgaagtttcagacaatggatgtggggtagaagaagaaaactttgaaggtctagctctgaaacatcacacatctaagattcaagagtttgccgacctcacgcaggttgaaactttcggctttcggggggaagctctgagctctctgtgtgcactaagtgatgtcactatatctacctgccacgggtctgcaagcgttgggactcgactggtgtttgaccataatgggaaaatcacccagaaaactccctacccccgacctaaaggaaccacagtcagtgtgcagcacttattttatacactacccgtgcgttacaaagagtttcagaggaacattaaaaaggagtattccaaaatggtgcaggtcttacaggcgtactgtatcatctcagcaggcgtccgtgtaagctgcactaatcagctcggacaggggaagcggcacgctgtggtgtgcacaagcggcacgtctggcatgaaggaaaatatcgggtctgtgtttggccagaagcagttgcaaagcctcattccttttgttcagctgccccctagtgacgctgtgtgtgaagagtacggcctgagcacttcaggacgccacaaaaccttttctacgtttcgggcttcatttcacagtgcacgcacggcgccgggaggagtgcaacagacaggcagtttttcttcatcaatcagaggccctgtgacccagcaaaggtctctaagcttgtcaatgaggttttatcacatgtataaccggcatcagtacccatttgtcgtccttaacgtttccgttgactcagaatgtgtggatattaatgtaactccagataaaaggcaaattctactacaagaagagaagctattgctggccgttttaaagacctccttgataggaatgtttgacagtgatgcaaacaagcttaatgtcaaccagcagccactgctagatgttgaaggtaacttagtaaagctgcatactgcagaactagaaaagcctgtgccaggaaagcaagataactctccttcactgaagagcacagcagacgagaaaagggtagcatccatctccaggctgagagaggccttttctcttcatcctactaaagagatcaagtctaggggtccagagactgctgaactgacacggagttttccaagtgagaaaaggggcgtgttatcctcttatccttcagacgtcatctcttacagaggcctccgtggctcgcaggacaaattggtgagtcccacggacagccctggtgactgtatggacagagagaaaatagaaaaagactcagggctcagcagcacctcagctggctctgaggaagagttcagcaccccagaagtggccagtagctttagcagtgactataacgtgagctccctagaagacagaccttctcaggaaaccataaactgtggtgacctggactgccgtcctccaggtacaggacagtccttgaagccagaagaccatggatatcaatgcaaagctctacctctagctcgtctgtcacccacaaatgccaagcgcttcaagacagaggaaagaccctcaaatgtcaacatttctcaaagattgcctggtcctcagagcacctcagcagctgaggtcgatgtagccataaaaatgaataagagaatcgtgctcctcgagttctctctgagttctctagctaagcgaatgaagcagttacagcacctaaaggcgcagaacaaacatgaactgagttacagaaaatttagggccaagatttgccctggagaaaaccaagcagcagaagatgaactcagaaaagagattagtaaatcgatgtttgcagagatggagatcttgggtcagtttaacctgggatttatagtaaccaaactgaaagaggacctcttcctggtggaccagcatgctgcggatgagaagtacaactttgagatgctgcagcagcacacggtgctccaggcgcagaggctcatcacaccccagactctgaacttaactgctgtcaatgaagctgtactgatagaaaatctggaaatattcagaaagaatggctttgactttgtcattgatgaggatgctccagtcactgaaagggctaaattgatttccttaccaactagtaaaaactggacctttggaccccaagatatagatgaactgatctttatgttaagtgacagccctggggtcatgtgccggccctcacgagtcagacagatgtttgcttccagagcctgtcggaagtcagtgatgattggaacggcgctcaatgcgagcgagatgaagaagctcatcacccacatgggtgagatggaccacccctggaactgcccccacggcaggccaaccatgaggcacgttgccaatctggatgtcatctctcagaactgacacaccccttgtagcatagagtttattacagattgttcggtttgcaaagagaaggttttaagtaatctgattatcgttgtacaaaaattagcatgctgctttaatgtactggatccatttaaaagcagtgttaaggcaggcatgatggagtgttcctctagctcagctacttgggtgatccggtgggagctcatgtgagcccaggactttgagaccactccgagccacattcatgagactcaattcaaggacaaaaaaaaaaagatatttttgaagccttttaaaaaaaaa

SEQ ID NO: 25 - protein, 859 aa, Mus musculus
MEQTEGVSTECAKAIKPIDGKSVHQICSGQVILSLSTAVKELIENSVDAGATTIDLRLKDYGVDLIEVSDNGCGVEEENFEGLALKHHTSKIQEFADLTQVETFGFRGEALSSLCALSDVTISTCHGSASVGTRLVFDHNGKITQKTPYPRPKGTTVSVQHLFYTLPVRYKEFQRNIKKEYSKMVQVLQAYCIISAGVRVSCTNQLGQGKRHAVVCTSGTSGMKENIGSVFGQKQLQSLIPFVQLPPSDAVCEEYGLSTSGRHKTFSTFRASFHSARTAPGGVQQTGSFSSSIRGPVTQQRSLSLSMRFYHMYNRHQYPFVVLNVSVDSECVDINVTPDKRQILLQEEKLLLAVLKTSLIGMFDSDANKLNVNQQPLLDVEGNLVKLHTAELEKPVPGKQDNSPSLKSTADEKRVASISRLREAFSLHPTKEIKSRGPETAELTRSFPSEKRGVLSSYPSDVISYRGLRGSQDKLVSPTDSPGDCMDREKIEKDSGLSSTSAGSEEEFSTPEVASSFSSDYNVSSLEDRPSQETINCGDLDCRPPGTGQSLKPEDHGYQCKALPLARLSPTNAKRFKTEERPSNVNISQRLPGPQSTSAAEVDVAIKMNKRIVLLEFSLSSLAKRMKQLQHLKAQNKHELSYRKFRAKICPGENQAAEDELRKEISKSMFAEMEILGQFNLGFIVTKLKEDLFLVDQHAADEKYNFEMLQQHTVLQAQRLITPQTLNLTAVNEAVLIENLEIFRKNGFDFVIDEDAPVTERAKLISLPTSKNWTFGPQDIDELIFMLSDSPGVMCRPSRVRQMFASRACRKSVMIGTALNASEMKKLITHMGEMDHPWNCPHGRPTMRHVANLDVISQN

SEQ ID NO: 26 - DNA, 2771 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttcggatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctgaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactgaccgtagtcactgtatggaataattggttttatcgcagatttttatgttttgaaagacagagtcttcactaaccttttttgttttaaaatgaaacctgctacttaaaaaaaatacacatcacacccatttaaaagtgatcttgagaaccttttcaaacc

SEQ ID NO: 27 - protein, 932 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQKPSHLGQGTTVTALRLFKNLPVRKQFYSTAKKCKDEIKKIQDLLMSFGILKPDLRIVFVHNKAVIWQKSRVSDHKMALMSVLGTAVMNNMESFQYHSEESQIYLSGFLPKCDADHSFTSLSTPERSFIFINSRPVHQKDILKLIRHHYNLKCLKESTRLYPVFFLKIDVPTADVDVNLTPDKSQVLLQNKESVLIALENLMTTCYGPLPSTNSYENNKTDVSAADIVLSKTAETDVLFNKVESSGKNYSNVDTSVIPFQNDMHNDESGKNTDDCLNHQISIGDFGYGHCSSEISNIDKNTKNAFQDISMSNVSWENSQTEYSKTCFISSVKHTQSENGNKDHIDESGENEEEAGLENSSEISADEWSRGNILKNSVGENIEPVKILVPEKSLPCKVSNNNYPIPEQMNLNEDSCNKKSNVIDNKSGKVTAYDLLSNRVIKKPMSASALFVQDHRPQFLIENPKTSLEDATLQIEELWKTLSEEEKLKYEEKATKDLERYNSQMKRAIEQESQMSLKDGRKKIKPTSAWNLAQKHKLKTSLSNQPKLDELLQSQIEKRRSQNIKMVQIPFSMKNLKINFKKQNKVDLEEKDEPCLIHNLRFPDAWLMTSKTEVMLLNPYRVEEALLFKRLLENHKLPAEPLEKPIMLTESLFNGSHYLDVLYKMTADDQRYSGSTYLSDPRLTANGFKIKLIPGVSITENYLEIEGMANCLPFYGVADLKEILNAILNRNAKEVYECRPRKVISYLEGEAVRLSRQLPMYLSKEDIQDIIYRMKHQFGNEIKECVHGRPFFHHLTYLPETT

SEQ ID NO: 28 - DNA, 3063 bp, Homo sapiens
ggcacgagtggctgcttgcggctagtggatggtaattgcctgcctcgcgctagcagcaagctgctctgttaaaagcgaaaatgaaacaattgcctgcggcaacagttcgactcctttcaagttctcagatcatcacttcggtggtcagtgttgtaaaagagcttattgaaaactccttggatgctggtgccacaagcgtagatgttaaactggagaactatggatttgataaaattgaggtgcgagataacggggagggtatcaaggctgttgatgcacctgtaatggcaatgaagtactacacctcaaaaataaatagtcatgaagatcttgaaaatttgacaacttacggttttcgtggagaagccttggggtcaatttgttgtatagctgaggttttaattacaacaagaacggctgctgataattttagcacccagtatgttttagatggcagtggccacatactttctcagaaaccttcacatcttggtcaaggtacaactgtaactgctttaagattatttaagaatctacctgtaagaaagcagttttactcaactgcaaaaaaatgtaaagatgaaataaaaaagatccaagatctcctcatgagctttggtatccttaaacctgacttaaggattgtctttgtacataacaaggcagttatttggcagaaaagcagagtatcagatcacaagatggctctcatgtcagttctggggactgctgttatgaacaatatggaatcctttcagtaccactctgaagaatctcagatttatctcagtggatttcttccaaagtgtgatgcagaccactctttcactagtctttcaacaccagaaagaagtttcatcttcataaacagtcgaccagtacatcaaaaagatatcttaaagttaatccgacatcattacaatctgaaatgcctaaaggaatctactcgtttgtatcctgttttctttctgaaaatcgatgttcctacagctgatgttgatgtaaatttaacaccagataaaagccaagtattattacaaaataaggaatctgttttaattgctcttgaaaatctgatgacgacttgttatggaccattacctagtacaaattcttatgaaaataataaaacagatgtttccgcagctgacatcgttcttagtaaaacagcagaaacagatgtgctttttaataaagtggaatcatctggaaagaattattcaaatgttgatacttcagtcattccattccaaaatgatatgcataatgatgaatctggaaaaaacactgatgattgtttaaatcaccagataagtattggtgactttggttatggtcattgtagtagtgaaatttctaacattgataaaaacactaagaatgcatttcaggacatttcaatgagtaatgtatcatgggagaactctcagacggaatatagtaaaacttgttttataagttccgttaagcacacccagtcagaaaatggcaataaagaccatatagatgagagtggggaaaatgaggaagaagcaggtcttgaaaactcttcggaaatttctgcagatgagtggagcaggggaaatatacttaaaaattcagtgggagagaatattgaacctgtgaaaattttagtgcctgaaaaaagtttaccatgtaaagtaagtaataataattatccaatccctgaacaaatgaatcttaatgaagattcatgtaacaaaaaatcaaatgtaatagataataaatctggaaaagttacagcttatgatttacttagcaatcgagtaatcaagaaacccatgtcagcaagtgctctttttgttcaagatcatcgtcctcagtttctcatagaaaatcctaagactagtttagaggatgcaacactacaaattgaagaactgtggaagacattgagtgaagaggaaaaactgaaatatgaagagaaggctactaaagacttggaacgatacaatagtcaaatgaagagagccattgaacaggagtcacaaatgtcactaaaagatggcagaaaaaagataaaacccaccagcgcatggaatttggcccagaagcacaagttaaaaacctcattatctaatcaaccaaaacttgatgaactccttcagtcccaaattgaaaaaagaaggagtcaaaatattaaaatggtacagatccccttttctatgaaaaacttaaaaataaattttaagaaacaaaacaaagttgacttagaagagaaggatgaaccttgcttgatccacaatctcaggtttcctgatgcatggctaatgacatccaaaacagaggtaatgttattaaatccatatagagtagaagaagccctgctatttaaaagacttcttgagaatcataaacttcctgcagagccactggaaaagccaattatgttaacagagagtctttttaatggatctcattatttagacgttttatataaaatgacagcagatgaccaaagatacagtggatcaacttacctgtctgatcctcgtcttacagcgaatggtttcaagataaaattgataccaggagtttcaattactgaaaattacttggaaatagaaggaatggctaattgtctcccattctatggagtagcagatttaaaagaaattcttaatgctatattaaacagaaatgcaaaggaagtttatgaatgtagacctcgcaaagtgataagttatttagagggagaagcagtgcgtctatccagacaattacccatgtacttatcaaaagaggacatccaagacattatctacagaatgaagcaccagtttggaaatgaaattaaagagtgtgttcatggtcgcccattttttcatcatttaacctatcttccagaaactacatgattaaatatgtttaagaagattagttaccattgaaattggttctgtcataaaacagcatgagtctggttttaaattatctttgtattatgtgtcacatggttattttttaaatgaggattcactgacttgtttttatattgaaaaaagttccacgtattgtagaaaacgtaaataaactaataac

SEQ ID NO: 29 - protein, 932 aa, Homo sapiens; identical to SEQ ID NO: 27

SEQ ID NO: 30 - DNA, 3145 bp, Homo sapiens
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 31 - protein, 934 aa, Homo sapiens [sequence omitted]

SEQ ID NO: 32 - DNA, 2484 bp, Homo sapiens
cttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacata

SEQ ID NO: 33 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 34 - DNA, 426 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaacttga

SEQ ID NO: 35 - protein, 133 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQK

SEQ ID NO: 36 - DNA, 4264 bp, Homo sapiens
atttcccgccagcaggagccgcgcggtagatgcggtgcttttaggagctccgtccgacagaacggttgggccttgccggctgtcggtatgtcgcgacagagcaccctgtacagcttcttccccaagtctccggcgctgagtgatgccaacaaggcctcggccagggcctcacgcgaaggcggccgtgccgccgctgcccccggggcctctccttccccaggcggggatgcggcctggagcgaggctgggcctgggcccaggcccttggcgcgatccgcgtcaccgcccaaggcgaagaacctcaacggagggctgcggagatcggtagcgcctgctgcccccaccagttgtgacttctcaccaggagatttggtttgggccaagatggagggttacccctggtggccttgtctggtttacaaccacccctttgatggaacattcatccgcgagaaagggaaatcagtccgtgttcatgtacagttttttgatgacagcccaacaaggggctgggttagcaaaaggcttttaaagccatatacaggttcaaaatcaaaggaagcccagaagggaggtcatttttacagtgcaaagcctgaaatactgagagcaatgcaacgtgcagatgaagccttaaataaagacaagattaagaggcttgaattggcagtttgtgatgagccctcagagccagaagaggaagaagagatggaggtaggcacaacttacgtaacagataagagtgaagaagataatgaaattgagagtgaagaggaagtacagcctaagacacaaggatctaggcgaagtagccgccaaataaaaaaacgaagggtcatatcagattctgagagtgacattggtggctctgatgtggaatttaagccagacactaaggaggaaggaagcagtgatgaaataagcagtggagtgggggatagtgagagtgaaggcctgaacagccctgtcaaagttgctcgaaagcggaagagaatggtgactggaaatggctctcttaaaaggaaaagctctaggaaggaaacgccctcagccaccaaacaagcaactagcatttcatcagaaaccaagaatactttgagagctttctctgcccctcaaaattctgaatcccaagcccacgttagtggaggtggtgatgacagtagtcgccctactgtttggtatcatgaaactttagaatggcttaaggaggaaaagagaagagatgagcacaggaggaggcctgatcaccccgattttgatgcatctacactctatgtgcctgaggatttcctcaattcttgtactcctgggatgaggaagtggtggcagattaagtctcagaactttgatcttgtcatctgttacaaggtggggaaattttatgagctgtaccacatggatgctcttattggagtcagtgaactggggctggtattcatgaaaggcaactgggcccattctggctttcctgaaattgcatttggccgttattcagattccctggtgcagaagggctataaagtagcacgagtggaacagactgagactccagaaatgatggaggcacgatgtagaaagatggcacatatatccaagtatgatagagtggtgaggagggagatctgtaggatcattaccaagggtacacagacttacagtgtgctggaaggtgatccctctgagaactacagtaagtatcttcttagcctcaaagaaaaagaggaagattcttctggccatactcgtgcatatggtgtgtgctttgttgatacttcactgggaaagtttttcataggtcagttttcagatgatcgccattgttcgagatttaggactctagtggcacactatcccccagtacaagttttatttgaaaaaggaaatctctcaaaggaaactaaaacaattctaaagagttcattgtcctgttctcttcaggaaggtctgatacccggctcccagttttgggatgcatccaaaactttgagaactctccttgaggaagaatattttagggaaaagctaagtgatggcattggggtgatgttaccccaggtgcttaaaggtatgacttcagagtctgattccattgggttgacaccaggagagaaaagtgaattggccctctctgctctaggtggttgtgtcttctacctcaaaaaatgccttattgatcaggagcttttatcaatggctaattttgaagaatatattcccttggattctgacacagtcagcactacaagatctggtgctatcttcaccaaagcctatcaacgaatggtgctagatgcagtgacattaaacaacttggagatttttctgaatggaacaaatggttctactgaaggaaccctactagagagggttgatacttgccatactccttttggtaagcggctcctaaagcaatggctttgtgccccactctgtaaccattatgctattaatgatcgtctagatgccatagaagacctcatggttgtgcctgacaaaatctccgaagttgtagagcttctaaagaagcttccagatcttgagaggctactcagtaaaattcataatgttgggtctcccctgaagagtcagaaccacccagacagcagggctataatgtatgaagaaactacatacagcaagaagaagattattgattttctttctgctctggaaggattcaaagtaatgtgtaaaattatagggatcatggaagaagttgctgatggttttaagtctaaaatccttaagcaggtcatctctctgcagacaaaaaatcctgaaggtcgttttcctgatttgactgtagaattgaaccgatgggatacagcctttgaccatgaaaaggctcgaaagactggacttattactcccaaagcaggctttgactctgattatgaccaagctcttgctgacataagagaaaatgaacagagcctcctggaatacctagagaaacagcgcaacagaattggctgtaggaccatagtctattgggggattggtaggaaccgttaccagctggaaattcctgagaatttcaccactcgcaatttgccagaagaatacgagttgaaatctaccaagaagggctgtaaacgatactggaccaaaactattgaaaagaagttggctaatctcataaatgctgaagaacggagggatgtatcattgaaggactgcatgcggcgactgttctataactttgataaaaattacaaggactggcagtctgctgtagagtgtatcgcagtgttggatgttttactgtgcctggctaactatagtcgagggggtgatggtcctatgtgtcgcccagtaattctgttgccggaagataccccccccttcttagagcttaaaggatcacgccatccttgcattacgaagactttttttggagatgattttattcctaatgacattctaataggctgtgaggaagaggagcaggaaaatggcaaagcctattgtgtgcttgttactggaccaaatatggggggcaagtctacgcttatgagacaggctggcttattagctgtaatggcccagatgggttgttacgtccctgctgaagtgtgcaggctcacaccaattgatagagtgtttactagacttggtgcctcagacagaataatgtcaggtgaaagtacattttttgttgaattaagtgaaactgccagcatactcatgcatgcaacagcacattctctggtgcttgtggatgaattaggaagaggtactgcaacatttgatgggacggcaatagcaaatgcagttgttaaagaacttgctgagactataaaatgtcgtacattattttcaactcactaccattcattagtagaagattattctcaaaatgttgctgtgcgcctaggacatatggcatgcatggtagaaaatgaatgtgaagaccccagccaggagactattacgttcctctataaattcattaagggagcttgtcctaaaagctatggctttaatgcagcaaggcttgctaatctcccagaggaagttattcaaaagggacatagaaaagcaagagaatttgagaagatgaatcagtcactacgattatttcgggaagtttgcctggctagtgaaaggtcaactgtagatgctgaagctgtccataaattgctgactttgattaaggaattatagactgactacattggaagctttgagttgacttctgaccaaaggtggtaaattcagacaacattatgatctaataaactttattttttaaaaatga

SEQ ID NO: 37 - protein, 1360 aa, Homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 38 - DNA, 1408 bp, Homo sapiens
ggcgctcctacctgcaagtggctagtgccaagtgctgggccgccgctcctgccgtgcatgttggggagccagtacatgcaggtgggctccacacggagaggggcgcagacccggtgacagggctttacctggtacatcggcatggcgcaaccaaagcaagagagggtggcgcgtgccagacaccaacggtcggaaaccgccagacaccaacggtcggaaaccgccaagacaccaacgctcggaaaccgccagacaccaacgctcggaaaccgccagacaccaaggctcggaatccacgccaggccacgacggagggcgactacctcccttctgaccctgctgctggcgttcggaaaaaacgcagtccggtgtgctctgattggtccaggctctttgacgtcacggactcgacctttgacagagccactaggcgaaaaggagagacgggaagtattttttccgccccgcccggaaagggtggagcacaacgtcgaaagcagccgttgggagcccaggaggcggggcgcctgtgggagccgtggagggaactttcccagtccccgaggcggatccggtgttgcatccttggagcgagctgagaactcgagtacagaacctgctaaggccatcaaacctattgatcggaagtcagtccatcagatttgctctgggccggtggtaccgagtctaaggccgaatgcggtgaaggagttagtagaaaacagtctggatgctggtgccactaatgttgatctaaagcttaaggactatggagtggatctcattgaagtttcaggcaatggatgtggggtagaagaagaaaacttcgaaggctttactctgaaacatcacacatgtaagattcaagagtttgccgacctaactcaggtggaaacttttggctttcggggggaagctctgagctcactttgtgcactgagtgatgtcaccatttctacctgccgtgtatcagcgaaggttgggactcgactggtgtttgatcactatgggaaaatcatccagaaaaccccctacccccgccccagagggatgacagtcagcgtgaagcagttattttctacgctacctgtgcaccataaagaatttcaaaggaatattaagaagaaacgtgcctgcttccccttcgccttctgccgtgattgtcagtttcctgaggcctccccagccatgcttcctgtacagcctgtagaactgactcctagaagtaccccaccccacccctgctccttggaggacaacgtgatcactgtattcagctctgtcaagaatggtccaggttcttctagatgatctgcacaaatggttcctctcctccttcctgatgtctgccattagcattggaataaagttcctgctgaaaatccaaaaaaaaaaaaaaaaaaaaaaa

SEQ ID NO: 39 - protein, 389 aa, Homo sapiens
MAQPKQERVARARHQRSETARHQRSETAKTPTLGNRQTPTLGNRQTPRLGIHARPRRRATTSLLTLLLAFGKNAVRCALIGPGSLTSRTRPLTEPLGEKERREVFFPPRPERVEHNVESSRWEPRRRGACGSRGGNFPSPRGGSGVASLERAENSSTEPAKAIKPIDRKSVHQICSGPVVPSLRPNAVKELVENSLDAGATNVDLKLKDYGVDLIEVSGNGCGVEEENFEGFTLKHHTCKIQEFADLTQVETFGFRGEALSSLCALSDVTISTCRVSAKVGTRLVFDHYGKIIQKTPYPRPRGMTVSVKQLFSTLPVHHKEFQRNIKKKRACFPFAFCRDCQFPEASPAMLPVQPVELTPRSTPPHPCSLEDNVITVFSSVKNGPGSSR

SEQ ID NO: 40 - DNA, 1785 bp, Homo sapiens
tttttagaaactgatgtttattttccatcaaccatttttccatgctgcttaagagaatatgcaagaacagcttaagaccagtcagtggttgctcctacccattcagtggcctgagcagtggggagctgcagaccagtcttccgtggcaggctgagcgctccagtcttcagtagggaattgctgaataggcacagagggcacctgtacaccttcagaccagtctgcaacctcaggctgagtagcagtgaactcaggagcgggagcagtccattcaccctgaaattcctccttggtcactgccttctcagcagcagcctgctcttctttttcaatctcttcaggatctctgtagaagtacagatcaggcatgacctcccatgggtgttcacgggaaatggtgccacgcatgcgcagaacttcccgagccagcatccaccacattaaacccactgagtgagctcccttgttgttgcatgggatggcaatgtccacatagcgcagaggagaatctgtgttacacagcgcaatggtaggtaggttaacataagatgcctccgtgagaggcgaaggggcggcgggacccgggcctggcccgtatgtgtccttggcggcctagactaggccgtcgctgtatggtgagccccagggaggcggatctgggcccccagaaggacacccgcctggatttgccccgtagcccggcccgggcccctcgggagcagaacagccttggtgaggtggacaggaggggacctcgcgagcagacgcgcgcgccagcgacagcagccccgccccggcctctcgggagccggggggcagaggctgcggagccccaggagggtctatcagccacagtctctgcatgtttccaagagcaacaggaaatgaacacattgcaggggccagtgtcattcaaagatgtggctgtggatttcacccaggaggagtggcggcaactggaccctgatgagaagatagcatacggggatgtgatgttggagaactacagccatctagtttctgtggggtatgattatcaccaagccaaacatcatcatggagtggaggtgaaggaagtggagcagggagaggagccgtggataatggaaggtgaatttccatgtcaacatagtccagaacctgctaaggccatcaaacctattgatcggaagtcagtccatcagatttgctctgggccagtggtactgagtctaagcactgcagtgaaggagttagtagaaaacagtctggatgctggtgccactaatattgatctaaagcttaaggactatggagtggatctcattgaagtttcagacaatggatgtggggtagaagaagaaaactttgaaggcttaatctctttcagctctgaaacatcacacatgtaagattcaagagtttgccgacctaactgaagttgaaactttcggttttcagggggaagctctgagctcactgtgtgcactgagcgatgtcaccatttctacctgccacgcgttggtgaaggttgggactcgactggtgtttgatcacgatgggaaaatcatccaggaaaccccctacccccaccccagagggaccacagtcagcgtgaagcagttattttctacgctacctgtgcgccataaggaatttcaaaggaatattaagaagacgtgcctgcttccccttcgccttctgccgtgattgtcagtttcctgaggcctccccagccatgcttcctgtacagcctgcagaactgtgagtcaattaaacctcttttcttcataaattaaaaaaaaa

SEQ ID NO: 41 - protein, 264 aa, Homo sapiens
MCPWRPRLGRRCMVSPREADLGPQKDTRLDLPRSPARAPREQNSLGEVDRRGPREQTRAPATAAPPRPLGSRGAEAAEPQEGLSATVSACFQEQQEMNTLQGPVSFKDVAVDFTQEEWRQLDPDEKIAYGDVMLENYSHLVSVGYDYHQAKHHHGVEVKEVEQGEEPWIMEGEFPCQHSPEPAKAIKPIDRKSVHQICSGPVVLSLSTAVKELVENSLDAGATNIDLKLKDYGVDLIEVSDNGCGVEEENFEGLISFSSETSHM

SEQ ID NO: 42 - DNA, 795 bp, Homo sapiens
atgtgtccttggcggcctagactaggccgtcgctgtatggtgagccccagggaggcggatctgggcccccagaaggacacccgcctggatttgccccgtagcccggcccgggcccctcgggagcagaacagccttggtgaggtggacaggaggggacctcgcgagcagacgcgcgcgccagcgacagcagccccgccccggcctctcgggagccggggggcagaggctgcggagccccaggagggtctatcagccacagtctctgcatgtttccaagagcaacaggaaatgaacacattgcaggggccagtgtcattcaaagatgtggctgtggatttcacccaggaggagtggcggcaactggaccctgatgagaagatagcatacggggatgtgatgttggagaactacagccatctagtttctgtggggtatgattatcaccaagccaaacatcatcatggagtggaggtgaaggaagtggagcagggagaggagccgtggataatggaaggtgaatttccatgtcaacatagtccagaacctgctaaggccatcaaacctattgatcggaagtcagtccatcagatttgctctgggccagtggtactgagtctaagcactgcagtgaaggagttagtagaaaacagtctggatgctggtgccactaatattgatctaaagcttaaggactatggagtggatctcattgaagtttcagacaatggatgtggggtagaagaagaaaactttgaaggcttaatctctttcagctctgaaacatcacacatgtaa

SEQ ID NO: 43 - protein, 264 aa, Homo sapiens; identical to SEQ ID NO: 41

SEQ ID NO: 44 - DNA, 2772 bp, Arabidopsis thaliana
atgcaaggagattcttctccgtctccgacgactactagctctcctttgataagacctataaacagaaacgtaattcacagaatctgttccggtcaagtcatcttagacctctcttcggccgtcaaggagcttgtcgagaatagtctcgacgccggcgccaccagtatagagattaacctccgagactacggcgaagactattttcaggtcattgacaatggttgtggcatttccccaaccaatttcaaggttcttgcacttaagcatcatacttctaaattagaggatttcacagatcttttgaatttgactacttatggttttagaggagaagccttgagctctctctgtgcattgggaaatctcactgtggaaacaagaacaaagaatgagccagttgctacgctcttgacgtttgatcattctggtttgcttactgctgaaaagaagactgctcgccaaattggtaccactgtcactgttaggaagttgttctctaatttacctgtacgaagcaaagagtttaagcggaatatacgcaaagaatatgggaagcttgtatctttattgaacgcatatgcgcttattgcgaaaggagtgcggtttgtctgctctaacacgactgggaaaaacccaaagtctgttgtgctgaacacacaagggaggggttcacttaaagataatatcataacagttttcggcattagtacctttacaagtctacagcctgtaagtatatgtgtatcagaagattgtagagttgaagggtttctttccaagcctggacagggtactggacgcaatttagcagatcgacagtatttctttataaatggtcggcctgtagatatgccaaaagtcagcaagttggtgaatgagttatataaagatacaagttctcggaaatatccagttaccattctggattttattgtgcctggtggagcatgtgatttgaatgtcacgcccgataaaagaaaggtgttcttttctgacgagacttctgttatcggttctttgagggaaggtctgaacgagatatattcctccagtaatgcgtcttatattgttaataggttcgaggagaattcggagcaaccagataaggctggagtttcgtcgtttcagaagaaatcaaatcttttgtcagaagggatagttctggatgtcagttctaaaacaagactaggggaagctattgagaaagaaaatccatccttaagggaggttgaaattgataatagttcgccaatggagaagtttaagtttgagatcaaggcatgtgggacgaagaaaggggaaggttctttatcagtccatgatgtaactcaccttgacaagacacctagcaaaggtttgcctcagttaaatgtgactgagaaagttactgatgcaagtaaagacttgagcagccgctctagctttgcccagtcaactttgaatacttttgttaccatgggaaaaagaaaacatgaaaacataagcaccatcctctctgaaacacctgtcctcagaaaccaaacttctagttatcgtgtggagaaaagcaaatttgaagttcgtgccttagcttcaaggtgtctcgtggaaggcgatcaacttgatgatatggtcatctcaaaggaagatatgacaccaagcgaaagagattctgaactaggcaatcggatttctcctggaacacaagctgataatgttgaaagacatgagagagaacatgaaaagcctataaggtttgaagaaccaacatcagataacacactcaccaagggggatgtggaaagggtttcagaggacaatccacggtgcagtcagccactgcgatctgtggccacagtgctggattccccagctcagtcaaccggtcctaaaatgttttccacattagaatttagtttccaaaacctcaggacaaggaggttagagaggctgtcgagattgcagtccacaggttatgtatctaaatgtatgaatacgccacagcctaaaaagtgctttgccgctgcaacattagagttatctcaaccggatgatgaagagcgaaaagcaagggctttagctgcagctacttctgagctggaaaggctttttcgaaaagaggatttcaggagaatgcaggtactcgggcaattcaatcttgggttcatcattgcaaaattggagcgagatctgttcattgtggatcagcatgcagctgatgagaaattcaacttcgaacatttagcaaggtcaactgtcctgaaccagcaacccttactccagcctttgaacttggaactctctccagaagaagaagtaactgtgttaatgcacatggatattatcagggaaaatggctttcttctagaggagaatccaagtgctcctcccggaaaacactttagactacgagccattccttatagcaagaatatcacctttggagtcgaagatcttaaagacctgatctcaactctaggagataaccatggggaatgttcggttgctagtagctacaaaaccagcaaaacagattcgatttgtccatcacgagtccgtgcaatgctagcatcccgagcatgcagatcatctgtgatgatcggagatccactcagaaaaaacgaaatgcagaagatagtagaacacttggcagatctcgaatctccttggaattgcccacacggacgaccaacaatgcgtcatcttgtggacttgacaactttactcacattacctgatgacgacaatgtcaatgatgatgatgatgatgatgcaaccatctcattggcatga

SEQ ID NO: 45 - protein, 923 aa, Arabidopsis thaliana
MQGDSSPSPTTTSSPLIRPINRNVIHRICSGQVILDLSSAVKELVENSLDAGATSIEINLRDYGEDYFQVIDNGCGISPTNFKVLALKHHTSKLEDFTDLLNLTTYGFRGEALSSLCALGNLTVETRTKNEPVATLLTFDHSGLLTAEKKTARQIGTTVTVRKLFSNLPVRSKEFKRNIRKEYGKLVSLLNAYALIAKGVRFVCSNTTGKNPKSVVLNTQGRGSLKDNIITVFGISTFTSLQPVSICVSEDCRVEGFLSKPGQGTGRNLADRQYFFINGRPVDMPKVSKLVNELYKDTSSRKYPVTILDFIVPGGACDLNVTPDKRKVFFSDETSVIGSLREGLNEIYSSSNASYIVNRFEENSEQPDKAGVSSFQKKSNLLSEGIVLDVSSKTRLGEAIEKENPSLREVEIDNSSPMEKFKFEIKACGTKKGEGSLSVHDVTHLDKTPSKGLPQLNVTEKVTDASKDLSSRSSFAQSTLNTFVTMGKRKHENISTILSETPVLRNQTSSYRVEKSKFEVRALASRCLVEGDQLDDMVISKEDMTPSERDSELGNRISPGTQADNVERHEREHEKPIRFEEPTSDNTLTKGDVERVSEDNPRCSQPLRSVATVLDSPAQSTGPKMFSTLEFSFQNLRTRRLERLSRLQSTGYVSKCMNTPQPKKCFAAATLELSQPDDEERKARALAAATSELERLFRKEDFRRMQVLGQFNLGFIIAKLERDLFIVDQHAADEKFNFEHLARSTVLNQQPLLQPLNLELSPEEEVTVLMHMDIIRENGFLLEENPSAPPGKHFRLRAIPYSKNITFGVEDLKDLISTLGDNHGECSVASSYKTSKTDSICPSRVRAMLASRACRSSVMIGDPLRKNEMQKIVEHLADLESPWNCPHGRPTMRHLVDLTTLLTLPDDDNVNDDDDDDATISLA

SEQ ID NO: 46 - DNA, 3466 bp, Arabidopsis thaliana
ttcgaattctctcagctcaaaacatcgtttctctctcactctctctcacaattccaaaaaatgcagcgccagagatcgattttgtctttcttccaaaaacccacggcggcgactacgaagggtttggtttccggcgatgctgctagcggcgggggcggcagcggaggaccacgatttaatgtgaaggaaggggatgctaaaggcgacgcttctgtacgttttgctgtttcgaaatctgtcgatgaggttagaggaacggatactccaccggagaaggttccgcgtcgtgtcctgccgtctggatttaagccggctgaatccgccggtgatgcttcgtccctgttctccaatattatgcataagtttgtaaaagtcgatgatcgagattgttctggagagaggagccgagaagatgttgttccgctgaatgattcatctctatgtatgaaggctaatgatgttattcctcaatttcgttccaataatggtaaaactcaagaaagaaaccatgcttttagtttcagtgggagagctgaacttagatcagtagaagatataggagtagatggcgatgttcctggtccagaaacaccagggatgcgtccacgtgcttctcgcttgaagcgagttctggaggatgaaatgacttttaaggaggataaggttcctgtattggactctaacaaaaggctgaaaatgctccaggatccggtttgtggagagaagaaagaagtaaacgaaggaaccaaatttgaatggcttgagtcttctcgaatcagggatgccaatagaagacgtcctgatgatcccctttacgatagaaagaccttacacataccacctgatgttttcaagaaaatgtctgcatcacaaaagcaatattggagtgttaagagtgaatatatggacattgtgcttttctttaaagtggggaaattttatgagctgtatgagctagatgcggaattaggtcacaaggagcttgactggaagatgaccatgagtggtgtgggaaaatgcagacaggttggtatctctgaaagtgggatagatgaggcagtgcaaaagctattagctcgtggatataaagttggacgaatcgagcagctagaaacatctgaccaagcaaaagccagaggtgctaatactataattccaaggaagctagttcaggtattaactccatcaacagcaagcgagggaaacatcgggcctgatgccgtccatcttcttgctataaaagagatcaaaatggagctacaaaagtgttcaactgtgtatggatttgcttttgttgactgtgctgccttgaggttttgggttgggtccatcagcgatgatgcatcatgtgctgctcttggagcgttattgatgcaggtttctccaaaggaagtgttatatgacagtaaagggctatcaagagaagcacaaaaggctctaaggaaatatacgttgacagggtctacggcggtacagttggctccagtaccacaagtaatgggggatacagatgctgctggagttagaaatataatagaatctaacggatactttaaaggttcttctgaatcatggaactgtgctgttgatggtctaaatgaatgtgatgttgcccttagtgctcttggagagctaattaatcatctgtctaggctaaagctagaagatgtacttaagcatggggatatttttccataccaagtttacaggggttgtctcagaattgatggccagacgatggtaaatcttgagatatttaacaatagctgtgatggtgtccttcagggacccttgaacaaatatcttgaaaactgtgttagtccaactggtaagcgactcttaaggaattggatctgccatccactcaaagatgtagaaagcatcaataaacggcttgatgtagttgaagaattcacggcaaactcagaaagtatgcaaatcactggccagtatctccacaaacttccagacttagaaagactgctcggacgcatcaagtctagcgttcgatcatcagcctctgtgttgcctgctcttctggggaaaaaagtgctgaaacaacgagttaaagcatttgggcaaattgtgaaagggttcagaagtggaattgatctgttgttggctctacagaaggaatcaaatatgatgagtttgctttataaactctgtaaacttcctatattagtaggaaaaagcgggctagagttatttctttctcaattcgaagcagccatagatagcgactttccaaattatcagaaccaagatgtgacagatgaaaacgctgaaactctcacaatacttatcgaactttttatcgaaagagcaactcaatggtctgaggtcattcacaccataagctgcctagatgtcctgagatcttttgcaatcgcagcaagtctctctgctggaagcatggccaggcctgttatttttcccgaatcagaagctacagatcagaatcagaaaacaaaagggccaatacttaaaatccaaggactatggcatccatttgcagttgcagccgatggtcaattgcctgttccgaatgatatactccttggcgaggctagaagaagcagtggcagcattcatcctcggtcattgttactgacgggaccaaacatgggcggaaaatcaactcttcttcgtgcaacatgtctggccgttatctttgcccaacttggctgctacgtgccgtgtgagtcttgcgaaatctccctcgtggatactatcttcacaaggcttggcgcatctgatagaatcatgacaggagagagtacctttttggtagaatgcactgagacagcgtcagttcttcagaatgcaactcaggattcactagtaatccttgacgaactgggcagaggaactagtactttcgatggatacgccattgcatactcggtttttcgtcacctggtagagaaagttcaatgtcggatgctctttgcaacacattaccaccctctcaccaaggaattcgcgtctcacccacgtgtcacctcgaaacacatggcttgcgcattcaaatcaagatctgattatcaaccacgtggttgtgatcaagacctagtgttcttgtaccgtttaaccgagggagcttgtcctgagagctacggacttcaagtggcactcatggctggaataccaaaccaagtggttgaaacagcatcaggtgctgctcaagccatgaagagatcaattggggaaaacttcaagtcaagtgagctaagatctgagttctcaagtctgcatgaagactggctcaagtcattggtgggtatttctcgagtcgcccacaacaatgcccccattggcgaagatgactacgacactttgttttgcttatggcatgagatcaaatcctcttactgtgttcccaaataaatggctatgacataacactatctgaagctcgttaagtcttttgcttctctgatgtttattcctcttaaaaaatgcg

SEQ ID NO: 47 - protein, 1109 aa, Arabidopsis thaliana
MQRQRSILSFFQKPTAATTKGLVSGDAASGGGGSGGPRFNVKEGDAKGDASVRFAVSKSVDEVRGTDTPPEKVPRRVLPSGFKPAESAGDASSLFSNIMHKFVKVDDRDCSGERSREDVVPLNDSSLCMKANDVIPQFRSNNGKTQERNHAFSFSGRAELRSVEDIGVDGDVPGPETPGMRPRASRLKRVLEDEMTFKEDKVPVLDSNKRLKMLQDPVCGEKKEVNEGTKFEWLESSRIRDANRRRPDDPLYDRKTLHIPPDVFKKMSASQKQYWSVKSEYMDIVLFFKVGKFYELYELDAELGHKELDWKMTMSGVGKCRQVGISESGIDEAVQKLLARGYKVGRIEQLETSDQAKARGANTIIPRKLVQVLTPSTASEGNIGPDAVHLLAIKEIKMELQKCSTVYGFAFVDCAALRFWVGSISDDASCAALGALLMQVSPKEVLYDSKGLSREAQKALRKYTLTGSTAVQLAPVPQVMGDTDAAGVRNIIESNGYFKGSSESWNCAVDGLNECDVALSALGELINHLSRLKLEDVLKHGDIFPYQVYRGCLRIDGQTMVNLEIFNNSCDGVLQGPLNKYLENCVSPTGKRLLRNWICHPLKDVESINKRLDVVEEFTANSESMQITGQYLHKLPDLERLLGRIKSSVRSSASVLPALLGKKVLKQRVKAFGQIVKGFRSGIDLLLALQKESNMMSLLYKLCKLPILVGKSGLELFLSQFEAAIDSDFPNYQNQDVTDENAETLTILIELFIERATQWSEVIHTISCLDVLRSFAIAASLSAGSMARPVIFPESEATDQNQKTKGPILKIQGLWHPFAVAADGQLPVPNDILLGEARRSSGSIHPRSLLLTGPNMGGKSTLLRATCLAVIFAQLGCYVPCESCEISLVDTIFTRLGASDRIMTGESTFLVECTETASVLQNATQDSLVILDELGRGTSTFDGYAIAYSVFRHLVEKVQCRMLFATHYHPLTKEFASHPRVTSKHMACAFKSRSDYQPRGCDQDLVFLYRLTEGACPESYGLQVALMAGIPNQVVETASGAAQAMKRSIGENFKSSELRSEFSSLHEDWLKSLVGISRVAHNNAPIGEDDYDTLFCLWHEIKSSYCVPK

SEQ ID NO: 48 - DNA, 5307 bp, Arabidopsis thaliana
aaagataagttcatacgacttttgtggctcatcaaaggccatcatcgtcctctatatacaatttagtgctttatagtacaaaaccttccacttccctttgtccaaagttttccaatttaatttataaacaggaataatattatctatataataaagtgaaaaataactatcattgtccaaataatttggtcgttgatcatgttactacaaagaaatgaaatccttagtagaagtatatatatatatatatttgtaacacactcaaaatggtaggtgttgttacagacagatgttcgttagcccagtaagcccaatatgagatttaatgggccttgatattttatagaccaaacattgaaacattgcacgcctggtctcaaagaacgttaatacacgcgccgccggttgccgccaatccgctttcccgccaaattcgacaccataaatttcttctagtcgctttcgattccagttccactgaaaaaccacgaaagaagaacatttgcaccgtagttgcagaaggtaggtgaaggatttagctttctctatcttccaatggagggtaatttcgaggaacagaacaagcttccggagctgaaattgggtaatgttaaaccctagttttttttttctttctcattttcgtattcgatttcccaattgggtttatgggttttgtaaaaggtctgatatttgttatgcattttttttttaatttttggaagatgcaaagcaagctcaagggtttctctcgttctacaaaaccctaccaaatgtaagttctcgttttctttcgatttctgggagaagttagagcttgtacagtgcctctaattgcaataaataacaccaattctagtcggaaagtagatgctttaaaattagggtttgaagcaattgtagacattttgttcattgggaagcgaattaggaaaaaaggcttaagattttttagcaatttctcgatctttgcttatgtgggttttgattgttctttgcttcaggatacgagagctgttagattctttgatcgcaaggtgagttcattgttctcaaatggtctagactttggttgtttaaatgtcgtcattgatttatggaaattttttgaatgcatttgcaggattattatacagctcatggtgaaaattcagttttcattgcaaagacttattatcatacaaccactgctctacgtcagctcgggagtggttcaaatgctctttcaagcgtaagcattagtaggaacatgttcgaaacgattgctagggatcttctcctggagcgtaatgatcatactgtagaactttatgaaggaagcggatcgaattggagacttgtgaaaacaggttctcctggaaacattggaagctttgaagatgttttgtttgcaaacaatgaaatgcaggacacaccagttgttgtctccatatttccaagttttcacgatggcagatgcgttattgggatggcctatgttgatctgactaggcgagttcttggactagctgagtttcttgatgatagccgcttcaccaatctggagtcttcgttgattgctctaggcgcaaaagaatgcatttttccagctgaatccggcaaatccaatgaatgcaaaagcctgtatgattccctggagaggtgtgccgtgatgataacagagaggaagaaacacgagttcaaaggaagagatttagattcagatcttaagagattggtgaaggggaatattgagcctgttagagatttggtatccgggtttgaccttgcgactcctgctctaggtgcattactctcgttttctgaacttctctcaaatgaggataactatgggaacttcacaatccgcagatatgatattggcggattcatgagacttgactctgcagctatgagggcgttgaatgtgatggagagcaaaactgatgctaataagaatttcagtttgtttggtctcatgaacagaacatgtaccgcagggatgggtaagagactgcttcatatgtggctgaagcaacccctcgtggatttgaatgagattaagacgagattagatatagttcagtgctttgttgaagaagctgggttaaggcaggatcttagacagcatctgaagcgaatctcagatgttgagaggcttttgcgcagtctcgagagaagaagaggtgggttacagcacattattaaactctatcaggtactttccgcacttcaatctgcttctctcaatgttaacaaaattgcattttcattgtcctaaatgtgtttatgcaactctgaagttataggtatgttattaagttcattactaattaagtcttcatcttttctctgcagtcagctataaggcttcccttcatcaaaacagctatgcaacagtacaccggagaattcgcatcactcatcagcgagaggtacctgaaaaagcttgaggctttatcagatcaagatcaccttggaaagttcatcgatttggttgagtgctctgtagatcttgaccagctagaaaatggagaatacatgatatcttcaaactacgacaccaaattggcatctctgaaagatcagaaagaattgctggagcagcagattcacgaattgcacaaaaagacagcgatagaacttgatcttcaggtcgacaaggctcttaaacttgacaaagcagcgcaatttgggcatgtcttcaggatcacgaagaaggaagagccaaagatcaggaagaagctgacgacacagtttatagtgctggagactcgcaaagacggagtgaagttcacaaacacaaagctaaaaaaactgggcgaccagtaccaaagtgttgtggatgattataggagctgtcaaaaggagctcgttgatcgtgtagttgagactgttaccagcttctctgaggtatgtttagttattcatattaagcattggactgttacagaattggttgtttaaaatcatagtaaactatatgtggaatttatatgtatattgtatggttataggtatttgaggacttagctgggttactttctgaaatggatgttttgttaagctttgctgatttggctgccagttgccctactccatactgtaggccagaaatcacctctttggttagtacaatctcaagttgattattttgttctgaaaatgaatagttttttctttccaagtttatgacataatgttgagagcacggttaataaattgtaggatgctggagatattgtactagaaggaagcagacatccatgtgtagaagctcaagattgggtgaatttcataccaaatgattgcagactcgtaagtattgaatgtggtaaataaactgagacgtctttgtttttcttgtttcccttttgacttgaacaaatacttgtttgccctttactgttctttgaaatcagatgagagggaagagttggtttcaaatagtaacagggcctaacatgggagggaagtccactttcatccgccaggtatgatgatttcctctagttcagttttgcttcatagacgtatgactaaagtcggtttccggccattataaatcccaggttggtgtgattgtgctgatggctcaagttggttcctttgttccttgtgataaagcatcaatttccataagagactgcatctttgcccgtgtaggagcaggcgattgccaagtgagtttaagtttagccctcaatgaacgaaaaactgctgatatcctgaacacccttattccaactttttttcctttggtgtgttagctgcgtggagtgtcaacttttatgcaagaaatgcttgaaaccgcatcgatattgaaaggcgctactgataagtcactgataattatcgatgaacttggtcgtggaacatcaacttatgatggttttggttagtttctctgcaatttctcttctttcatttggatgtttttagtaagttttctattatatattcatttttatggtcatatgtgagatttcagtgctcttgacatcatcgtggtgaatatatcaggtttagcttgggctatatgtgagcatctggttcaagtgaaaagagcaccaactctgtttgctactcacttccatgaacttactgccttggctcaagcaaactctgaggtctctggtaacactgttggtgtggcaaacttccatgtcagcgctcacattgacactgaaagccgcaaactcaccatgctttacaaggtctggtttataaattaaaaaattgctgatctgttgcagttaaaagtgtctctgtttttatgtttaatctaaattacttatttgattttcttacaaagatgaaattgaaattaattttgtgtggtgtgttgtttgtctggttaggttgaaccaggggcctgtgaccagagctttgggattcatgtggcggaatttgccaacttccctgaaagcgtcgtggccctcgcaagagagaaagctgcagagctggaagatttctctccctcctcgatgataatcaacaatgaggtcttgattcatttccccctttgtttttggttgatgatggaatcattctatcattcacccattctgcagtttatgctatattattataaatctatgtgacaaagatttaattctcgtattgttgtttgcaggagagtgggaagagaaagagcagagaagatgatccagatgaagtatcaagaggggcagagcgagctcacaagtttctgaaagagtttgcagcgatgccacttgataaaatggagcttaaagattcacttcaacgggtacgtgagatgaaagatgagctagagaaagatgctgcagactgccactggctcaggcagtttctgtgaagaacccctgacgttttttggtttttggttttgtaaatagcttaaatcggttcttgtagttgtggtcgttgcttgggatgaaactaaatgagggcaaaaacataattctacattttttgttagtaaagctcgttaatttactccctagtgctatcaattattttgcctattataattgttgatcaagtacttagagcaaccccaatggtttctaaacataagtttcttattttatagagagaaattttattataaaaaaatgtgtgggtttcttgattagtgaagaaaccatctccaaaataccttatattcttatataaggtattttggagagaatttctaactattcaagaaacttacataattaaatactattatttttattgttttaatgttaagaaacttatatttaaaaaccaccaatggaattgctcttagctaccatacaaataattataaaaatatatcgaaaagtagaagagccattt

SEQ ID NO: 49 - protein, 937 aa, Arabidopsis thaliana
MEGNFEEQNKLPELKLDAKQAQGFLSFYKTLPNDTRAVRFFDRKDYYTAHGENSVFIAKTYYHTTTALRQLGSGSNALSSVSISRNMFETIARDLLLERNDHTVELYEGSGSNWRLVKTGSPGNIGSFEDVLFANNEMQDTPVVVSIFPSFHDGRCVIGMAYVDLTRRVLGLAEFLDDSRFTNLESSLIALGAKECIFPAESGKSNECKSLYDSLERCAVMITERKKHEFKGRDLDSDLKRLVKGNIEPVRDLVSGFDLATPALGALLSFSELLSNEDNYGNFTIRRYDIGGFMRLDSAAMRALNVMESKTDANKNFSLFGLMNRTCTAGMGKRLLHMWLKQPLVDLNEIKTRLDIVQCFVEEAGLRQDLRQHLKRISDVERLLRSLERRRGGLQHIIKLYQSAIRLPFIKTAMQQYTGEFASLISERYLKKLEALSDQDHLGKFIDLVECSVDLDQLENGEYMISSNYDTKLASLKDQKELLEQQIHELHKKTAIELDLQVDKALKLDKAAQFGHVFRITKKEEPKIRKKLTTQFIVLETRKDGVKFTNTKLKKLGDQYQSVVDDYRSCQKELVDRVVETVTSFSEVFEDLAGLLSEMDVLLSFADLAASCPTPYCRPEITSLDAGDIVLEGSRHPCVEAQDWVNFIPNDCRLMRGKSWFQIVTGPNMGGKSTFIRQVGVIVLMAQVGSFVPCDKASISIRDCIFARVGAGDCQLRGVSTFMQEMLETASILKGATDKSLIIIDELGRGTSTYDGFGLAWAICEHLVQVKRAPTLFATHFHELTALAQANSEVSGNTVGVANFHVSAHIDTESRKLTMLYKVEPGACDQSFGIHVAEFANFPESVVALAREKAAELEDFSPSSMIINNEESGKRKSREDDPDEVSRGAERAHKFLKEFAAMPLDKMELKDSLQRVREMKDELEKDAADCHWLRQFL

SEQ ID NO: 50 - DNA, 3521 bp, Arabidopsis thaliana
ctaagaaagcgcgcgaaaattggcaacccaagttcgccatagccacgaccacgaccttccatttctcttaaacggaggagattacgaataaagcaattatgggcaagcaaaagcagcagacgatttctcgtttcttcgctcccaaacccaaatccccgactcacgaaccgaatccggtagccgaatcatcaacaccgccaccgaagatatccgccactgtatccttctctccttccaagcgtaagcttctctccgaccacctcgccgccgcgtcacccaaaaagcctaaactttctcctcacactcaaaacccagtacccgatcccaatttacaccaaagatttctccagagatttctggaaccctcgccggaggaatatgttcccgaaacgtcatcatcgaggaaatacacaccattggaacagcaagtggtggagctaaagagcaagtacccagatgtggttttgatggtggaagttggttacaggtacagattcttcggagaagacgcggagatcgcagcacgcgtgttgggtatttacgctcatatggatcacaatttcatgacggcgagtgtgccaacatttcgattgaatttccatgtgagaagactggtgaatgcaggatacaagattggtgtagtgaagcagactgaaactgcagccattaagtcccatggtgcaaaccggaccggcccttttttccggggactgtcggcgttgtataccaaagccacgcttgaagcggctgaggatataagtggtggttgtggtggtgaagaaggttttggttcacagagtaatttcttggtttgtgttgtggatgagagagttaagtcggagacattaggctgtggtattgaaatgagttttgatgttagagtcggtgttgttggcgttgaaatttcgacaggtgaagttgtttatgaagagttcaatgataatttcatgagaagtggattagaggctgtgattttgagcttgtcaccagctgagctgttgcttggccagcctctttcacaacaaactgagaagtttttggtggcacatgctggacctacctcaaacgttcgagtggaacgtgcctcactggattgtttcagcaatggtaatgcagtagatgaggttatttcattatgtgaaaaaatcagcgcaggtaacttagaagatgataaagaaatgaagctggaggctgctgaaaaaggaatgtcttgcttgacagttcatacaattatgaacatgccacatctgactgttcaagccctcgccctaacgttttgccatctcaaacagtttggatttgaaaggatcctttaccaaggggcctcatttcgctctttgtcaagtaacacagagatgactctctcagccaatactctgcaacagttggaggttgtgaaaaataattcagatggatcggaatctggctccttattccataatatgaatcacacacttacagtatatggttccaggcttcttagacactgggtgactcatcctctatgcgatagaaatttgatatctgctcggcttgatgctgtttctgagatttctgcttgcatgggatctcatagttcttcccagctcagcagtgagttggttgaagaaggttctgagagagcaattgtatcacctgagttttatctcgtgctctcctcagtcttgacagctatgtctagatcatctgatattcaacgtggaataacaagaatctttcatcggactgctaaagccacagagttcattgcagttatggaagctattttacttgcggggaagcaaattcagcggcttggcataaagcaagactctgaaatgaggagtatgcaatctgcaactgtgcgatctactcttttgagaaaattgatttctgttatttcatcccctgttgtggttgacaatgccggaaaacttctctctgccctaaataaggaagcggctgttcgaggtgacttgctcgacatactaatcacttccagcgaccaatttcctgagcttgctgaagctcgccaagcagttttagtcatcagggaaaagctggattcctcgatagcttcatttcgcaagaagctcgctattcgaaatttggaatttcttcaagtgtcggggatcacacatttgatagagctgcccgttgattccaaggtccctatgaattgggtgaaagtaaatagcaccaagaagactattcgatatcatcccccagaaatagtagctggcttggatgagctagctctagcaactgaacatcttgccattgtgaaccgagcttcgtgggatagtttcctcaagagtttcagtagatactacacagattttaaggctgccgttcaagctcttgctgcactggactgtttgcactccctttcaactctatctagaaacaagaactatgtccgtcccgagtttgtggatgactgtgaaccagttgagataaacatacagtctggtcgtcatcctgtactggagactatattacaagataacttcgtcccaaatgacacaattttgcatgcagaaggggaatattgccaaattatcaccggacctaacatgggaggaaagagctgctatatccgtcaagttgctttaatttccataatggctcaggttggttcctttgtaccagcgtcattcgccaagctgcacgtgcttgatggtgttttcactcggatgggtgcttcagacagtatccagcatggcagaagtacctttctagaagaattaagtgaagcgtcacacataatcagaacctgttcttctcgttcgcttgttatattagatgagcttggaagaggcactagcacacacgacggtgtagccattgcctatgcaacattacagcatctcctagcagaaaagagatgtttggttctttttgtcacgcattaccctgaaatagctgagatcagtaacggattcccaggttctgttgggacataccatgtctcgtatctgacattgcagaaggataaaggcagttatgatcatgatgatgtgacctacctatataagcttgtgcgtggtctttgcagcaggagctttggttttaaggttgctcagcttgcccagatacctccatcatgtatacgtcgagccatttcaatggctgcaaaattggaagctgaggtacgtgcaagagagagaaatacacgcatgggagaaccagaaggacatgaagaaccgagaggcgcagaagaatctatttcggctctaggtgacttgtttgcagacctgaaatttgctctctctgaagaggacccttggaaagcattcgagtttttaaagcatgcttggaagattgctggcaaaatcagactaaaaccaacttgttcattttgatttaatcttaacattatagcaactgcaaggtcttgatcatctgttagttgcgtactaacttatgtgtattagtataacaagaaaagagaattagagagatggattctaatccggtgttgcagtacatcttttctccacccgcataaaaaaaaaaaaaaaaaaaaaaaaaaaaaaaaa

SEQ ID NO: 51 - protein, 1081 aa, Arabidopsis thaliana
MGKQKQQTISRFFAPKPKSPTHEPNPVAESSTPPPKISATVSFSPSKRKLLSDHLAAASPKKPKLSPHTQNPVPDPNLHQRFLQRFLEPSPEEYVPETSSSRKYTPLEQQVVELKSKYPDVVLMVEVGYRYRFFGEDAEIAARVLGIYAHMDHNFMTASVPTFRLNFHVRRLVNAGYKIGVVKQTETAAIKSHGANRTGPFFRGLSALYTKATLEAAEDISGGCGGEEGFGSQSNFLVCVVDERVKSETLGCGIEMSFDVRVGVVGVEISTGEVVYEEFNDNFMRSGLEAVILSLSPAELLLGQPLSQQTEKFLVAHAGPTSNVRVERASLDCFSNGNAVDEVISLCEKISAGNLEDDKEMKLEAAEKGMSCLTVHTIMNMPHLTVQALALTFCHLKQFGFERILYQGASFRSLSSNTEMTLSANTLQQLEVVKNNSDGSESGSLFHNMNHTLTVYGSRLLRHWVTHPLCDRNLISARLDAVSEISACMGSHSSSQLSSELVEEGSERAIVSPEFYLVLSSVLTAMSRSSDIQRGITRIFHRTAKATEFIAVMEAILLAGKQIQRLGIKQDSEMRSMQSATVRSTLLRKLISVISSPVVVDNAGKLLSALNKEAAVRGDLLDILITSSDQFPELAEARQAVLVIREKLDSSIASFRKKLAIRNLEFLQVSGITHLIELPVDSKVPMNWVKVNSTKKTIRYHPPEIVAGLDELALATEHLAIVNRASWDSFLKSFSRYYTDFKAAVQALAALDCLHSLSTLSRNKNYVRPEFVDDCEPVEINIQSGRHPVLETILQDNFVPNDTILHAEGEYCQIITGPNMGGKSCYIRQVALISIMAQVGSFVPASFAKLHVLDGVFTRMGASDSIQHGRSTFLEELSEASHIIRTCSSRSLVILDELGRGTSTHDGVAIAYATLQHLLAEKRCLVLFVTHYPEIAEISNGFPGSVGTYHVSYLTLQKDKGSYDHDDVTYLYKLVRGLCSRSFGFKVAQLAQIPPSCIRRAISMAAKLEAEVRARERNTRMGEPEGHEEPRGAEESISALGDLFADLKFALSEEDPWKAFEFLKHAWKIAGKIRLKPTCSF

SEQ ID NO: 52 - DNA, 7080 bp, Arabidopsis thaliana
ctcttcgccgactgtttcactccccttctctctcactctctgtgcgctttattccactctccgatggctccgtctcgccgacagatcagcggaagatctccgttggtgaaccagcagcgtcaaatcacctccttctttgggaaatctgcttcatcatcttcttctccgtctccatctccttcaccatctctctccaataagaaaacccccaaatctaacaaccctaaccctaaatctccgtctccgtcaccatctccgcctaagaaaacccccaaattgaaccctaaccctagttctaatcttcctgctcgtagtcctagccctggtcctgatactccttctcctgtacagtccaagtttaagaagccccttctcgtcatcggacagacaccttcgcctcctcaatcggtggtaattacttacggtgacgaggtggtggggaagcaagttagggtttattggcctttggataaaaaatggtatgatgggagcgtgacgttttatgataagggtgagggtaagcatgtggttgagtatgaagatggggaagaagagtctttggatttgggaaaggagaagactgagtgggtggttggggaaaaatcaggagataggtttaatcgattgaaacgaggcgcttcggctttgagaaaagttgtgacggatagtgatgatgatgtggagatgggtaatgtggaagaagataaaagtgacggtgatgattctagcgatgaggattggggaaagaatgttgggaaggaggtttgtgagagtgaagaagatgatgtggagttggttgatgagaatgaaatggatgaagaagagttggtggaagagaaagatgaagaaacttctaaagttaatagagtatccaaaactgactctagaaagcggaagactagtgaagtaacgaaatcaggtggtgagaagaaaagcaagactgatacaggcactatcttgaaaggttttaaggcttctgttgtggagcctgcgaagaagattggacaaggtaaaccgaagagtctcttgttgtaatcatatgcttgtatttgcattgttttagtttgtggtatgtctcttgcactgacttttgtttcagatagtgtatgttgttggttgcttaatattatttgtgtcttactacagctgatagggtggtcaagggtttggaagataacgtgttggatggggatgctcttgctagatttggtgctcgtgattctgagaaattccgctttttgggagtgtaagtctttcacaaaaaaaattccatcttagaggctatttgctacggtggttaggagtagagaatgtaaatttgtgtcttaagcaatattgacttctctactggcaggagcatctctggttttcttttatcttcatgatgtattagtaggctgcatgatccctattctagctaagttagttctgttaattatttttggtgaacagagaccgaagggatgctaaaaggagacgccctactgatgagaattatgatccgaggacactctacctccctcctgattttgtgaaaaaattaactggaggccaggtcagaagagcgcatggaaatctggttcaggatttttggtgaagctaatcaactttcacttatatgattttgtggccttttttcagagacaatggtgggagtttaaagcaaagcatatggacaaagttgtattcttcaaggtagaacgataattacttatttcgttataacttatttattgatgggagattctaggataaatggtcttcttttgtggcaagcagatgggtaaattctatgagctttttgagatggatgcacatgtcggagctaaggaactggatatacaatacatgaaggtaactgtttgttatgactcataactaggtgatgcatttgaagacatctgttaaaaatgttaaaaaaccgaaaatttggcatcagattatgctaaaagggttcttttcattggtgttacattacaaatttctcctgtattgtctctaatgtatctctctttacaagcccctgacatatgcatttattttgtagggagagcaacctcattgtggatttccggagaagaatttttctgtaaacattgagaaattagttagaaaggtttgtttccagaaatatagcaactccagttcaagcgtgatctatttcttgttacgtgtagagaaattacattcatggcaaatgctgtactttgggtagaaataaagttgattgaattgaatggaacagggctatcgggttttagttgtcgaacaaacagaaacacctgatcagctggagcaacgccgaaaagagacaggttccaaggataaagtatgtcccactatgaatctaatttagttggcattatcagttcaagtcaatttgtttgctcttgaaactaaaatttgttcactttgggtgatgcctatgtagaaaaattatgatagggagggctcatagtgacagaacttctgtttttataggttgtgaagcgcgaagtatgtgcagttgttacaaaaggcacgctgacagatggggagatgctattaactaatccggatgcatcttatctaatggccttgactgaaggaggagaaagtttaactaatcctacagcagagcacaattttggtgtatgtttggttgatgttgcgacacagaagataatactgggccaggtgagttctagttgatgaatggtacctggttgcacttatacgtaacatttctcggtgtatattgatggcatttttttttcattcgtaccagtttaaggatgatcaagattgcagtgcattatcttgcctgctatctgagatgaggccggtggaaattattaaaccagctaaggtgttgagttatgcaacagagagaacaatagttagacaaaccagaaatcccttagtaaataatctcgttccactttctgaattttgggattcggagaagaccatatatgaagttggaattatctacaagcgaatcaattgtcaaccgtcttctgcttattctagtgagggaaagattctaggtgatggttcaagctttcttccaaaaatgttgtctgaattagcaactgaagataagaatggtagcctggcactctctgctcttggtggtgccatttactacctgcgacaagcattcttggatgagagtctgcttagatttgcaaagtttgaatccctgccttactgtgatttcagcaacgttaatgagaagcagcacatggttcttgatgctgctgctcttgaaaaccttgagatatttgaaaacagtagaaatggaggctattcagggtaaagtttctctatcttaccatgtattattaaacataattgatgtgttctaaatctagagtgttgtcttttgaagaacgctgtatgctcaactgaatcaatgtatcactgcatctgggaaacggttactgaaaacatggctggcaagacctttatataatacggaactgatcaaggaacgacaagatgctgtagcaattctgcgggtgagtctttcaacaagttgtttgactttgctgctgtcatttctctgtctctcaactagacaataacttggcatcttggtttcacatttgatcatttttcatgtctgtttcgctatccatggatctctcctcagaattacactatttccccattatgggtgttcaagaccatttttgccactgtttcactggcaaagatgatgttttcctatgcgttcaactaaccatctatttctagaacttattccctaagattataaaacttactctgcttcttcagcatgtcaaggctttcgtttacactatccatctgacaatgtattatggtactgtcccttccctcagggtgaaaatcttccgtactcactggaattccggaagtcgttgtccagacttccagacatggaacggttgattgcacgtatgttttctagcatgtaagggattagctagattgagatgttaattcttacattatatgtttataccaaagacttactaaacatatttgttaaacttgtgttacgtgttatagtgaagctagtggaagaaatggcgataaagtggtgctatatgaagatacagctaagaagcaggtacaggaattcatatcaactctacgtggttgtgaaacaatggcagaagcatgctcttctctccgtgctatcttgaagcatgatacatccaggcggctgcttcatttactaactcctggtataatcaatttgctccatattcacattcttatactggcaaattgcacagcatctcatatcatttctctgccaggtcaaagtcttccaaatatatcatcctccataaagtatttcaaggatgcttttgactgggtagaagctcacaattctggacgtgtaataccccatgaaggagcagatgaagagtatgattgtgcctgcaaaacagtagaagaatttgagtccagtttgaaaaaacatctgaaagagcaacggaaattactcggagatgcatcagtgagaattacttcactatttttttttactccttaaatggctaatcaaccgagggttttctgatcagatctttggtgctcttttgtcttcttatccagataaactatgttacagttggaaaagatgaatacctcttggaagttcctgaaagtttaagtgggagtgttcctcatgattatgaattatgctcatcgaaaaaggtaaaagttgtaccaagtttcacattctaaagaaattggcatttcgctttcgtcataacaagtcgatagtcttctcgtaattgctgtctgctgatatatttactatatagagacccttaattttaaacatgagattttcttactttttactctctttcagggtgtctctcgatattggactcctaccataaagaaattattaaaagagctatcacaagcaaaatctgaaaaagagtcggccctgaagagcatttcacagagattgattggacgtttctgcgagcatcaagaaaaatggagacaattggtttctgcaacagctggtatggacaagttcatgttttaaaaaaaaaaaattgtttaaggaattttcagcatcttccttcagaatatgtatcttgcttatccaattcctgttaattactgtcacccagtgttagctttgtgggtcgtcgcttggacccttttcgttgtgaacatttgttgagctagttagaattgagtttgatcccacactttatagattgagttagaagtaggcatgcagaagaaaatgaatcttaggcagacgtatagttcaatcacatcttataagcaagaggtttcttgggtggaagattgttttatagaattaggcatgcaaacaactttgcacttagacctttatgtggatacatttttgacatgaattctttctattgcagagctggacgtgttgatcagcctcgcttttgcaagtgattcttatgaaggagtaagatgccgcccagtaatatctggttctacatctgatggtgttccacacttgtctgccactggtctagggcatccagttctaaggggtgattcgttaggcagaggctcttttgtaccaaataatgtaaagataggtggtgctgagaaagccagtttcatcctcctcacaggccctaatatgggtggaaaatcaacccttcttcgccaagtttgcttggctgtaatcttggctcaggtaagctatcatttgaaaaaactttgtaggcaatgggctttgacccgtttaattttgatgaaagaaactcaagcaatgatgatcttttcacagattggagcagatgtcccagcagaaacctttgaggtttcgcctgttgacaaaatttgtgtccggatgggtgcaaaagatcatatcatggcaggacaaagcacgtttttaacagaactttcagaaactgcggtaatgttggtaagtaatgttcattctgtttgtcaaattgattacatgaagctttctaagataaatgtgaaacttgccacagtggttacccttttgagagttggtcacaggctttgttaaactatgcgaatgccaacaaacgcactgatagaatgttttatattaataatatgcagacatcagccacccgaaactcgctggtggtgctagatgagcttggacgaggaacagccacatcagatgggcaagccattgcgtatgttgaatcaattattgcgtatcatgttttttgggacttactgttattgttcactttatctaaaatatcttaactatttacagggaatccgtacttgagcacttcatagaaaaggtgcagtgtagaggattcttctctactcattatcatcgtctctctgtggattatcaaaccaatccaaaggtattgtgaaaagtgtctgcttcagtttctgggtttgaaagacttgagaactatcaataataatctgattgtttgtgtacattctgaaacttgtcaaaaaccgatcagtcttgaatatttgtttggataggtctcactttgccatatggcatgtcaaataggagaaggaatcggtggagtagaagaagttacatttctctatagattgactcctggtgcatgtcctaaaagttatggagttaacgttgctcggttagctggtaagaacactgaattctctactccatcacctctactcagttaaacagaagcagtcactcatcaaattgttttggttttaatctccataggtcttccagattacgtactccagagagccgtgataaaatcccaagaattcgaggctttgtacggtaaaaaccatagaaaaaccgatcataaattagcagcaatgataaagcagatcatcagcagtgttgcatcagattctgattactcagcttcaaaggactcattgtgtgagctacactccatggccaatacatttctccggttaaccaactaatttaacagctctacgcctttccggtttgtcgttcttcttgtaactctttaaccaaggtcaatccacgagcttcgtcgtgtcaaatactaaaacctgagtcagcctgaaactaaactcctgagtagagactcagttttgaggtgtgggtttagcttctgagtcttt

SEQ ID NO: 53 - protein, 1324 aa, Arabidopsis thaliana
MAPSRRQISGRSPLVNQQRQITSFFGKSASSSSSPSPSPSPSLSNKKTPKSNNPNPKSPSPSPSPPKKTPKLNPNPSSNLPARSPSPGPDTPSPVQSKFKKPLLVIGQTPSPPQSVVITYGDEVVGKQVRVYWPLDKKWYDGSVTFYDKGEGKHVVEYEDGEEESLDLGKEKTEWVVGEKSGDRFNRLKRGASALRKVVTDSDDDVEMGNVEEDKSDGDDSSDEDWGKNVGKEVCESEEDDVELVDENEMDEEELVEEKDEETSKVNRVSKTDSRKRKTSEVTKSGGEKKSKTDTGTILKGFKASVVEPAKKIGQADRVVKGLEDNVLDGDALARFGARDSEKFRFLGVDRRDAKRRRPTDENYDPRTLYLPPDFVKKLTGGQRQWWEFKAKHMDKVVFFKMGKFYELFEMDAHVGAKELDIQYMKGEQPHCGFPEKNFSVNIEKLVRKGYRVLVVEQTETPDQLEQRRKETGSKDKVVKREVCAVVTKGTLTDGEMLLTNPDASYLMALTEGGESLTNPTAEHNFGVCLVDVATQKIILGQFKDDQDCSALSCLLSEMRPVEIIKPAKVLSYATERTIVRQTRNPLVNNLVPLSEFWDSEKTIYEVGIIYKRINCQPSSAYSSEGKILGDGSSFLPKMLSELATEDKNGSLALSALGGAIYYLRQAFLDESLLRFAKFESLPYCDFSNVNEKQHMVLDAAALENLEIFENSRNGGYSGTLYAQLNQCITASGKRLLKTWLARPLYNTELIKERQDAVAILRGENLPYSLEFRKSLSRLPDMERLIARMFSSIEASGRNGDKVVLYEDTAKKQVQEFISTLRGCETMAEACSSLRAILKHDTSRRLLHLLTPGQSLPNISSSIKYFKDAFDWVEAHNSGRVIPHEGADEEYDCACKTVEEFESSLKKHLKEQRKLLGDASINYVTVGKDEYLLEVPESLSGSVPHDYELCSSKKGVSRYWTPTIKKLLKELSQAKSEKESALKSISQRLIGRFCEHQEKWRQLVSATAELDVLISLAFASDSYEGVRCRPVISGSTSDGVPHLSATGLGHPVLRGDSLGRGSFVPNNVKIGGAEKASFILLTGPNMGGKSTLLRQVCLAVILAQIGADVPAETFEVSPVDKICVRMGAKDHIMAGQSTFLTELSETAVMLTSATRNSLVVLDELGRGTATSDGQAIAESVLEHFIEKVQCRGFFSTHYHRLSVDYQTNPKVSLCHMACQIGEGIGGVEEVTFLYRLTPGACPKSYGVNVARLAGLPDYVLQRAVIKSQEFEALYGKNHRKTDHKLAAMIKQIISSVASDSDYSASKDSLCELHSMANTFLRLTN

SEQ ID NO: 54 - DNA, 2340 bp, Arabidopsis thaliana
atgcaaggagattcttctccgtctccgacgactactagctctcctttgataagacctataaacagaaacgtaattcacagaatctgttccggtcaagtcatcttagacctctcttcggccgtcaaggagcttgtcgagaatagtctcgacgccggcgccaccagtatagagattaacctccgagactacggcgaagactattttcaggtcattgacaatggttgtggcatttccccaaccaatttcaaggtttgtgtccaaattctccgaagaacttttgatgttcttgcacttaagcatcatacttctaaattagaggatttcacagatcttttgaatttgactacttatggttttagaggagaagccttgagctctctctgtgcattgggaaatctcactgtggaaacaagaacaaagaatgagccagttgctacgctcttgacgtttgatcattctggtttgcttactgctgaaaagaagactgctcgccaaattggtaccactgtcactgttaggaagttgttctctaatttacctgtacgaagcaaagagtttaagcggaatatacgcaaagaatatgggaagcttgtatctttattgaacgcatatgcgcttattgcgaaaggagtgcggtttgtctgctctaacacgactgggaaaaacccaaagtctgttgtgctgaacacacaagggaggggttcacttaaagataatatcataacagttttcggcattagtacctttacaagtctacagcctggtactggacgcaatttagcagatcgacagtatttctttataaatggtcggcctgtagatatgccaaaagtcagcaagttggtgaatgagttatataaagatacaagttctcggaaatatccagttaccattctggattttattgtgcctggtggagcatgtgatttgaatgtcacgcccgataaaagaaaggtgttcttttctgacgagacttctgttatcggttctttgagggaaggtctgaacgagatatattcctccagtaatgcgtcttatattgttaataggttcgaggagaattcggagcaaccagataaggctggagtttcgtcgtttcagaagaaatcaaatcttttgtcagaagggatagttctggatgtcagttctaaaacaagactaggggaagctattgagaaagaaaatccatccttaagggaggttgaaattgataatagttcgccaatggagaagtttaagtttgagatcaaggcatgtgggacgaagaaaggggaaggttctttatcagtccatgatgtaactcaccttgacaagacacctagcaaaggtttgcctcagttaaatgtgactgagaaagttactgatgcaagtaaagacttgagcagccgctctagctttgcccagtcaactttgaatacttttgttaccatgggaaaaagaaaacatgaaaacataagcaccatcctctctgaaacacctgtcctcagaaaccaaacttctagttatcgtgtggagaaaagcaaatttgaagttcgtgccttagcttcaaggtgtctcgtggaaggcgatcaacttgatgatatggtcatctcaaaggaagatatgacaccaagcgaaagagattctgaactaggcaatcggatttctcctggaacacaagctgataatgttgaaagacatgagagagtactcgggcaattcaatcttgggttcatcattgcaaaattggagcgagatctgttcattgtggatcagcatgcagctgatgagaaattcaacttcgaacatttagcaaggtcaactgtcctgaaccagcaacccttactccagcctttgaacttggaactctctccagaagaagaagtaactgtgttaatgcacatggatattatcagggaaaatggctttcttctagaggagaatccaagtgctcctcccggaaaacactttagactacgagccattccttatagcaagaatatcacctttggagtcgaagatcttaaagacctgatctcaactctaggagataaccatggggaatgttcggttgctagtagctacaaaaccagcaaaacagattcgatttgtccatcacgagtccgtgcaatgctagcatcccgagcatgcagatcatctgtgatgatcggagatccactcagaaaaaacgaaatgcagaagatagtagaacacttggcagatctcgaatctccttggaattgcccacacggacgaccaacaatgcgtcatcttgtggacttgacaactttactcacattacctgatgacgacaatgtcaatgatgatgatgatgatgatgcaaccatctcattggcatga

SEQ ID NO: 55 - protein, 779 aa, Arabidopsis thaliana
MQGDSSPSPTTTSSPLIRPINRNVIHRICSGQVILDLSSAVKELVENSLDAGATSIEINLRDYGEDYFQVIDNGCGISPTNFKVCVQILRRTFDVLALKHHTSKLEDFTDLLNLTTYGFRGEALSSLCALGNLTVETRTKNEPVATLLTFDHSGLLTAEKKTARQIGTTVTVRKLFSNLPVRSKEFKRNIRKEYGKLVSLLNAYALIAKGVRFVCSNTTGKNPKSVVLNTQGRGSLKDNIITVFGISTFTSLQPGTGRNLADRQYFFINGRPVDMPKVSKLVNELYKDTSSRKYPVTILDFIVPGGACDLNVTPDKRKVFFSDETSVIGSLREGLNEIYSSSNASYIVNRFEENSEQPDKAGVSSFQKKSNLLSEGIVLDVSSKTRLGEAIEKENPSLREVEIDNSSPMEKFKFEIKACGTKKGEGSLSVHDVTHLDKTPSKGLPQLNVTEKVTDASKDLSSRSSFAQSTLNTFVTMGKRKHENISTILSETPVLRNQTSSYRVEKSKFEVRALASRCLVEGDQLDDMVISKEDMTPSERDSELGNRISPGTQADNVERHERVLGQFNLGFIIAKLERDLFIVDQHAADEKFNFEHLARSTVLNQQPLLQPLNLELSPEEEVTVLMHMDIIRENGFLLEENPSAPPGKHFRLRAIPYSKNITFGVEDLKDLISTLGDNHGECSVASSYKTSKTDSICPSRVRAMLASRACRSSVMIGDPLRKNEMQKIVEHLADLESPWNCPHGRPTMRHLVDLTTLLTLPDDDNVNDDDDDDATISLA

SEQ ID NO: 56 - DNA, 440 bp, Arabidopsis thaliana
atgcaaggagattcttctccgtctccgacgactactagctctcctttgataagacctataaacagaaacgtaattcacagaatctcttccggtcaagtcatcttagacctctcttcggccgtcaaggagcttgtcgagaatagtctcgacgcggcgccaccagtatagagattaacctccgagactacggcgaagactattttcaggtcattgacaatggttgtggcatttccccaaccaatttcaaggtttgtgtccaaattctccgaagaacttttgatgttcttgcacttaagcatcatacttctaaattagaggatttcacagatcttttgaatttgactacttatggttttagaggagaagccttgagctctctctgtgcattgggaaatctcactgtggaaacaagaacaaagaatgagccagttgctacgctc

SEQ ID NO: 57 - protein, 141 aa, Arabidopsis thaliana
MQGDSSPSPTTTSSPLIRPINRNVIHRISSGQVILDLSSAVKELVENSLDAAPPVRLTSETTAKTIFRSLTMVVAFPQPISRFVSKFSEELLMFLHLSIILLNRISQIFILLMVLEEKPALSVHWEISLWKQEQRMSQLLR

SEQ ID NO: 58 - DNA, 2501 bp, Oryza sativa
cggcacgagattttgcagtctcctctcctcctccgctcgagcgagtgagtcccgaccacgtcgctgccctcgcctcaccgccggccaaccgccgtgacgagagatcgagcagggcggggcatggacgagccttcgccgcgcggaggtgggtgcgccggggagccgccccgcatccggaggttggaggagtcggtggtgaaccgcatcgcggcgggggaggtgatccagcggccgtcgtcggcggtgaaggagctcatcgagaacagcctcgacgctggcgcctccagcgtctccgttgcggtgaaggacggtggcctcaagctcatccaggtctccgatgacggccatggcatcaggtttgaggatttggcaatattgtgcgaaaggcatactacctcaaagttatctgcatacgaggatctgcagaccataaaatcgatggggttcagaggggaggctttggctagtatgacttatgttggccatgttaccgtgacaacgataacagaaggccaattgcacggctacagggtttcttacagagatggtgtaatggagaatgagcctaagccttgcgctgcggtgaaaggaactcaagtcatggttgaaaatctattttacaacatggtagcccgcaagaaaacattgcagaactccaatgatgactaccccaagatcgtagacttcatcagtcggtttgcagtccatcacatcaacgttaccttctcttgcagaaagcatggagccaatagagcagatgttcatagtgcaagtacatcctcaaggttagatgctatcaggagtgtctatggggcttctgtcgttcgtgatctcatagaaataaaggtttcatatgaggatgctgcagattcaatcttcaagatggatggttacatctcaaatgcaaattatgtggcaaagaagattacaatgattcttttcataaatgataggcttgtagactgtactgctttgaaaagagctattgaatttgtgtactctgcaacattgcctcaagcatccaaacctttcatatacatgtccatacatcttccatcagaacacgtggatgttaatatacacccaaccaagaaagaggttagccttttgaatcaagagcgtattattgaaacaataagaaatgctattgaggaaaaactgatgaattctaatacaaccaggatattccaaactcaggcattaaacttatcagggattgctcaagctaacccacaaaaggataaggtttctgaggccagtatgggttctggaacaaaatctcaaaaaattcctgtgagccaaatggtcagaacagatccacgcaatccatctggaagattgcacacctactggcacgggcaatcttcaaatcttgaaaagaaatttgatcttgtatctgtaagaaatgttgtaagatcaaggagaaaccaaaaagatgctggtgatttgtcaagccgtcatgagctccttgtggaaatagattctagcttccatcctggccttttggacattgtcaagaactgcacatatgttggacttgccgatgaagcctttgctttgatacaacacaatacccgcttataccttgtaaatgtggtaaatattagtaaagaacttatgtaccagcaagctttgtgccgttttgggaacttcaatgctattcagctcagtgaaccagctccacttcaggagttgctggtgatggcactgaaagacgatgaattgatgagtgatgaaaaggatgatgagaaactggagattgcagaagtaaacactgagatactaaaagaaaatgctgagatgattaatgagtacttttctattcacattgatcaagatggcaaattgacaagacttcctgttgtactggaccagtacacccctgatatggaccgtcttccagaatttgtgttggctttaggaaatgatgttacttgggatgacgagaaagagtgcttcagaacagtagcttctgctgtaggaaacttctatgcacttcatcccccaatccttccaaatccatctgggaatggcattcatttatacaagaaaaatagagattcaatggctgatgaacatgctgagaatgatctaatatcagatgaaaatgacgttgatcaagaacttcttgcggaagcagaagcagcatgggcccaacgtgagtggaccattcagcatgtcttgtttccatccatgcgacttttcctcaagcccccgaagtcaatggcaacagatggaacgtttgtgcaggttgcttccttggagaaactctacaagatttttgaaaggtgttagctcataagtgagaaaatgaaggcagagtaagatcatgattcatggagtgtttttgaaaatgtgtataatttcaccgtattatgtactttgatagtgtctgtagaaactgaagaaagaaagatggctttacttctgaattgaaagttaacgatgccagcaattgtatattctgatcaaccaaaaaaaaaaaaaaaaaaaaaaaaaaa

SEQ ID NO: 59 - protein, 724 aa, Oryza sativa
MDEPSPRGGGCAGEPPRIRRLEESVVNRIAAGEVIQRPSSAVKELIENSLDAGASSVSVAVKDGGLKLIQVSDDGHGIRFEDLAILCERHTTSKLSAYEDLQTIKSMGFRGEALASMTYVGHVTVTTITEGQLHGYRVSYRDGVMENEPKPCAAVKGTQVMVENLFYNMVARKKTLQNSNDDYPKIVDFISRFAVHHINVTFSCRKHGANRADVHSASTSSRLDAIRSVYGASVVRDLIEIKVSYEDAADSIFKMDGYISNANYVAKKITMILFINDRLVDCTALKRAIEFVYSATLPQASKPFIYMSIHLPSEHVDVNIHPTKKEVSLLNQERIIETIRNAIEEKLMNSNTTRIFQTQALNLSGIAQANPQKDKVSEASMGSGTKSQKIPVSQMVRTDPRNPSGRLHTYWHGQSSNLEKKFDLVSVRNVVRSRRNQKDAGDLSSRHELLVEIDSSFHPGLLDIVKNCTYVGLADEAFALIQHNTRLYLVNVVNISKELMYQQALCRFGNFNAIQLSEPAPLQELLVMALKDDELMSDEKDDEKLEIAEVNTEILKENAEMINEYFSIHIDQDGKLTRLPVVLDQYTPDMDRLPEFVLALGNDVTWDDEKECFRTVASAVGNFYALHPPILPNPSGNGIHLYKKNRDSMADEHAENDLISDENDVDQELLAEAEAAWAQREWTIQHVLFPSMRLFLKPPKSMATDGTFVQVASLEKLYKIFERC

SEQ ID NO: 60 - DNA, 287 bp, Conyza sp.
cacatctttagcatcggccaccattgaaaaagtggctgaatcatgggataaaaatgtcgctacaagtgttgatgatggtagggacttgaatgattctaatggtgatggccttcactcgactgttgaaccaacattgcgtggtttgcatgcatatgttggtgattctaatgtacctccaaaccaaagttccctcctgatgcttcttattttcaaccggctgcatgtcatgcaaatgacattcaccctgctacagatgaggcccctttgcatgatgtttctccgaatga

SEQ ID NO: 61 - DNA, 348 bp, Conyza sp.
atggtagggatttgaatgattcgactggggatggcttacactcgactgctgaaccaacattgcatggtttgcatgcaaatgttgatgattgtactgtgcctcctatgccggaaccgccaaagttccctcctgatgctacttactttcagccggctgcatgtcatgtaaatgacattcatcctgcttcacatgaggccccttatgcatgatgttactcctaatgatcttagtggataccctgacagtcctaaggtccagcagccgcgtacttatgcttctatctttcaggatgcggctaacatcaacaagaaaggtaaattgagattcatccctccaaaaaaaaaaaaa

SEQ ID NO: 62 - DNA, 18 bp, Artificial Sequence
  Oligonucleotide primer
cacatctttagcatcggc

SEQ ID NO: 63 - DNA, 20 bp, Artificial Sequence
  Oligonucleotide primer
tcattcggagaaacatcatg

SEQ ID NO: 64 - DNA, 52 bp, Artificial Sequence
  Oligonucleotide primer
aagcagtggtatcaacgcagagtactttttttttttttttttttttttttvn

SEQ ID NO: 65 - DNA, 26 bp, Artificial Sequence
  Oligonucleotide primer
cacatctttagcatcggccaccattg

SEQ ID NO: 66 - DNA, 45 bp, Artificial Sequence
  Oligonucleotide primer
ctaatacgactcactatagggcaagcagtggtatcaacgcagagt

SEQ ID NO: 67 - DNA, 22 bp, Artificial Sequence
  Oligonucleotide primer
ctaatacgactcactatagggc

SEQ ID NO: 68 - DNA, 25 bp, Artificial Sequence
  Oligonucleotide primer
gtggctgaatcgtggtataagaatg

SEQ ID NO: 69 - DNA, 23 bp, Artificial Sequence
  Oligonucleotide primer
aagcagtggtatcaacgcagagt

SEQ ID NO: 70 - DNA, 48 bp, Artificial Sequence
  Oligonucleotide primer
gtaatacgactcactatagggcacgcgtggtcgacggcccgggctggt

SEQ ID NO: 71 - DNA, 12 bp, Artificial Sequence
  Oligonucleotide primer
aattaccagccc

SEQ ID NO: 72 - DNA, 12 bp, Artificial Sequence
  Oligonucleotide primer
gatcaccagccc

SEQ ID NO: 73 - DNA, 12 bp, Artificial Sequence
  Oligonucleotide primer
agctaccagccc

SEQ ID NO: 74 - DNA, 22 bp, Artificial Sequence
  Oligonucleotide primer
gtaatacgactcactatagggc

SEQ ID NO: 75 - DNA, 22 bp, Artificial Sequence
  Oligonucleotide primer
gagagagagagagagagagagb

SEQ ID NO: 76 - DNA, 21 bp, Artificial Sequence
  Oligonucleotide primer
gagagagagagagagagagah

SEQ ID NO: 77 - DNA, 19 bp, Artificial Sequence
  Oligonucleotide primer
actatagggcacgcgtggt

SEQ ID NO: 78 - DNA, 546 bp, Conyza sp.
tagggcgaattgggccctctagatgcatgctcgagcggccgccagtgtgatggatatctgcagaattcggcttactagagggcacgcgtggtcgacggcccgggctggtgatctcataccagctgaccatcgtatcatcatgtgccaattagcttgcaaaagttctgaatttgtaatggttgatagctgggaggtctctctctctctctctctctctctgacacacacacacacacacaaatatgttttactaaagctctactttaacatattgcaatttacttttatgactaaagcatgttgaatgtagaataagttctttttttagggcagatgtagatctgatttactgataattaatatcgacctctttgctgtacaagacctctgcttatttttctttctatataatgatgcagagccatttatcttttttaggcaaaccaaagttcgtttcaacgctcattgacagtattgtcaagaataaggagtttcttttgtgataacggactaatatctagtggtatgttctaatgttctatatcc

SEQ ID NO: 79 - DNA, 445 bp, Conyza sp.
actagagggcacgcgtggtcgccccgggctggtgatccatctgttgttagaggagagaaacagaagaatggacttgctcttcctcttgctggaatgaattcgacgttggttcatggtgatgactcggcagcggagtttggtgcgagagagagagacagtaagaagatgtgtgtgtatggagaataaaagggataatgtggaagggataataaggaacggaaaagggtataccaaaaaggaaaatgtccaagggcaatattggtattttaaaggcagaagttactaaaaatagaaaagggtgttttgctttattaggtagtaaagattgatatcttaaagtgctaatttgactattatttagtttgtcatataaaactttttcctatattgcaacttcatgtttctcattactaaagatttgacaagtgttatgcaatttaggatc

SEQ ID NO: 80 - DNA, 454 bp, Conyza sp.
gatcctccatgggtcaaacccatgacccatcacccaactaacttacccaaccctatcttccttattttccacccatccattcattcttcaccctatctttttctttccttcttcttccaagaacaaaacacacatacacactattgtctctctctctctctctctctctctctaagattcttacaccaaattatggtttcaagttagaattgtgttagaatcatcatccttatcatcatcttcattacatatcaaagatttgggttgtcaaaagtgcaagaacatcttcttttgagtaattttcgggtttggcaatttgtggaagttttggtaagtgaattaccagcccggggccgtcgaccacgcgtgccctttagttccattcattcttcaccctatctttttctttctttcctcttgcaagaacaaaacacacatacacactaatgtctct

SEQ ID NO: 81 - DNA, 254 bp, Conyza sp.
agcttaggtgtggaatctcacatactaattttttaatattgtttgaattttaaataaatcacatggcccccatgatatttaaagattaaaaaaagaatatgttagtgttctacacctaagcttagatacgtaacaaccttatattcccatccctctctctctctctctctgttggatattgtaaacaactattcaaataaaacaaatagggcaaggtcaccagcccgggccgtcgaccacgcgtgccctgtagt

SEQ ID NO: 82 - DNA, 247 bp, Conyza sp.
actagagggcacgcgtggtcgacggcccgggctggtgatctcttaaaaaatatagacatccattccataaatattcataacaacaaacaaaatgataattatttagatatagatatagggtataaaatgagagagagagagagagaaagcgtgtgtgcatacgcgtattgaagagtagcaagtcaattgaggtcaggtttagggtttggaagagtcgtttttcattgatataaccgaaaaacggatc

SEQ ID NO: 83 - DNA, 339 bp, Conyza sp.
agctttgaagacttgtttcatcacaccaattgtcttcaccatcgggctcatcaacttaattcctacaagacaaaataacacgtacttcattcattacaatatatcactaagggggagaatgttagaaattcaaaaatagtgataaattgtaatttaagttagtggatgttattgttgttaagacatggtgaaatgatttctaaggttgagggactaggagtgtcaattagcttaattgtagatttagactataaataccctcaactcattagaaatcataagcctttaatccatttttacatacaacttattcagatcgtctctctctctctctctctc

SEQ ID NO: 84 - DNA, 373 bp, Conyza sp.
gatccttattttgagaacacctttttctgtaagaaacttgagaactcttaagttatgtattggatcacattttttttgttcatcctcatgtgaaacgttataaaaatatttgtagaaataagtttttttcaaaaccttatatatacaagtttgatcacatgtgaacgataacgtgaacatattgattcacaagttcacaaaagctattttgatgggttttttaaaaagtttattcttcaacatacttttttatatcatttcacattaaattaacaaaaaaacatgtgatccaatacataatttgagagttctcgtggttcttacaaaaaaaatggttttcaaaataaggaaagtctctctctctctctctctc

SEQ ID NO: 85 - DNA, 398 bp, Conyza sp.
aattcacatcatcaatggcggattggaagtgtgtgtgttcttggtgtgagtgtgtgttttgagagagaaaatgtgtgtgaaaggaaagtgctttattggaattaatgagaattgtgaaggttgcaatggttttaaaaaggctcaaggcttatgatttctaaggtgtgaggggtatttataggctaactacacaaatatgctaattatcacatttagcccttcaaccttagtgatcatttacctatgacttaactacaagtaagtccactaatctaaattacaatttatcactattttggatttctaacaattgttttatacaataaaaagagatgacgaccttttaccgacaaatttttttttaggactctctctcactctctctctctctctctctc

SEQ ID NO: 86 - DNA, 303 bp, Conyza sp.
gagagagagagagagagagacacacacacagagtgggtggggagtaagactgagagacgggaaggaattatcggatcggaaggaaggaaaaatgacaagcgcattagtattcatggctcttgaatgcttcaaatgctccaccatgcaagtaagtcctcctcttcgatcgtaattgatggatgtgatttgattaaaaatgatggatggacaacaggtgaagcagaggaagaaaagcagcaacaaatggatctgcgttatctgcaacgagaagcagtccgtccggaaggtctattcccagggatc

SEQ ID NO: 87 - DNA, 397 bp, Conyza sp.
  misc_feature (336)..(336): Unknown nucleotide
gagagagagagagaggagagagacctcccttgatagaattacctatttcaaatacccaattgacggaaactcccaactaatccgttcgaaagcacgacgattaatagggctaaaccctgccggctcagacttgaacgtgtttggtcttttatttatagttcttgtattaactggtcacatgaatctataatagattctataaagataacgaaaaaagagtttctctaaattgttgcactggaattgacgaagacttaataccaatattattctttatttccaagccctcatagaattaatatatatatatatctcaaggttggttgaataaggatntaaactttaaacctataagtcatctggataatttcaaaccattacactaactactcatcat

SEQ ID NO: 88 - DNA, 246 bp, Conyza sp.
  misc_feature (181)..(181): Unknown nucleotide
gatccgattgaggacggcggaagcgaatacgacatagcagaagaagatgaaaaaaccactaatcatctccgtgatcgttttcgcctttccacaatctctattgctcaatctgaaggtctctctctctctctctctctctctctctatctctctctatctctctctctctctctctctctanatctctctctctctctctctctctctctctttctctctctctctgtctctctctctctctctctc

SEQ ID NO: 89 - DNA, 184 bp, Conyza sp.
gagagagagagagagagagacttacgaagaagatgaaggttttgaagaaaatgaaaaaatatttaccttttggaagaacaatatagatctagatcaggtaggtgaaggttttgaagatgatggaattatgttgcagctagggttcatttgtggtgggaggggcttttggtctattgtctgaatt

SEQ ID NO: 90 - DNA, 221 bp, Conyza sp.
gatccattcccgggactcaattaggtcaaggccaaatgtaaaacacatatatatctcgacatatactccgggagtgagttaaggaaacatcctctttatctttcactctagtttctctttcctcgagtgagctctcggctggaccccaccctctagctctcgatctactcctttctttaagtttttggtttacttttctcatctctctctctctctctctc

SEQ ID NO: 91 - DNA, 104 bp, Conyza sp.
gagagagagagagactgagagtgttgcggtcaatggatcgaatcctgatccggcggatgcaaaggatactccggtcatgaggtcttgaatcgtaagtgttggat

SEQ ID NO: 92 - DNA, 286 bp, Conyza sp.
gatccaacttgagatgatgtatccatatgcggtttttttggacttgatcgaattgagagaattagagatctgaaagttaaaatttgggaagtcattccaagaggaccccatctttagagagatcaatgtgatgcttacatatgacaacaataatcagggctgggcagtggtatggcatggcctgatagaagatgactcaaagagcttgactaattgggtgagggtggatgactctcttaaagagcttcactattctttctactccttctctctctctctctctctc

SEQ ID NO: 93 - DNA, 153 bp, Conyza sp.
gatcctccatgggtcaaacccatgacccatcacccaaccctatcttccttattttccacccatccattcattcttcaccctatctttttctttctttcttcttgcaagaacaaaacacacatacacactaatgtctctctctctctctctctc

SEQ ID NO: 94 - DNA, 153 bp, Conyza sp.
gagagagagagagagagagacattagtgtgtatgtgtgttttgttcttgcaagaagaaagaaagaaaaagatagggtgaagaatgaatggatgggtggaaaataaggaagatagggttgggtgatgggtcatgggtttgacccatggaggatc

SEQ ID NO: 95 - DNA, 509 bp, Conyza sp.
  misc_feature (481)..(481): Unknown nucleotide
gagagagagagagagagagacgatctgatttgttgtatgtgtaaaaatggataaaaggcttattggttgttgctatgatttctaatgtattgagggtatttatactctaatattacaaatatccttattgtcatatctaacccctcaacattagaaatcatttcaccatgtctaaacaacaattaagtccactaacttaaattacaatttatcactatttttggatttctaacaatcactgtttgtcactccatcgaatttctcttcctcaacactcttcaaatgatttccttttaagagtaaaattcaacccggtggctggagaacgaatagcattgccacggttggccggtatatttctccttcgatccccgtaaagaaggttgttggggttgactggttgttcctgatcaccaattcttcttgcctgaactggttgatttacccgaagaattactgttgaaccaggtatagaagtctngtccttattatcgtccttttctgaacta

SEQ ID NO: 96 - DNA, 343 bp, Conyza sp.
  misc_feature (207)..(207): Unknown nucleotide
gagagagagagagagagagacgatacctgagattttgggcggcaagaagtgaggcgaaattcctttgaagattgtttgatggtgaaagtgaagtgggatgcaggttgaaaacaaaggagtaaaaccctattatttaccgatgaagaggttaacagctgaatggttttagacctaacttgttaacggttgaagttaagagacggtacngataacttaaaagaataccaaaattataaatttaacttttttaatatttaagttaaaataaatttaattttttcgataccttttttccccatcatccctataattcaaaatcataaaaggatgtgtgtaatggatc

SEQ ID NO: 97 - DNA, 111 bp, Conyza sp.
gagagagagagagagagagactgagagtgttgcggtcaatggatcgaatcctgatccggcggatgcaaaggatactccggtcatgaggtcttgaatcgtaagtgttggatc

SEQ ID NO: 98 - DNA, 60 bp, Conyza sp.
cgacggccccgggctggtgatccacatcaacggtcacacgtctctctctctctctctctc

SEQ ID NO: 99 - DNA, 158 bp, Conyza sp.
atattagtctaagtgctggaatctaaatttgcctcccacaccccctttatctcagattttatttcctccaaccctccatttcttatctacctttctctctctaattctcttccatgaacacacacacaaacatacctgtctctctctctctctctctc

SEQ ID NO: 100 - DNA, 558 bp, Conyza sp.
gatccctatccgtttcaaatatagtaacaaaatgttaactatttcacagagtatatattttcctagcaacgtgatttagtacactttgggtgattttctacatatacctacctttttggctatttttctaatttacctgtgtgctgttttagatacagaacacatagcatgcatttgagtatcgcctcatttataagtaactcggtcaaagttgtcatctctcgagagttcccataagttaattctctttttggttcttttaggtgaacaaaggaactgctagtgcaagtgagattttagctggtgcattgaaggataacaagcgtgcagtgcttcttggagaacccacttacggcaaagggtaatcaattttgcataatgcttcttaatagcataacatccttgatcagatgtctcaggaataaatgatccttattcacagaaaaatccaatcggtttttgaattgtctgatggctctggcttggctgttactgttgctcgatatgaaacccctgatcacattgacatcaataaggttctctctctctctctctctc

SEQ ID NO: 101 - DNA, 166 bp, Conyza sp.
gatcctccatgggtcaaacccatgacccatcacccaactaacttacccaaccctatcttccttattttcaacccatccattcattcttcaccctatctttttctttccttcttcttccaagaacaaaacacacatacacactattgtctctctctctctctctctc

SEQ ID NO: 102 - DNA, 390 bp, Conyza sp.
gagagagagagagagaaggagagagagagagagagagagaaggagagagagagagagagagagagagagaagagagagagagagagagagatagagagagagtgagagagagagagagacgatctaaatgtaaaaatggatgaaaggcttattgaggtgttgttatgatttctaatgtatagagggtatatatactctaatattacaaatatccttattgtcatatctaacccctcaacgttagaaatcatttcaccatgtctaaacaacaattaagtccactaacttaaattacaagttatcactatttttggatttctaacagaaatgttgtagacttttatttggtttagttgcaactatttgatggaatgtaaatggagagggatc

SEQ ID NO: 103 - DNA, 413 bp, Conyza sp.
tctctctctctctctctctcaagccgaattccagcacactggcggccgttactagtggatccgagctcggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatc

SEQ ID NO: 104 - DNA, 405 bp, Conyza sp.
gagagagagagagagggagagagagagagcagagagacaaagagacatacagcaaaccatataaactccttggttgcaagaggtcttgtaatttcatgccttttcagtgattcttcaacagtgccttgaaccttcacatagaacaaaaccttttaatattgatttagattaaaatgagctaagatagtatttgtcccactgaaagaaaagcataagttacatttaagagaaccaaaatttatgtattagctacaaagctaacagttgatcaatacattctataagtgagtcggttctgggtgtttggtggttaattttaggtaccgtgaatccgtgatccactgctgcaacacttggagcaacatcgtttggaaaaacatatgaggactatttgcttgtttctcc

SEQ ID NO: 105 - DNA, 385 bp, Conyza sp.
gagagagagagagagagagacattagtgtgtatgtgtgtattgttcttgcaagaagaaagaaagaaaaagatagggtgaagaatgaatggatgggtggaaaataaggaagatagggttgggtaagtaagttgggtgatgggtttgacccatggaggatcaccagcccggggccgtcgaccacgcgtgccctttagtaagccgaatgccagcacactggcggccgttactagtggatccgagctcggtaccaagcttggcgtaatcatggtcatagctgtttcctgtgtgaaattgttatccgctcacaattccacacaacatacgagccggaagcataaagtgtaaagcctggggtgcctaatgagtgagctaactcacatgaat

SEQ ID NO: 106 - DNA, 347 bp, Conyza sp.
gagagagagagagagagagagagagagacgatctgaataagttgtatgtaaaagtggataaaaggcttatgatttctaaggagttgagggtatttatagtctaaatctacaattaagctaattgacactcctagtccctcaaccttagaaatcatttcaccatgtcttaacaacaacaacgtccactaacttaaatttcaatttatcactatttttgaatttctaaaattctctcccttagtgatatattgtaatgaatgaagtacgtgttattttgtcttgtaggaattaagttgatgagcccgatggtgaagacaattggtgtgatgaaacaagtcttcaaagct

SEQ ID NO: 107 - DNA, 152 bp, Conyza sp.
gagagagagagagagagagacctggaagaagccccattctttagaagctgcgtgtagcttattcagctcatcagaagaaatgtcactggaagtgtttgcaaagaggttttgaaggtcgatgaccggcacggatgatgtggtagtagtcaacg

SEQ ID NO: 108 - DNA, 88 bp, Conyza sp.
gagagagagagagagagagacgatctgaataagttgtatgtaaaaatggatcaccagcccgggccgtcgaccacgcgtgccctttagt

SEQ ID NO: 109 - DNA, 314 bp, Conyza sp.
actaaagggcacgcgtggtcgacggcccgggctggtgatcaaatgaaagttgacatactttgtgaaattgtgtataaattattcataagcaaacaaggaagctctttattttcatcgatctctttttgggtacgtctaaccttttttttgttatgatctaaggccggttcttataatagaaggagtccttaggagtcctgactgccacgtcaacaataggactctccttaggactctccctgcctataatgacaagcttttttagatcttggtcccacctctttactttctccctctctctctctctctctctc

SEQ ID NO: 110 - DNA, 438 bp, Conyza sp.
gagagagagagagagagagagcgggggagttagggtaaaataaaaaactattaacctaaaataagtaagacatcacaaaaaagtgacatgtggcatgagaatatttaaaattaaattataatttcaagggtaatatggacattatgtaaaattaatatctggcttctataattgatgtaacccatcgtgtattagaccctaatttgttagaaatccaaaaatagtgataaattgtaatttaagttagtggatgttattgttgttaagacatggtgaaatgatttctaatgttgaggggttagatatgacaataaggatatttgtaatattagagtataaataccctcaatacattagaaatcataacaacaattaagccttttatccattttacaaatacaacatatatagatcgcctctctctctctctctctctct

SEQ ID NO: 111 - DNA, 202 bp, Conyza sp.
gagagagagagagagagagagggggagttagggtaaaataaaaaactattaacctaaaataagtaagacatcataaaaaagtgacatgtggcatgagaatatttaaaattaaattataatgtcaagggtaatatggacattatgtaaaattaatatctggcttctataattgatgtaacccatcgtgtataagaccctaatt

SEQ ID NO: 112 - DNA, 91 bp, Conyza sp.
agagagaggagagagagagggccgatctgaataagttgtatgtaaaaaatggatcaccagcccgggccgtcgaccacgcgtgccctctagt

SEQ ID NO: 113 - DNA, 19 bp, Artificial Sequence
  Oligonucleotide primer
ccatcgtatcatcatgtgc

SEQ ID NO: 114 - DNA, 18 bp, Artificial Sequence
  Oligonucleotide primer
tagcttgcaaaagttctg

SEQ ID NO: 115 - DNA, 21 bp, Artificial Sequence
  Oligonucleotide primer
tgcaatatgttaaagtagagc

SEQ ID NO: 116 - DNA, 18 bp, Artificial Sequence
  Oligonucleotide primer
taccaatattgcccttgg

SEQ ID NO: 117 - DNA, 19 bp, Artificial Sequence
  Oligonucleotide primer
gtatacccttttccgttcc

SEQ ID NO: 118 - DNA, 22 bp, Artificial Sequence
  Oligonucleotide primer
ttcatggtgatgactcggcagc

SEQ ID NO: 119 - DNA, 18 bp, Artificial Sequence
  Oligonucleotide primer
tacccaaccctatcttcc

SEQ ID NO: 120 - DNA, 18 bp, Artificial Sequence
  Oligonucleotide primer
tccattcattcttcaccc

SEQ ID NO: 121 - DNA, 21 bp, Artificial Sequence
  Oligonucleotide primer
ccataatttggtgtaagaatc

SEQ ID NO: 122 - DNA, 18 bp, Artificial Sequence
  Oligonucleotide primer
atgttagtgttctacacc

SEQ ID NO: 123 - DNA, 18 bp, Artificial Sequence
  Oligonucleotide primer
cttagatacgtaacaacc

SEQ ID NO: 124 - DNA, 18 bp, Artificial Sequence
  Oligonucleotide primer
aacgactcttccaaaccc

SEQ ID NO: 125 - DNA, 18 bp, Artificial Sequence
  Oligonucleotide primer
tgacctcaattgacttgc

SEQ ID NO: 126 - DNA, 18 bp, Artificial Sequence
  Oligonucleotide primer
atatagacatccattcca

SEQ ID NO: 127 - DNA, 259 bp, Conyza sp.
cacatctttagcatcggccaccattgaaaaagtggctgaatcgtggtataagaatgttgtattgcaggttgatgttgagagggatttggatgatttgaatggtggtgccagaattctactgctgagtcatctttgcatgatttccatgcaaaaggtggtgctactcatgtttcccctatgcttgatcctcctaagtttcctcctggtactacttattttaagccagctacagacacatgccaatgacattcttgatgtt

SEQ ID NO: 128 - DNA, 287 bp, Conyza sp.; identical to SEQ ID NO: 60

SEQ ID NO: 129 - DNA, 348 bp, Conyza sp.; identical to SEQ ID NO: 61